(19) United States
(12) Patent Application Publication
(10) Pub. No.: US 2011/0003776 A1
(43) Pub. Date: Jan. 6, 2011

(54) TETRAHYDROPYRANONAPHTHYRIDINES DERIVATIVES, PHARMACEUTICAL COMPOSITIONS AND THERAPEUTIC TREATMENT THEREOF

(75) Inventors: John K. Snyder, Harvard, MA (US); John A. Porco, JR., Brookline, MA (US); Ya Zhou, Nashville, TN (US)

Correspondence Address: RONALD I. EISENSTEIN, 100 SUMMER STREET, NIXON PEABODY LLP, BOSTON, MA, 02110, US

(73) Assignee: TRUSTEES OF BOSTON UNIVERSITY, Boston, MA (US)

(21) Appl. No.: 12/793,422

(22) Filed: Jun. 3, 2010

Related U.S. Application Data
(60) Provisional application No. 61/183,838, filed on Jun. 3, 2009.

(51) Int. Cl. A61K 31/65; C07D 491/14; A61K 31/4375; A61K 31/5377; A61P 31/06; A61P 31/04; A61K 31/397; A61K 31/496

(52) U.S. Cl. 514/154; 546/83; 514/293; 544/105; 514/230.5; 514/210.05; 514/254.11

(57) ABSTRACT

This invention relates to tetrahydropyranonaphthyridines derivatives having formula (III) or IV:

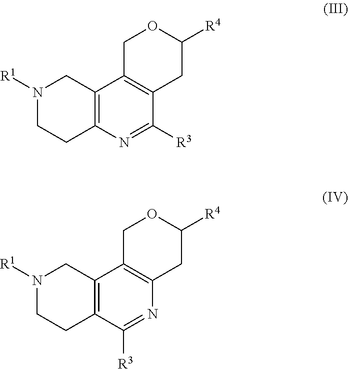

and analogues of the tetrahydropyranonaphthyridines derivatives. The invention also relates to pharmaceutical compositions comprising these compounds and methods for treatment of tuberculosis using these compounds.

### CROSS-REFERENCE TO RELATED APPLICATIONS

This application claims benefit under 35 U.S.C. §119(e) of the U.S. Provisional Application No. 61/183,838, filed Jun. 3, 2009, the content of which is herein incorporated by reference in its entirety.

### GOVERNMENT SUPPORT

This invention was made with government support under Contract No. GM067041 awarded by the National Institutes of Health. The U.S. government has certain rights in the invention.

### FIELD OF THE INVENTION

This invention relates to tetrahydropyranonaphthyridines derivatives and analogues thereof, pharmaceutical compositions comprising these compounds and methods for treatment of tuberculosis using these compounds.

### BACKGROUND OF THE INVENTION

Tuberculosis (TB) is a common and deadly infectious disease caused by mycobacteria, mainly Mycobacterium tuberculosis. Over one-third of the world's population has been exposed to the TB bacterium, and new infections occur at a rate of one per second. In 2006, TB was reported to kill up to 5,000 people worldwide each day, with 1.5 million people estimated to have succumbed to TB that year. Today, TB remains the leading cause of death worldwide due to infectious disease. In 2004, mortality and morbidity statistics included 14.6 million chronic active TB cases, 8.9 million new cases, and 1.6 million deaths, mostly in developing countries. In 2005, the infection rate in the United States was 4.8 cases per 100,000, and was estimated to be significantly higher among foreign-born residents. Tuberculosis most commonly attacks the lungs (as pulmonary TB), but can also affect the central nervous system, the lymphatic system, the circulatory system, the genitourinary system, bones, joints and even the skin. In addition, a rising number of people in the developed world are contracting tuberculosis because their immune systems are compromised by immunosuppressive drugs, substance abuse, or HIV/AIDS. The rise in HIV infections and the neglect of TB control programs have enabled a resurgence of tuberculosis. The emergence of drug-resistant strains has also contributed to this new epidemic with, from 2000 to 2004, 20% of TB cases being resistant to standard treatments. A dire need exists for new treatments for tuberculosis, particularly in light of the emergence of drug resistant strains of M. tuberculosis.

An additional; critical problem is the ability of the organism to lie in a currently untreatable dormant phase, only to emerge months after treatment has ceased. As a consequence, primary treatment of tuberculosis requires a daily dosage of a four drug combination therapy for up to two months, followed by a continued phase 2 treatment up to at least four additional months.

These two issues, drug resistance and dormancy, are extremely difficult to address medically with today's drug regimen. These problems highlight a general challenge in drug discovery—new chemotypes, i.e., a need for new compounds with unique structures that have not been previously conceived through synthesis or found in nature. New types of compounds with distinct structures to combat M. tuberculosis thus represent an ongoing need. The compounds described in this invention fill this need.

### SUMMARY OF THE INVENTION

This invention relates to a compound having a formula (III) or (IV):

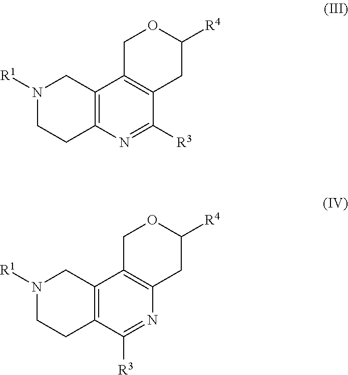

wherein:

R¹ is —(CH₂)ₘR⁶, —C(O)N(H)R⁶, —C(O)R⁶, or —S(O)₂R⁶;

m is 0-6;

R³ and R⁴ are each independently a substituted or unsubstituted alkyl, substituted or unsubstituted aryl, or substituted or unsubstituted heterocylic ring containing one or more heteroatoms; and

R⁶ represents independently for each occurrence a substituted or unsubstituted alkyl, substituted or unsubstituted cycloalkyl, substituted or unsubstituted alkenyl, substituted or unsubstituted cycloalkenyl, substituted or unsubstituted aryl, substituted or unsubstituted alkoxy, substituted or unsubstituted aryloxy, substituted or unsubstituted heterocylic ring containing one or more heteroatoms, or substituted or unsubstituted fused ring formed between two or more cyclic rings or heteroatom-containing cyclic rings.

The invention also relates to a pharmaceutical composition, comprising a therapeutically effective amount of a compound of formula (III) or (IV), in association with a pharmaceutically acceptable carrier or excipient.

The invention also provides a method for treating tuberculosis, bacteria infection caused by tuberculosis, or related diseases, which comprises administering a therapeutically effective amount of a compound of formula (I) or (II), or a salt thereof to a subject in need of such treatment:

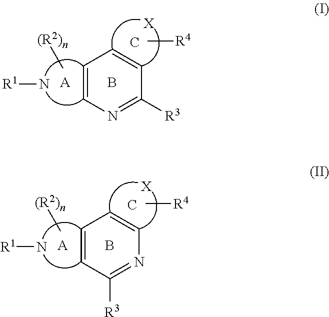

wherein

A and C are each independently a 5-7 member heterocyclic ring, wherein the N and X variables are present at any position in the ring;

R¹ is H, substituted or unsubstituted alkyl, substituted or unsubstituted aryl, —(CH₂)ₘR⁶, —C(O)N(H)R⁶, —C(O)R⁶, or —S(O)₂R⁶;

m is 0-6;

each R² is independently H, alkyl, or aryl;

n is 0-4;

R³ is hydrogen, substituted or unsubstituted alkyl, substituted or unsubstituted aryl, or substituted or unsubstituted heterocylic ring containing one or more heteroatoms;

R⁴ is hydrogen, substituted or unsubstituted alkyl, substituted or unsubstituted aryl, or substituted or unsubstituted heterocylic ring containing one or more heteroatoms, wherein R⁴ is in an ortho-position to X on the heterocyclic ring and with the proviso that R⁴ is H when C is a 5-member ring;

X is O, S, S(O), S(O)₂, or NR⁵, wherein R⁵ is substituted or unsubstituted alkyl or aryl; and

R⁶ represents independently for each occurrence a substituted or unsubstituted alkyl, substituted or unsubstituted cycloalkyl, substituted or unsubstituted alkenyl, substituted or unsubstituted cycloalkenyl, substituted or unsubstituted aryl, substituted or unsubstituted alkoxy, substituted or unsubstituted aryloxy, substituted or unsubstituted heterocylic ring containing one or more heteroatoms, or substituted or unsubstituted fused ring formed between two or more cyclic rings or heteroatom-containing cyclic rings.

### DETAILED DESCRIPTION OF THE INVENTION

This invention relates to a new chemical structure type with demonstrated activity against the tuberculosis-inducing Mycobacterium tuberculosis. The compounds of the invention represent a distinct structural difference from traditional drugs, and have the potential to serve as new generation of anti-tuberculosis drug.

### TERMINOLOGY AND DEFINITIONS

Unless otherwise indicated, the disclosure is not limited to specific reactants, substituents, catalysts, reaction conditions, or the like, as such may vary. It is also to be understood that the terminology used herein is for the purpose of describing particular embodiments only and is not intended to be limiting. Therefore, unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments of the aspects described herein, and are not intended to limit the claimed invention, because the scope of the invention is limited only by the claims. Further, unless otherwise required by context, singular terms shall include pluralities and plural terms shall include the singular.

As used in the specification and the appended claims, the singular forms “a,” “an,” and “the” include plural referents unless the context clearly dictates otherwise. Thus, for example, reference to “a substituent” encompasses a single substituent as well as two or more substituents, and the like.

As used in the specification and the appended claims, the terms “for example,” “for instance,” “such as,” or “including” are meant to introduce examples that further clarify more general subject matter. Unless otherwise specified, these examples are provided only as an aid for understanding the applications illustrated in the present disclosure, and are not meant to be limiting in any fashion.

The term “alkyl” as used herein refers to a linear, branched, or cyclic saturated hydrocarbon group typically although not necessarily containing 1 to about 24 carbon atoms, preferably 1 to about 12 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although again not necessarily, alkyl groups herein contain 1 to about 12 carbon atoms. The term “cycloalkyl” intends a cyclic alkyl group, typically having 4 to 10, preferably 5 to 7, carbon atoms. The term “substituted alkyl” refers to alkyl substituted with one or more substituent groups, and the terms “heteroatom-containing alkyl” and “heteroalkyl” refer to alkyl in which at least one carbon atom is replaced with a heteroatom. If not otherwise indicated, the term “alkyl” includes linear, branched, cyclic, unsubstituted, substituted, and/or heteroatom-containing alkyl, respectively.

The term “alkylene” as used herein refers to a difunctional linear, branched, or cyclic alkyl group, where “alkyl” is as defined above.

The term “alkenyl” as used herein refers to a linear, branched, or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Preferred alkenyl groups herein contain 2 to about 12 carbon atoms. The term “cycloalkenyl” intends a cyclic alkenyl group, typically having 4 to 10, preferably having 5 to 8 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the term “alkenyl” includes linear, branched, cyclic, unsubstituted, substituted, and/or heteroatom-containing alkenyl, respectively.

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 24 carbon atoms, and particularly preferred aryl groups contain 5 to 14 carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituents in which at least one carbon atom is replaced with a heteroatom, as will be described in further detail infra.

The term “alkoxy” as used herein intends an alkyl group bound through a single, terminal ether linkage; that is, an “alkoxy” group may be represented as —O-alkyl where alkyl is as defined above.

The term “aryloxy” as used herein refers to an aryl group bound through a single, terminal ether linkage, wherein “aryl” is as defined above. An “aryloxy” group may be represented as —O-aryl where aryl is as defined above. Preferred aryloxy groups contain 5 to 24 carbon atoms, and particularly preferred aryloxy groups contain 5 to 14 carbon atoms. Examples of aryloxy groups include, without limitation, phenoxy, o-halo-phenoxy, m-halo-phenoxy, p-halo-phenoxy, o-methoxy-phenoxy, m-methoxy-phenoxy, p-methoxy-phenoxy, 2,4-dimethoxy-phenoxy, 3,4,5-trimethoxy-phenoxy, and the like.

The term “alkylaryl” refers to an aryl group with an alkyl substituent, and the term “arylalkyl” refers to an alkyl group with an aryl substituent, wherein “aryl” and “alkyl” are as defined above. Preferred alkylaryl and arylalkyl groups contain 6 to 24 carbon atoms, and particularly preferred alkylaryl and arylalkyl groups contain 6 to 16 carbon atoms. Alkylaryl groups include, for example, o-methylphenyl, p-methylphenyl, m-methylphenyl, 2,4-dimethylphenyl, 2,4-dimethylphenyl, 3,4-dimethylphenyl, 3,5-dimethylphenyl, 2,4,6-trimethylphenyl, p-cyclohexylphenyl, 2,7-dimethylnaphthyl, 7-cyclooctylnaphthyl, 3-ethyl-cyclopenta-1,4-diene, and the like. Examples of arylalkyl groups include, without limitation, benzyl, phenyl-methyl, 1-phenyl-ethyl, 2-phenyl-ethyl, 1-phenyl-propyl, 2-phenyl-propyl, 3-phenyl-propyl, 1-phenyl-butyl, 2-phenyl-butyl, 3-phenyl-butyl, 4-phenyl-butyl, 4-phenylcyclohexyl, 4-benzylcyclohexyl, 4-phenylcyclohexylmethyl, 4-benzylcyclohexylmethyl, and the like.

The term “acyl” refers to substituents having the formula —(CO)-alkyl, —(CO)-aryl, or —(CO)-aralkyl, and the term “acyloxy” refers to substituents having the formula —O(CO)-alkyl, —O(CO)-aryl, or —O(CO)-aralkyl, wherein “alkyl,” “aryl, and “aralkyl” are as defined above.

The terms “halo” and “halogen” are used in the conventional sense to refer to a chloro, bromo, fluoro or iodo substituent.

The term “heteroatom-containing” as in a “heteroatom-containing hydrocarbyl group” refers to a hydrocarbon molecule or a hydrocarbyl molecular fragment in which one or more carbon atoms is replaced with an atom other than carbon, e.g., nitrogen, oxygen, sulfur, phosphorus or silicon; typically nitrogen, oxygen or sulfur. Similarly, the term “heteroalkyl” or “heteroalkenyl” refers to an alkyl or alkenyl substituent that is heteroatom-containing, respectively; the term “heterocyclic” refers to a cyclic substituent that is heteroatom-containing; the terms “heteroaryl” and “heteroaromatic” respectively refer to “aryl” and “aromatic” substituents that are heteroatom-containing, and the like. It should be noted that a “heterocyclic” group or compound may or may not be aromatic, and further that “heterocycles” may be monocyclic, bicyclic, or polycyclic as described above with respect to the term “aryl.” Examples of heteroalkyl groups include alkoxyaryl, alkylsulfanyl-substituted alkyl, N-alkylated amino alkyl, and the like. Examples of heteroaryl substituents include furanyl, thiofuranyl, pyrrolyl, pyrrolidinyl, isoxazolyl, benzoxadiazolyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc. Heterocyclic group may also include a substituent group that is heteroatom-containing fusion rings formed between an aromatic ring and an aliphatic ring, for example, a fused ring formed between morpholine and phenyl with the two rings sharing two carbon atoms. The heteroatom may be unsubstituted or substituted with alkyl or aryl.

The terms “cyclic” and “ring” refer to alicyclic or aromatic groups that may or may not be substituted and/or heteroatom containing, and that may be monocyclic, bicyclic, or polycyclic. The term “alicyclic” is used in the conventional sense to refer to an aliphatic cyclic moiety, as opposed to an aromatic cyclic moiety, and may be monocyclic, bicyclic or polycyclic. In one embodiment, the bicyclic or polycyclic ring may be fused ring. The fusion of the ring may be across a bond between two atoms, i.e. two cyclic rings share one bond or two atoms, for example, a decalin; the fusion of the ring may be across a sequence of atoms, i.e. two cyclic rings share three or more atoms, for example a norbornane.

By “substituted” as in “substituted alkyl,” “substituted aryl,” and the like, as alluded to in some of the aforementioned definitions, is meant that in the alkyl, aryl, or other moiety, at least one hydrogen atom bound to a carbon (or other) atom is replaced with one or more non-hydrogen substituents. Examples of such substituents include, without limitation: halo, furanyl, thifuranyl, pyrrolyl, pyridinyl, indolyl, isoxazolyl, benzoxadiazolyl, hydroxyl, sulfhydryl, C₁-C₂₄ alkoxy, C₂-C₂₄ alkenyloxy, C₂-C₂₄ alkynyloxy, C₅-C₂₄ aryloxy, C₆-C₂₄ aryl alkoxy, C₆-C₂₄ alkyl aryloxy, acyl (including C₂-C₂₄ alkylcarbonyl (—CO-alkyl) and C₆-C₂₄ arylcarbonyl (—CO-aryl)), acyloxy (—O-acyl, including C₂-C₂₄ alkylcarbonyloxy (—O—CO-alkyl) and C₆-C₂₄ arylcarbonyloxy (—O—CO-aryl)), C₂-C₂₄ alkoxycarbonyl (−(CO)—O-alkyl), C₆-C₂₄ aryloxycarbonyl (−(CO)—O-aryl), halocarbonyl (—CO)—X where X is halo), C₂-C₂₄ alkylcarbonato (—O—(CO)—O-alkyl), C₆-C₂₄ arylcarbonato (—O—(CO)—O-aryl), carboxy (—COOH), carboxylato (—COO⁻), carbamoyl (−(CO)—NH₂), mono-(C₁-C₂₄ alkyl)-substituted carbamoyl (−(CO)—NH(C₁-C₂₄ alkyl)), di-(C₁-C₂₄ alkyl)-substituted carbamoyl (−(CO)—N(C₁-C₂₄ alkyl)₂), mono-(C₅-C₂₄ aryl)-substituted carbamoyl (−(CO)—NH-aryl), di-(C₅-C₂₄ aryl)-substituted carbamoyl (−(CO)—N(C₅-C₂₄ aryl)₂), di-N—(C₁-C₂₄ alkyl), N—(C₅-C₂₄ aryl)-substituted carbamoyl, thiocarbamoyl (−(CS)—NH₂), mono-(C₁-C₂₄ alkyl)-substituted thiocarbamoyl (−(CO)—NH(C₁-C₂₄ alkyl)), di-(C₁-C₂₄ alkyl)-substituted thiocarbamoyl (−(CO)—N(C₁-C₂₄ alkyl)₂), mono-(C₅-C₂₄ aryl)-substituted thiocarbamoyl (−(CO)—NH-aryl), di-(C₅-C₂₄ aryl)-substituted thiocarbamoyl (−(CO)—N(C₅-C₂₄ aryl)₂), di-N—(C₁-C₂₄ alkyl), N—(C₅-C₂₄ aryl)-substituted thiocarbamoyl, carbamido (—NH—(CO)—NH₂), cyano(—C≡N), cyanato (—O—C≡N), thiocyanato (—S—C≡N), formyl (−(—CO)—H), thioformyl (−(CS)—H), amino (—NH₂), mono-(C₁-C₂₄ alkyl)-substituted amino, di-(C₁-C₂₄ alkyl)-substituted amino, mono-(C₅-C₂₄ aryl)-substituted amino, di-(C₅-C₂₄ aryl)-substituted amino, C₂-C₂₄ alkylamido (—NH—(CO)-alkyl), C₆-C₂₄ arylamido (—NH—(CO)-aryl), imino (—CR═NH where R=hydrogen, C₁-C₂₄ alkyl, C₅-C₂₄ aryl, C₆-C₂₄ alkaryl, C₆-C₂₄ aralkyl, etc.), C₂-C₂₀ alkylimino (—CR═N(alkyl), where R=hydrogen, C₁-C₂₄ alkyl, C₅-C₂₄ aryl, C₆-C₂₄ alkaryl, C₆-C₂₄ aralkyl, etc.), arylimino (—CR═N(aryl), where R=hydrogen, C₁-C₂₀ alkyl, C₅-C₂₄ aryl, C₆-C₂₄ alkaryl, C₆-C₂₄ aralkyl, etc.), nitro (—NO₂), nitroso (—NO), sulfo (—SO₂—OH), sulfonato (—SO₂—O⁻), C₁-C₂₄ alkylsulfanyl (—S-alkyl; also termed “alkylthio”), C₅-C₂₄ arylsulfanyl (—S-aryl; also termed “arylthio”), C₁-C₂₄ alkylsulfinyl (−(SO)-alkyl), C₅-C₂₄ arylsulfinyl (−(SO)-aryl), C₁-C₂₄ alkylsulfonyl (—SO₂-alkyl), C₅-C₂₄ arylsulfonyl (—SO₂-aryl), boryl (—BH₂), borono (—B(OH)₂), boronato (—B(OR)₂ where R is alkyl or other hydrocarbyl), phosphono (—P(O)(OH)₂), phosphonato (—P(O)(O⁻)₂), phosphinato (—P(O)(O⁻)), phospho (—PO₂), phosphino (—PH₂), silyl (—SiR₃ wherein R is hydrogen or hydrocarbyl), and silyloxy (—O-silyl); and the hydrocarbyl moieties C₁-C₂₄ alkyl (preferably C₁-C₁₂ alkyl, more preferably C₁-C₆ alkyl), C₂-C₂₄ alkenyl (preferably C₂-C₁₂ alkenyl, more preferably C₂-C₆ alkenyl), C₂-C₂₄ alkynyl (preferably C₂-C₁₂ alkynyl, more preferably C₂-C₆ alkynyl), C₅-C₂₄ aryl (preferably C₅-C₁₄ aryl), C₆-C₂₄ alkaryl (preferably C₆-C₁₆ alkaryl), and C₆-C₂₄ aralkyl (preferably C₆-C₁₆ aralkyl).

In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated.

In the molecular structures herein, the use of bold and dashed lines to denote particular conformation of groups follows the IUPAC convention. A bond indicated by a broken line indicates that the group in question is below the general plane of the molecule as drawn, and a bond indicated by a bold line indicates that the group at the position in question is above the general plane of the molecule as drawn.

### Tetrahydropyranonaphthyridine Compounds of the Invention:

The invention relates to a compound of formula (III) or (IV):

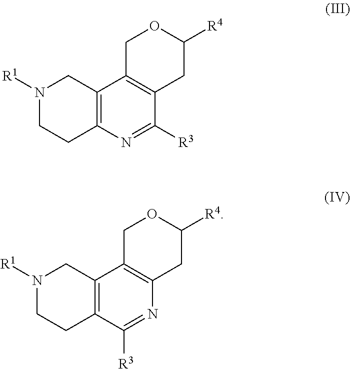

Formulas (III) and (IV) contain asymmetric carbon atoms and hence can exist as stereoisomers, both enantiomers and diastereomers. One of ordinary skill in the art will recognize that the preparation of all possible stereoisomers of formulas (III) and IV may be made by adaption of the preparation methods that are enclosed in this application. The examples provided herein disclose particular isomers and the bioactivity of these isomers, for example those isomers of formulas (III) and (IV) shown as in formulas (III-A) and (IV-A). Other stereoisomers of formulas (III) and (IV) are considered to fall within the scope of the invention.

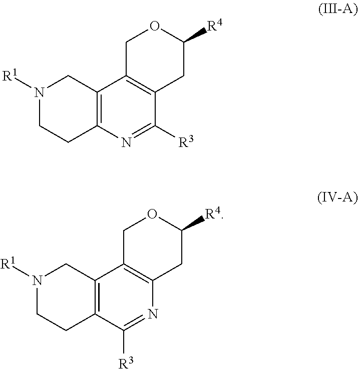

In formulas (III) and (IV), R¹ represents —(CH₂)ₘR⁶, —C(O)N(H)R⁶, —C(O)R⁶, or —S(O)₂R⁶, wherein m is 0-6. In each occurrence of the above formulas represented by R¹, R⁶ is independently a substituted or unsubstituted group selected from the group consisting of alkyl, cycloalkyl, alkenyl, cycloalkenyl, aryl, alkoxy and aryloxy. Alternatively, R⁶ may be substituted or unsubstituted heterocyclic ring containing one or more heteroatoms. The heteroatoms may be N, S, or O. Exemplary heterocyclic rings suitable for R⁶ include, but not limited to furanyl, thiofuranyl, pyrrolyl, pyridinyl, indolyl, imidazolyl, isoxazolyl, and benzoxadiazolyl. In some occurrence, R⁶ may also be a substituted or unsubstituted fusion ring, wherein the fusion forms between aromatic rings or between aliphatic rings or between an aromatic ring and an aliphatic ring. The fusion ring defined here may or may not contain heteroatoms. For example, R⁶ may be a fused ring formed between morpholine and phenyl with two rings sharing two carbon atoms, the N atom of morpholine may be unsubstituted or substituted with alkyl or aryl; R⁶ may be a fused ring formed between a cycloalkyl and cycloalkenyl with the two rings sharing two or more carbon atoms.

In one preferred embodiment, R′ represents —(CH₂)ₘR⁶, wherein m is 0-6, preferably m is 1-3, and most preferably m is 1. R⁶ is a substituted or unsubstituted group selected from the group consisting of alkenyl, cycloalkenyl, aryl, and heterocylic ring containing one or more heteroatoms. Preferably, R⁶ represents a substituted C₂-C₁₂ alkenyl, substituted C₅-C₁₀ cycloalkenyl, substituted aryl, or unsubstituted 5-member heterocylic ring containing one or more heteroatoms, wherein the heteroatoms are O, N or S. The substituent cycloalkenyl may be fused with a cycloalkyl ring, with two rings sharing two or more carbons.

In some preferred embodiments, the substituent group R⁶ defined above is further substituted with one or more moieties selected from the group consisting of alkyl, alkoxy and aryloxy. Preferrably, the substituted alkenyl or cycloalkenyl is an alkenyl or cycloalkenyl group substituted with one or more alkyl groups; and the substituted aryl is a phenyl group substituted with one or more alkoxy or aryloxy groups. For example, R⁶ may be selected from the group consisting of

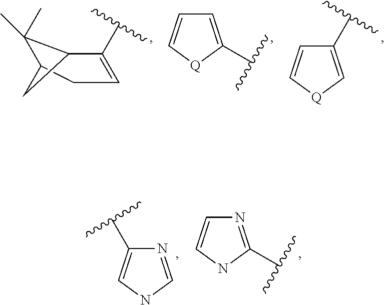

wherein Q is O, S, or N.

In one preferred embodiment, R¹ represents —C(O)N(H)R⁶. R⁶ is a substituted or unsubstituted group selected from the group consisting of alkyl, alkenyl, aryl, and heterocylic ring containing one or more heteroatoms. Preferably, R⁶ represents a substituted C₁-C₆ alkyl, unsubstituted C₂-C₆ alkenyl, substituted aryl, or unsubstituted 5-member heterocylic ring containing one or more heteroatoms, wherein the heteroatoms are O, N or S.

In some preferred embodiments, the substituent group R⁶ defined above is further substituted with one or more moieties selected from the group consisting of alkyl, phenyl, alkoxy, aryloxy, cyano, indolyl, furanyl, thiofuranyl, pyrrolyl, imidazolyl, and —C(O)OR⁸, wherein R⁸ is hydrogen, or substituted or unsubstituted C₁-C₆ alkyl. Preferrably, the substituted alkyl is an alkyl group substituted with one or more moieties selected from the group consisting of alkyl, phenyl, indolyl, furanyl, thiofuranyl, pyrrolyl, imidazolyl, and —C(O)OR⁸, wherein R⁸ is hydrogen, or substituted or unsubstituted C₁-C₆ alkyl; and the substituted aryl is a phenyl group substituted with one or more moieties selected from the group consisting of alkoxy, aryloxy, and cyano. For example, R⁶ may be selected from the group consisting of

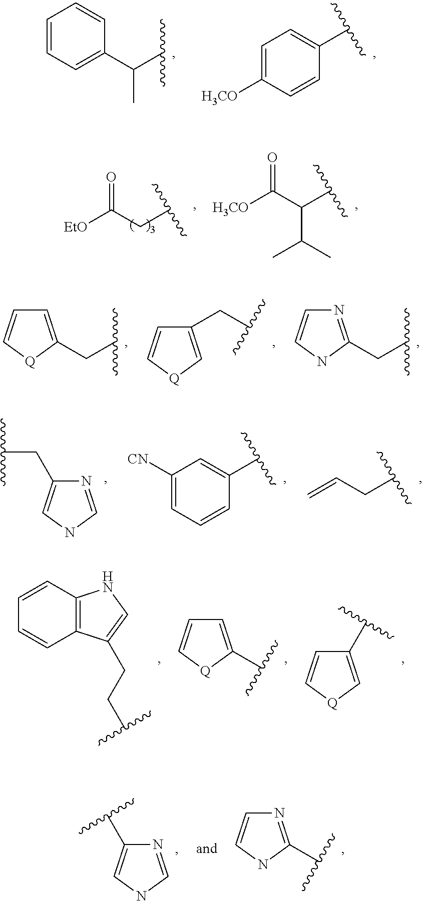

wherein Q is O, S, or N.

In another preferred embodiment, R¹ represents —C(O)R⁶. R⁶ is a substituted or unsubstituted group selected from the group consisting of alkyl, cycloalkyl, aryl, and heterocylic ring containing one or more heteroatoms. Preferably, R⁶ represents a substituted C₁-C₆ alkyl, unsubstituted C₃-C₈ cycloalkyl, substituted aryl, or unsubstituted 5- or 6-member heterocylic ring containing one or more heteroatoms, wherein the heteroatoms are O, N or S.

In some preferred embodiments, the substituent group R⁶ defined above is further substituted with one or more moieties selected from the group consisting of alkyl; halo, alkoxy, aryloxy, phenyl, furanyl, thiofuranyl, pyrrolyl, and imidazolyl. These substituted groups on R⁶ may be further optionally substituted with one or more alkoxy or aryloxy groups. Preferrably, the substituted alkyl is an alkyl group substituted with one or more moieties selected from the group consisting of alkoxy, aryloxy, phenyl, furanyl, thiofuranyl, pyrrolyl, and imidazolyl, which is further optionally substituted with one or more alkoxy or aryloxy groups; and the substituted aryl is a phenyl group substituted with one or more alkyl or halo groups. For example, R⁶ may be selected from the group consisting of methoxyethyl,

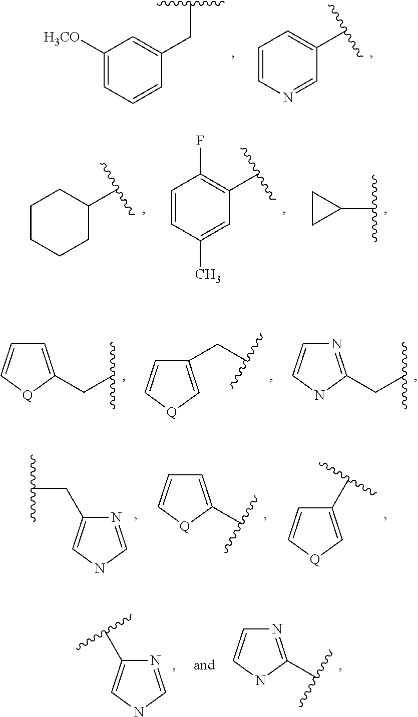

wherein Q is O, S, or N.

In yet another preferred embodiment, R¹ represents —S(O)₂R⁶. R⁶ is a substituted or unsubstituted group selected from the group consisting of alkyl, aryl, heterocylic ring containing one or more heteroatoms, and fused ring formed between a cyclic ring and a heterocyclic ring containing one or more heteroatoms. The substituent aryl may be fused with a heteroatom containing cycloalkyl ring, with two rings sharing two or more carbons. Preferrably, R⁶ represents independently for each occurrence an unsubstituted C₁-C₆ alkyl, substituted aryl, substituted or unsubstituted 5-member heterocylic ring containing one or more heteroatoms, or substituted fused ring formed between a cyclic ring and a heterocyclic ring containing one or more heteroatoms, wherein the heteroatoms are O, N or S.

In some preferred embodiments, the substituent group R⁶ defined above is further substituted with one or more moieties selected from the group consisting of alkyl, halo, cyano, alkoxy, and

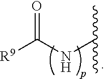

Preferrably, the substituted aryl is a phenyl group substituted with one or more moieties selected from the group consisting of alkyl, cyano and

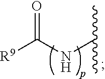

the substituted 5-member heterocylic ring is

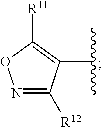

and the substituted fused ring is

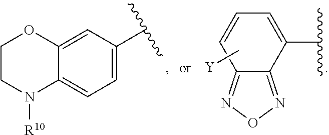

R⁹, R¹⁰, R¹¹ and R¹² are each independently alkyl or alkoxy; p is 0 or 1; and Y is a halide. For example, R⁶ may be selected from the group of n-butyl,

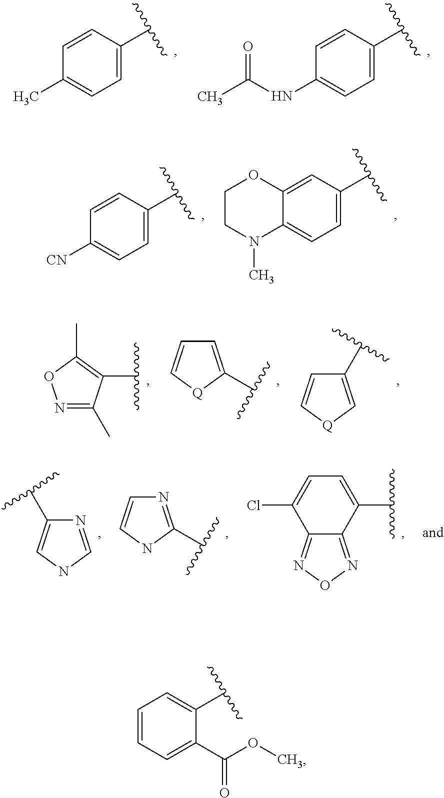

wherein Q is O, S, or N.

In formulas (III) and (IV), R³ and R⁴ each independently represents a substituted or unsubstituted group selected from the group consisting of alkyl, aryl, and heterocylic ring containing one or more heteroatoms. The heterocyclic ring for R³ or R⁴ may be aromatic or aliphatic, and the heteroatoms on the heterocyclic ring are preferably N or O. R³ is preferably an unsubstituted C₁-C₆ alkyl, unsubstituted phenyl, substituted C₁-C₆ alkyl, or substituted phenyl. Preferably, the substituted alkyl or phenyl group is an alkyl or phenyl group substituted with one or more moieties selected from the group consisting of alkyl, alkoxy and aryloxy. Examples of suitable functional groups of R³ include, but not limited to, n-butyl, phenyl,

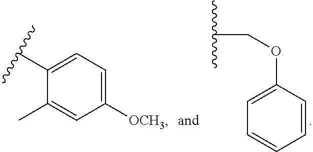

R⁴ is preferably an unsubstituted C₁-C₆ alkyl, unsubstituted phenyl, or substituted C₁-C₆ alkyl, wherein the substituted alkyl group is an alkyl group substituted with one or more moieties selected from the group consisting of alkyl, alkoxy and aryloxy, which may be further optionally substituted with one or more alkoxy or aryloxy groups. Examples of suitable functional groups of R⁴ include, but not limited to, methyl, phenyl and

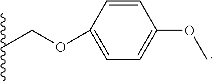

Preferred compounds of the invention include those represented by formula III, with the variables and preferred embodiments of formula (III) defined as above. More preferred compounds of the invention include the following compounds of formulas (IIIa), (IIIb), and (IIIc):

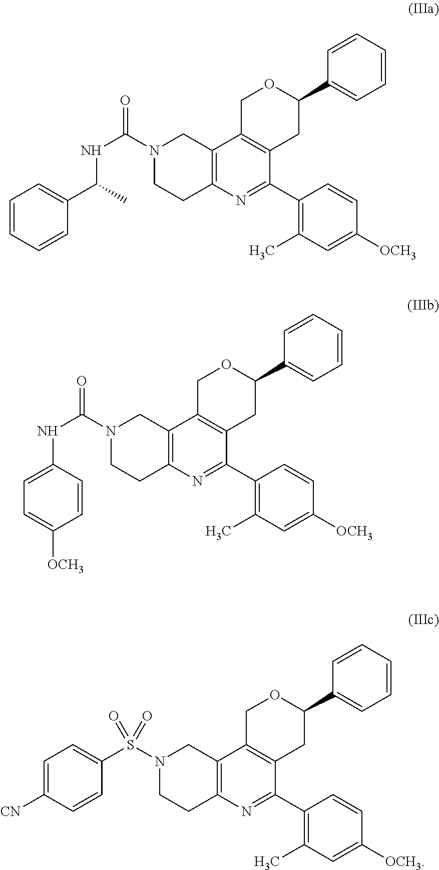

### Tetrahydropyranonaphthyridine Derivatives and Analogues

The invention also relates to a compound of formula (I) or (II):

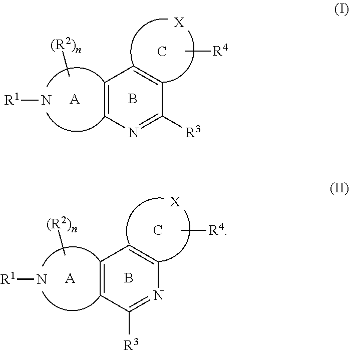

In formulas (I) and (II), A and C are each independently a 5-7 member heterocyclic ring. X on the C— ring may be O, S, S(O), S(O)₂, or NR^{S}, where R⁵ is a substituted or unsubstituted alkyl or aryl. The nitrogen atoms and the variable X may be at any position in the 5-7 heterocyclic A- and C-ring, respectively.

R¹ may be aromatic group or represent formulas —(CH₂)ₘR⁶, —C(O)N(H)R⁶, —C(O)R⁶, or —S(O)₂R⁶. When R¹ is aromatic, it is typically although not necessarily composed of one or two aromatic rings, which may or may not be substituted. For example, R¹ may be phenyl, substituted phenyl, biphenyl, substituted biphenyl, or the like. When R¹ represents formulas —(CH₂)ₘR⁶, —C(O)N(H)R⁶, —C(O)R⁶, or —S(O)₂R⁶, m is 0-6, and R⁶ represents independently for each occurrence a substituted or unsubstituted group selected from the groups of alkyl, cycloalkyl, alkenyl, cycloalkenyl, aryl. Alternatively, R⁶ may be substituted or unsubstituted heterocyclic ring containing one or more heteroatoms. The heteroatoms may be N, S or O. Exemplary heterocyclic rings suitable for R⁶ include, but not limited to furanyl, thiofuranyl, pyrrolyl, pyridinyl, imidazolyl, indolyl, isoxazolyl, and benzoxadiazolyl. In some occurrence, R⁶ may also be a fusion ring, wherein the fusion forms between aromatic rings or between aliphatic rings or between an aromatic ring and an aliphatic ring.

R³ is hydrogen, or a substituted or unsubstituted group selected from the group consisting of alkyl, aryl, heterocylic ring containing one or more heteroatoms. The heterocyclic ring for R³ may be aromatic or aliphatic, and the heteroatoms on the heterocyclic ring are preferably N or O.

The variable n defines the number of substitute groups on the A-ring. The variable n is 0, 1, 2, 3, or 4. The substitutent group on the A-ring is represented by R². Each R² may be the same or different, and is independently H, or a substituted or unsubstituted group selected from the group consisting of alkyl and aryl.

The substituent group on the C— ring is represented by R⁴. When C is a 5-member ring, R⁴ is H; when C is a 6-, or 7-member ring, R⁴ may be hydrogen, or a substituted or unsubstituted group selected from the group consisting of alkyl, aryl, heterocylic ring containing one or more heteroatoms. The heterocyclic ring for R⁴ may be aromatic or aliphatic, and the heteroatoms on the heterocyclic ring are preferably N or O. R⁴ is preferably in an ortho-position to the variable X on the heterocyclic ring, i.e. R⁴ is adjacent to X on the ring and may be on either side of X.

Exemplary compounds of formulas (I) and (II) are illustrated as in Schemes I-VI below. In the analogues of the Schemes I-VI, X may represent O, S, S(O), S(O)₂, or NR⁸, and each of R¹-R⁸ may be independently a substituted or unsubstituted alkyl or aryl, where substituted or unsubstituted alkyl and aryl are defined in the invention. For example, when attached to nitrogen, R¹, R², and R³ can be —(CH₂)ₘR¹³, —C(O)N(H)R¹³, —C(O)R¹³, —S(O)₂R¹³, wherein m is 0-6; and R¹³ represents independently for each occurrence a substituted or unsubstituted alkyl, substituted or unsubstituted cycloalkyl, substituted or unsubstituted alkenyl, substituted or unsubstituted cycloalkenyl, substituted or unsubstituted aryl, substituted or unsubstituted alkoxy, substituted or unsubstituted aryloxy, substituted or unsubstituted heterocylic ring containing one or more heteroatoms, or substituted or unsubstituted fused ring formed between two or more cyclic rings or heteroatom-containing cyclic rings. Each of R⁵ and R⁶ can be independently a substituted or unsubstituted alkyl, substituted or unsubstituted aryl, or substituted or unsubstituted heterocylic ring containing one or more heteroatom.

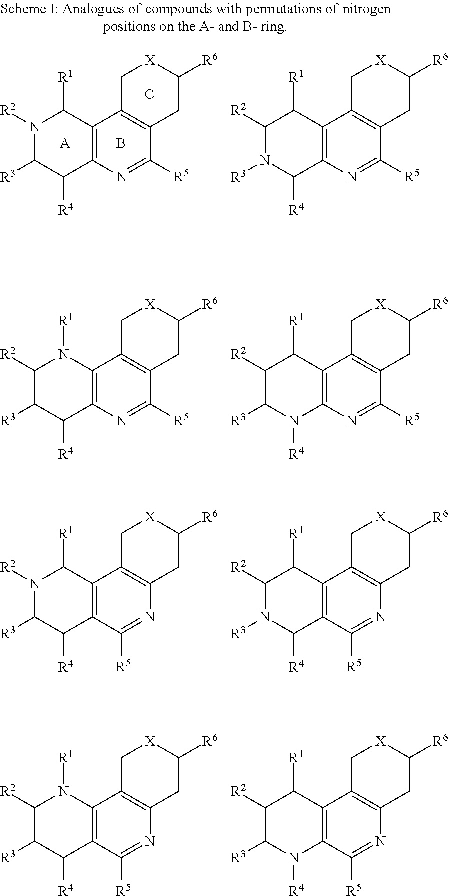

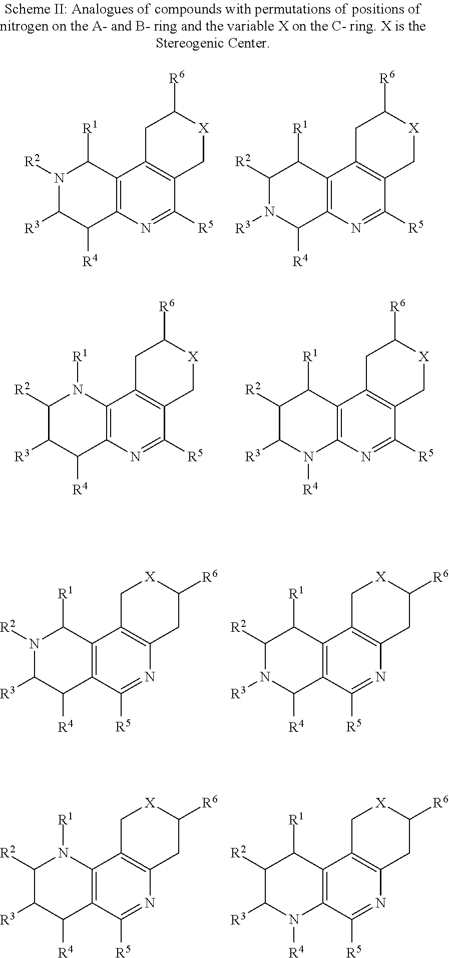

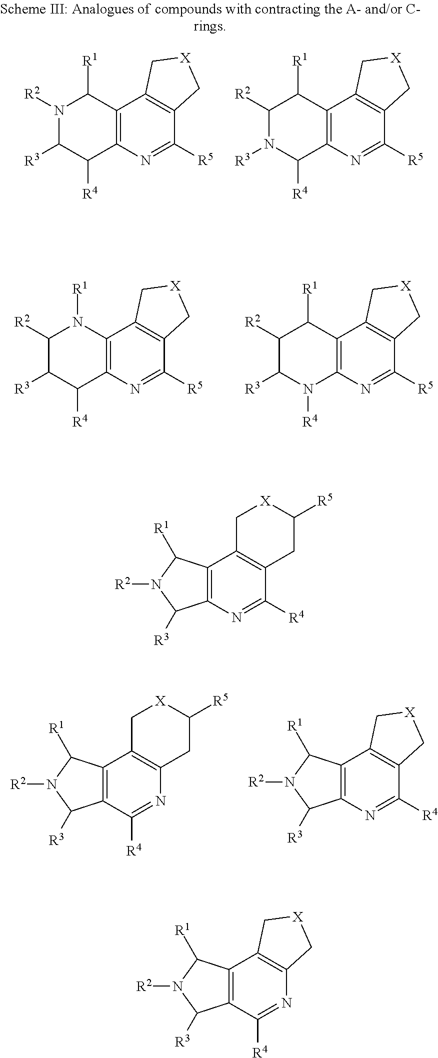

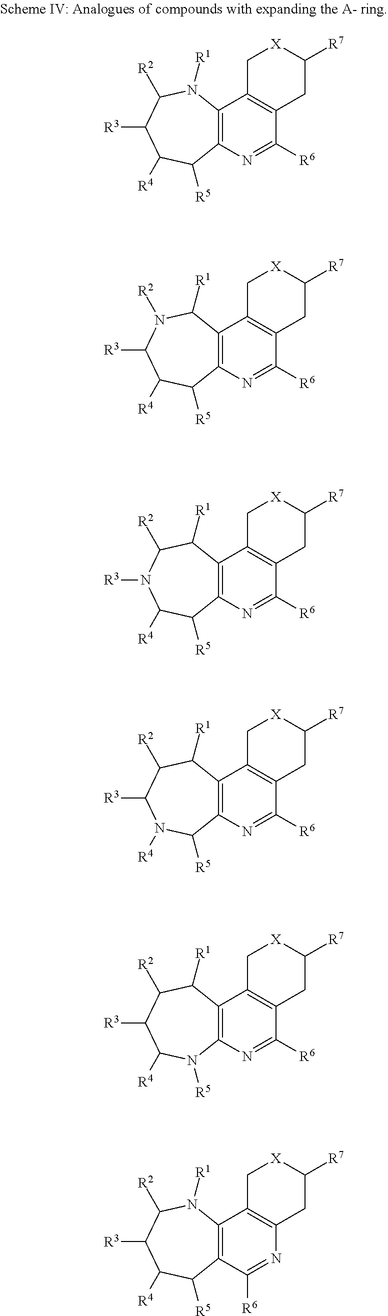

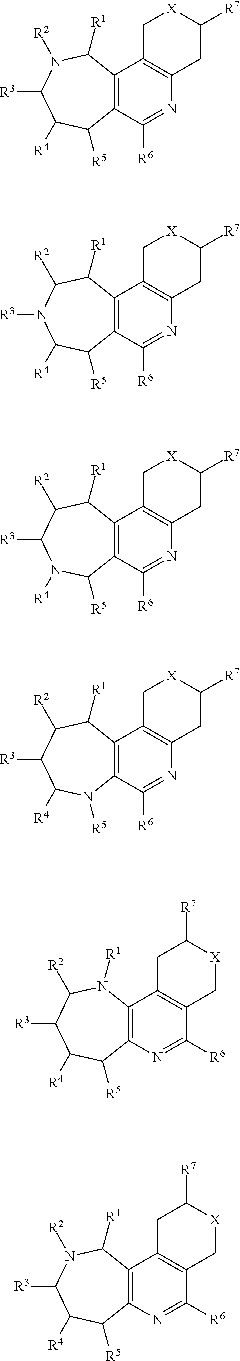

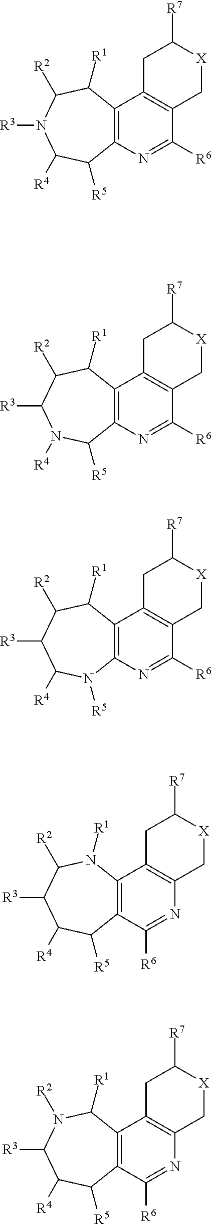

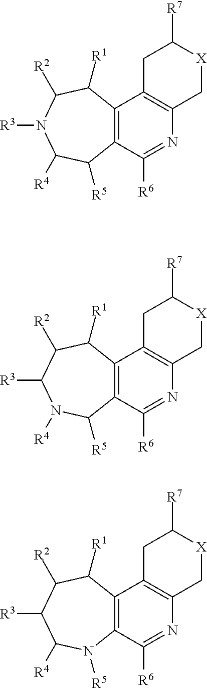

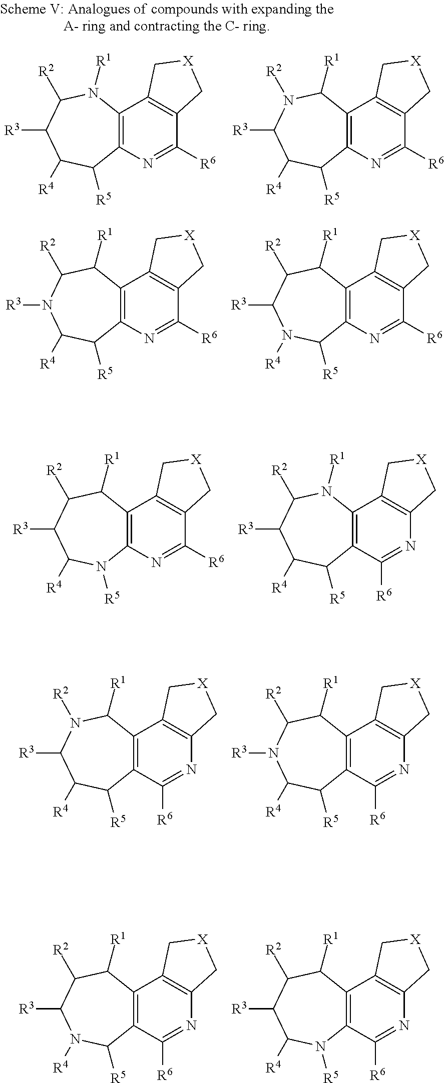

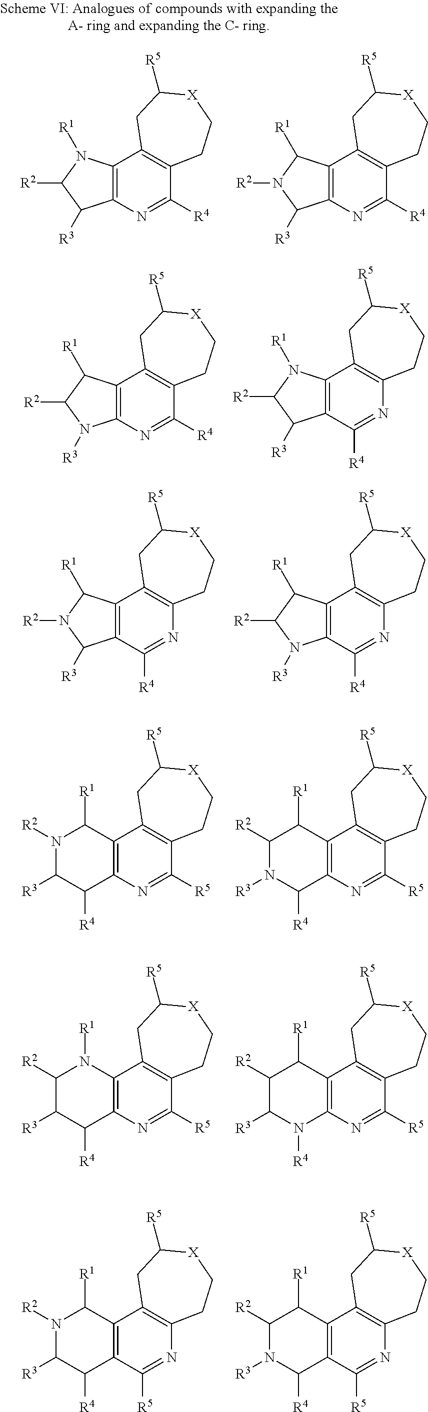

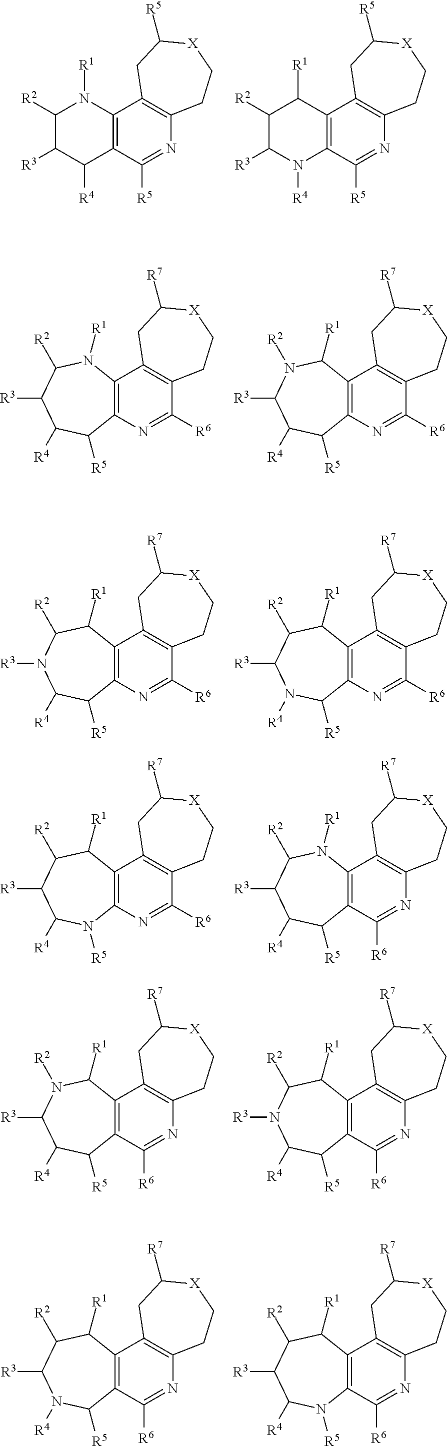

Preferred compounds of the invention include those represented by formula (I), with the variables and preferred embodiments of formula (I) defined as above.

The compounds of formula (I), (II), (III) or (IV) may contain one or more asymmetric centers and thus giving rise to enantiomers, diastereomers, and other stereoisomeric forms that may be defined, in terms of absolute stereochemistry, as (R)— or (S)— or as α or β, Included herein are all such possible isomers, as well as their racemic and optically pure forms.

Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., “Enantiomers, Racemates, and Resolutions”, John Wiley & Sons (1981), content of which is herein incorporated by reference. When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

The compounds of formula (I), (II), (III) or (IV) also include pharmaceutically acceptable salts thereof. As used herein, the term “pharmaceutically-acceptable salts” refers to the conventional nontoxic salts or quaternary ammonium salts of the compounds described herein, e.g., from non-toxic organic or inorganic acids. These salts can be prepared in situ in the administration vehicle or the dosage form manufacturing process, or by separately reacting a purified compound in its free base or acid form with a suitable organic or inorganic acid or base, and isolating the salt thus formed during subsequent purification. Conventional nontoxic salts include those derived from inorganic acids such as sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. See, for example, Berge et al., “Pharmaceutical Salts”, J. Pharm. Sci. 66:1-19 (1977), content of which is herein incorporated by reference in its entirety.

In some embodiments of the aspects described herein, representative pharmaceutically acceptable salts include the hydrobromide, hydrochloride, sulfate, bisulfate, phosphate, nitrate, acetate, succinate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like.

Prodrugs of the compounds of formula (I), (II), (III) or (IV) also fall within the scope of the invention. As used herein, a “prodrug” refers to compounds that can be converted via some chemical or physiological process (e.g., enzymatic processes and metabolic hydrolysis) to compound of formula (I), (II), (III) or (IV). Thus, the term “prodrug” also refers to a precursor of a biologically active compound that is pharmaceutically acceptable. A prodrug may be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like. See Harper, “Drug Latentiation” in Jucker, ed. Progress in Drug Research 4:221-294 (1962); Morozowich et al, “Application of Physical Organic Principles to Prodrug Design” in E. B. Roche ed. Design of Biopharmaceutical Properties through Prodrugs and Analogs, APHA Acad. Pharm. Sci. 40 (1977); Bioreversible Carriers in Drug in Drug Design, Theory and Application, E. B. Roche, ed., APHA Acad. Pharm. Sci. (1987); Design of Prodrugs, H. Bundgaard, Elsevier (1985); Wang et al. “Prodrug approaches to the improved delivery of peptide drug” in Curr. Pharm. Design. 5(4):265-287 (1999); Pauletti et al. (1997) Improvement in peptide bioavailability: Peptidomimetics and Prodrug Strategies, Adv. Drug. Delivery Rev. 27:235-256; Mizen et al. (1998) “The Use of Esters as Prodrugs for Oral Delivery of beta-Lactam antibiotics,” Pharm. Biotech. 11:345-365; Gaignault et al. (1996) “Designing Prodrugs and Bioprecursors I. Carrier Prodrugs,” Pract. Med. Chem. 671-696; Asgharnejad, “Improving Oral Drug Transport”, in Transport Processes in Pharmaceutical Systems, G. L. Amidon, P. I. Lee and E. M. Topp, Eds., Marcell Dekker, p. 185-218 (2000); Balant et al., “Prodrugs for the improvement of drug absorption via different routes of administration”, Eur. J. Drug Metab. Pharmacokinet., 15(2): 143-53 (1990); Balimane and Sinko, “Involvement of multiple transporters in the oral absorption of nucleoside analogues”, Adv. Drug Delivery Rev., 39(1-3): 183-209 (1999); Browne, “Fosphenyloin (Cerebyx)”, Clin. Neuropharmacol. 20(1): 1-12 (1997); Bundgaard, “Bioreversible derivatization of drugs—principle and applicability to improve the therapeutic effects of drugs”, Arch. Pharm. Chemi 86(1): 1-39 (1979); Bundgaard H. “Improved drug delivery by the prodrug approach”, Controlled Drug Delivery 17: 179-96 (1987); Bundgaard H. “Prodrugs as a means to improve the delivery of peptide drugs”, Arfv. Drug Delivery Rev. 8(1): 1-38 (1992); Fleisher et al. “Improved oral drug delivery: solubility limitations overcome by the use of prodrugs”, Arfv. Drug Delivery Rev. 19(2): 115-130 (1996); Fleisher et al. “Design of prodrugs for improved gastrointestinal absorption by intestinal enzyme targeting”, Methods Enzymol. 112 (Drug Enzyme Targeting, Pt. A): 360-81, (1985); Farquhar D, et al., “Biologically Reversible Phosphate-Protective Groups”, Pharm. Sci., 72(3): 324-325 (1983); Freeman S, et al., “Bioreversible Protection for the Phospho Group: Chemical Stability and Bioactivation of Di(4-acetoxy-benzyl) Methylphosphonate with Carboxyesterase,” Chem. Soc., Chem. Commun., 875-877 (1991); Friis and Bundgaard, “Prodrugs of phosphates and phosphonates: Novel lipophilic alphaacyloxyalkyl ester derivatives of phosphate- or phosphonate containing drugs masking the negative charges of these groups”, Eur. J. Pharm. Sci. 4: 49-59 (1996); Gangwar et al., “Pro-drug, molecular structure and percutaneous delivery”, Des. Biopharm. Prop. Prodrugs Analogs, [Symp.] Meeting Date 1976, 409-21. (1977); Nathwani and Wood, “Penicillins: a current review of their clinical pharmacology and therapeutic use”, Drugs 45(6): 866-94 (1993); Sinhababu and Thakker, “Prodrugs of anticancer agents”, Adv. Drug Delivery Rev. 19(2): 241-273 (1996); Stella et al., “Prodrugs. Do they have advantages in clinical practice?”, Drugs 29(5): 455-73 (1985); Tan et al. “Development and optimization of anti-HIV nucleoside analogs and prodrugs: A review of their cellular pharmacology, structure-activity relationships and pharmacokinetics”, Adv. Drug Delivery Rev. 39(1-3): 117-151 (1999); Taylor, “Improved passive oral drug delivery via prodrugs”, Adv. Drug Delivery Rev., 19(2): 131-148 (1996); Valentino and Borchardt, “Prodrug strategies to enhance the intestinal absorption of peptides”, Drug Discovery Today 2(4): 148-155 (1997); Wiebe and Knaus, “Concepts for the design of anti-HIV nucleoside prodrugs for treating cephalic HIV infection”, Adv. Drug Delivery Rev.: 39(1-3):63-80 (1999); Waller et al., “Prodrugs”, Br. J. Clin. Pharmac. 28: 497-507 (1989), content of all of which is herein incorporated by reference in its entirety.

### Design of the Synthetic Route of the Compound Library

The compounds of the invention possess unusual and unique structures. As discussed in the above embodiments, some exemplary compounds suitable for the invention include those belonging to an annulated tetrahydro-1,6-naphthyridine class formally known as 2,3,4,7,8,10-hexahydro-1H-pyrano[4,3-c][1,6]naphthyridines.

The invention provides a new approach to synthesize tetrahydronaphthyridines using a transition metal catalyzed [2+2+2] cyclization of diynes with nitriles. The basic structures of these compounds are extremely rare, and represent a new chemotype with drug-like features: relatively low molecular weight, basic nitrogens for hydrogen bonding, and suitable solubility in both lipophilic and aqueous phases. The interest was to explore a different type of chemistry to prepare tetrahydronaphthyridines including, 5,6,7,8-tetrahydro-1,6-naphthyridines and 1,2,3,4-tetrahydro-1,5-naphthyridines. The preparation of the target compounds, in particular, the tetrahydro-1,6-naphthyridines, can not be prepared by the earlier available chemistry, which employed inverse electron demand Diels-Alder cycloadditions. Thus the transition metal catalyzed cyclization was designed, as illustrated in the Examples.

Preparation of the Compounds of the Invention by the Catalyzed Cyclization is straightforward, and easy to accomplish on a large scale. A vast number of structural variations can be produced based on the parent 5,6,7,8-tetrahydro-1,6-naphthyridine core. For example, as illustrated above in Schemes I-VI, the compound of invention include those analogues by variation of the size of A-, and/or C-rings, permutations of the positions of nitrogens and the variable X in the A-, and/or B-, and/or C-rings.

Studies of reaction conditions revealed that cobalt (I) catalysts under microwave promotion were highly successful in the cyclization, readily yielding the desired tetrahydro-1,6-naphthyridines. Both intermolecular and intramolecular cyclizations were examined. The intramolecular reactions produced a series of 2,3; 4,7,8,10-hexahydro-1Hpyrano[4,3-c][1,6]naphthyridines, as well as the A-ring contracted and the A-ring expanded analogues, as described in Schemes I-VI above. Thus the new chemistry designed in this invention not only worked very efficiently, it has also been proved to be highly versatile for the synthesis of a large variety of structural analogues.

### Pharmaceutical Compositions of the Invention

One aspect of the invention relates to pharmaceutical compositions comprising a therapeutically effective amount of a compound as in the formula (I), (II), (III) or (IV), defined as above, according to the invention and a pharmaceutically acceptable carrier, (also known as a pharmaceutically acceptable excipient).

The compounds of formula (I), (II), (III) or (IV) are therapeutically useful for the treatment or prevention of disease states associated with tuberculosis or bacteria infection caused by tuberculosis, especially Mycobacterium tuberculosis. The compounds of formula (I), (II), (III) or (IV) possess therapeutic activity against Mycobacterium tuberculosis. Pharmaceutical compositions for the treatment of those disease states comprises a therapeutically effective amount of a compound of formula (I), (II), (III) or (IV) according to the invention to inhibit, or regulate the growth of M. tuberculosis as appropriate for treatment of a patient with the particular disease. A pharmaceutical composition of the invention may be any pharmaceutical form which comprises the compound of formula (I), (II), (III) or (IV) according to the invention. The pharmaceutical composition may be, for example, a tablet, capsule, pills, powders, granules, liquid suspension, injectable, topical, or transdermal. The pharmaceutical composition may comprise active compounds mixing with at least one inert, pharmaceutically acceptable excipient (also known as a pharmaceutically acceptable carrier). The pharmaceutical compositions generally comprise about 1% to about 99% by weight of a compound of formula (I), (II), (III) or (IV) of the invention and 99% to 1% by weight of a suitable pharmaceutical excipient. In one example, the composition will be between about 5% and about 75% by weight of a compound of formula (I), (II), (III) or (IV) of the invention with the rest being suitable pharmaceutical excipients or other adjuvants, as discussed below.

A “therapeutically effective amount” of a compound of formula (I), (II), (III) or (IV), according to the invention, to inhibit, or regulate the growth of M. tuberculosis refers to an amount sufficient to treat a patient suffering from any of a variety of diseases or bacteria infection associated with tuberculosis. The actual amount required for treatment of any particular patient will depend upon a variety of factors including the disease state being treated and its severity; the specific pharmaceutical composition employed; the age, body weight, general health, sex and diet of the patient; the mode of administration; the time of administration; the route of administration; and the rate of excretion of the crystalline form of a compound of formula (I), (II), (III) or (IV) according to the invention; the duration of the treatment; any drugs used in combination or coincidental with the specific compound employed; and other such factors well known in the medical arts. These factors are discussed in Goodman and Gilman's “The Pharmacological Basis of Therapeutics”, Tenth Edition, A. Gilman, J. Hardman and L. Limbird, eds., McGraw-Hill Press, 155-173, 2001, which is incorporated herein by reference. The compounds of formula (I), (II), (III) or (IV) according to the invention and pharmaceutical compositions comprising them may be used in combination with anti-bacterial infection, anti-tuberculosis or other agents that are generally administered to a patient being treated for anti-bacteria infection or anti-tuberculosis. They may also be co-formulated with one or more of such agents in a single pharmaceutical composition.

Depending on the type of pharmaceutical composition, the pharmaceutically acceptable carrier may be chosen from any one or a combination of carriers known in the art. The choice of the pharmaceutically acceptable carrier depends upon the pharmaceutical form and the desired method of administration to be used. For a pharmaceutical composition of the invention, that is, one comprising a compound of formula (I), (II), (III) or (IV) of the invention, a carrier should be chosen so as to substantially maintain the stability of the compound of formula (I), (II), (III) or (IV) of the invention. The carrier should not be otherwise incompatible with the compound of formula (I), (II); (III) or (IV) according to the invention, such as by producing any undesirable biological effect or otherwise interacting in a deleterious manner with any other component(s) of the pharmaceutical composition.

The pharmaceutical compositions of the invention may be prepared by methods know in the pharmaceutical formulation art, for example, see Remington's Pharmaceutical Sciences, 18th Ed., (Mack Publishing Company, Easton, Pa., 1990). In a solid dosage forms, the compound of formula (I), (II), (III) or (IV) is admixed with at least one pharmaceutically acceptable excipient such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Pharmaceutically acceptable adjuvants known in the pharmaceutical formulation art may also be used in the pharmaceutical compositions of the invention. These include, but are not limited to, preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. If desired, a pharmaceutical composition of the invention may also comprise minor amounts of auxiliary substances such as wetting or emulsifying agents, pH buffering agents, antioxidants, and the like, such as, for example, citric acid, sorbitan monolaurate, triethanolamine oleate, butylalted hydroxytoluene, etc.

Solid dosage forms as described above can be prepared with coatings and shells, such as enteric coatings and others well known in the art. They may contain pacifying agents, and can also be of such composition that they release the active compound or compounds in a certain part of the intestinal tract in a delayed manner. Examples of embedded compositions that can be used are polymeric substances and waxes. The active compounds can also be in microencapsulated form, if appropriate, with one or more of the above-mentioned excipients.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Administration of a compound of formula (I), (II), (III) or (IV) in pure form or in an appropriate pharmaceutical composition can be carried out via any of the accepted modes of administration or agents for serving similar utilities. Thus, administration can be, for example, orally, nasally, parenterally (intravenous, intramuscular, or subcutaneous), topically, transdermally, intravaginally, intravesically, intracistemally, or rectally, in the form of solid, semi-solid, lyophilized powder, or liquid dosage forms, such as for example, tablets, suppositories, pills, soft elastic and hard gelatin capsules, powders, solutions, suspensions, or aerosols, or the like, preferably in unit dosage forms suitable for simple administration of precise dosages. One preferable route of administration is oral administration, using a convenient dosage regimen that can be adjusted according to the degree of severity of the disease-state to be treated.

For administration by inhalation, the compounds of the invention are conveniently delivered in the form of an aerosol spray presentation from pressurized as powders which may be formulated and the powder compositions may be inhaled with the aid of an insufflation powder inhaler device. One exemplary delivery system for inhalation is a metered dose inhalation aerosol, which may be formulated as a suspension or solution of compound in suitable propellants such as fluorocarbons or hydrocarbons. Because of desirability to directly treat lung and bronchi, aerosol administration is a preferred method of administration. Insufflation is also a desirable method, especially where infection may have spread to ears and other body cavities.

Aerosol formulations can be arranged so that each metered dose or “puff” of aerosol contains from about 1 μg to about 10 mg, preferably about 20 μg to about 5 mg μg of a compound of formula (I), (II), (III) or (IV). Aerosol administration may be once daily or several times daily, for example 2, 3, 4 or 8 times, giving for example 1, 2 or 3 doses each time. Preferably the compound of formula (I), (II), (III) or (IV) is delivered once, twice, or three times daily. The overall daily dose with an aerosol for administration to the lung in the treatment tuberculosis will typically be within the range 10 μg to 100 mg, for example from about 50 μg to about 50 mg, from about 50 μg to about 20 mg, from about 50 μg to about 10 mg, from about 50 μg to about 1 mg. It is to be understood that that the intermediate ranges to the above ranges specified are also contemplated and claimed in the present invention.

The compounds of the invention can be administrated to a subject in combination with a pharmaceutically active agent. Exemplary pharmaceutically active compound include, but are not limited to, those found in Harrison's Principles of Internal Medicine, 13^{th} Edition, Eds. T. R. Harrison et al. McGraw-Hill N.Y., NY; Physicians Desk Reference, 50^{th} Edition, 1997, Oradell New Jersey, Medical Economics Co.; Pharmacological Basis of Therapeutics, 8^{th} Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete content of all of which are herein incorporated in its entirety. In some embodiments, pharmaceutically active agent include, but are not limited to, anti-bacterial infection, anti-tuberculosis or other agents that are generally administered to a patient being treated for anti-bacterial infection or anti-tuberculosis. In some embodiments, pharmaceutically active agent is an antibiotic agent.

The term “antibiotic” is art recognized and refers to any compound known to one of ordinary skill in the art that will inhibit the growth of, or kill, bacteria. As used herein the term “antibiotic” includes antimicrobial agents synthesized by an organism in nature and isolated from this natural source, and chemically synthesized drugs. Antibiotics include, but are not limited to, polyether ionophore such as monensin and nigericin; macrolide antibiotics such as erythromycin and tylosin; aminoglycoside antibiotics such as streptomycin and kanamycin; beta-lactam antibiotics such as penicillin and cephalosporin; and polypeptide antibiotics such as subtilisin and neosporin. Semi-synthetic derivatives of antibiotics, and antibiotics produced by chemical methods are also encompassed by this term. Chemically-derived antimicrobial agents such as isoniazid, trimethoprim, quinolones, fluoroquinolones and sulfa drugs are considered antibacterial drugs, and the term antibiotic includes these. It is within the scope of the present invention to include compounds derived from natural products and compounds that are chemically synthesized.

Exemplary antibiotics include, but are not limited to, Penicillin, cephalosporins, vancomycins, bacitracins, macrolides, erythromycins, lincosamides (clindomycin), chloramphenicols, tetracyclines, aminoglycosides, gentamicins, amphotericins, cefazolins, clindamycins, mupirocins, sulfonamides, trimethoprim, rifampicins, metronidazoles, quinolones, novobiocins, polymixins, gramicidins, and any salts or variants thereof. It is understood that it is within the scope of the present invention that the tetracyclines include, but are not limited to, immunocycline, chlortetracycline, oxytetracycline, demeclocycline, methacycline, deoxycycline and minocycline.

A compound described herein and a pharmaceutically active agent can be administrated to the subject in the same pharmaceutical composition or in different pharmaceutical compositions (at the same time or at different times). When administrated at different times, the compound described herein and the pharmaceutically active agent can be administered within 5 minutes, 10 minutes, 20 minutes, 60 minutes, 2 hours, 3 hours, 4, hours, 8 hours, 12 hours, 24 hours of administration of the other Without limitation, when a compound described herein and a pharmaceutically active agent are administered in different pharmaceutical compositions, routes of administration can be different for each.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the compounds described herein. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments of the aspects described herein, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

### Inhibition of Mycobacterial Growth

The compounds of the invention are useful for inhibiting the growth of mycobacterium. Accordingly, the invention provides methods for inhibiting the growth of a mycobaterium. The method comprising contacting a compound of formula (I) or (II) or a salt thereof with the mycobacterium.

The skilled artisan is well aware of the methods for measuring growth of mycobacteria. A common method of detecting growth in mycobacterial cultures involves the use of oxygen-sensitive luminescent compounds. An exemplary method is the microplate Alamar Blue assay (MABA), described in Falzari, et al., Antimicrob. Agents Chemother. 2005, 49, 1447-1454 (2005), content of which is herein incorporated by reference.

The mycobacterium can be contacted with the compounds formaula (I) or II in a cell culture e.g., in vitro, or the compounds can be administrated to a subject, e.g., in vivo. Without wishing to be bound by theory, in vivo methods can be used for treating tuberculosis, bacteria infection caused by tuberculosis, or related diseases, by exploiting the inhibition or modulation of tuberculosis growth, in particular the M. tuberculosis growth.

The term “contacting” or “contact” as used herein in connection with contacting mycobacterium includes subjecting the mycobacterium to an appropriate culture media which comprises the indicated compound of formula (I) or (II). Where the mycobacterium is in vivo, “contacting” or “contact” includes administering the compound of formula (I) or (II) in a pharmaceutical composition to a subject via an appropriate administration route such that compound contacts the mycobacterium in vivo.

For in vivo methods, a therapeutically effective amount of a compound of formula (I) or (II) can be administered to a subject. Methods of administering compounds to a subject are known in the art and easily available to one of skill in the art.

Without limitation, the compound of formula (I) or (II) can inhibits the growth of mycobacterium by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90, %, at least 95%, at least 98%, at least, or 100% (e.g. complete inhibition) as compared to a non-inhibited control.

In some embodiments, the compound of formula (I) or (II) has a minimum inhibitory concentration (MIC) on growth of mycobacterium of less than 100 μM, less than 50 μM, less than 25 μM, less than 20 μM, less than 15 μM, less than 10 μM, less than 5 μM, less than 2.5 μM, or less than 1 μM. “MIC” is an art recognized term and refers to the lowest concentration of a compound that will inhibit the visible growth of a microorganism after overnight incubation. MICs can be determined by agar or broth dilution methods usually following the guidelines of a reference body such as the CLSI, BSAC or EUCAST. There are several commercial methods available, including the well established Etest strips and the Oxoid MICEvaluator method.

### Methods of Treatment

The invention also provides methods for treating tuberculosis, bacteria infection caused by tuberculosis, or related diseases, by exploiting the inhibition or modulation of tuberculosis growth, in particular the M. tuberculosis growth. The method comprises administering to a subject in need of such treatment a therapeutically effective amount of a compound of formula (I) or (II), or a salt thereof:

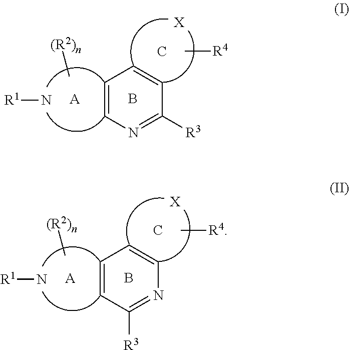

In formulas (I) and (II), A and C are each independently a 5-7 member heterocyclic ring. X on the C— ring may be O, S, S(O), S(O)₂, or NR^{S}, where R⁵ is a substituted or unsubstituted alkyl or aryl. The nitrogen atoms and the variable X may be at any position in the 5-7 heterocyclic A- and C-ring, respectively.

R¹ may be H, alkyl, aromatic group or represent formulas —(CH₂)ₘR⁶, —C(O)N(H)R⁶, —C(O)R⁶, or —S(O)₂R⁶. When R¹ is aromatic, it is typically although not necessarily composed of one or two aromatic rings, which may or may not be substituted. For example, R¹ may be phenyl, substituted phenyl, biphenyl, substituted biphenyl, or the like. When R¹ represents formulas —(CH₂)ₘR⁶, —C(O)N(H)R⁶, —C(O)R⁶, or —S(O)₂R⁶, m is 0-6, and R⁶ represents independently for each occurrence a substituted or unsubstituted group selected from the groups of alkyl, cycloalkyl, alkenyl, cycloalkenyl, aryl. Alternatively, R⁶ may be substituted or unsubstituted heterocyclic ring containing one or more heteroatoms. The heteroatoms may be N, S or O. Exemplary heterocyclic rings suitable for R⁶ include, but not limited to furanyl, thiofuranyl, pyrrolyl, pyridinyl, imidazolyl, indolyl, isoxazolyl, and benzoxadiazolyl. In some occurrence, R⁶ may also be a fusion ring, wherein the fusion forms between aromatic rings or between aliphatic rings or between an aromatic ring and an aliphatic ring.

R³ is hydrogen, or a substituted or unsubstituted group selected from the group consisting of alkyl, aryl, heterocylic ring containing one or more heteroatoms. The heterocyclic ring for R³ may be aromatic or aliphatic, and the heteroatoms on the heterocyclic ring are preferably N or O.

The variable n defines the number of substitute groups on the A-ring. The variable n is 0, 1, 2, 3, or 4. The substitutent group on the A-ring is represented by R². Each R² may be the same or different, and is independently H, or a substituted or unsubstituted group selected from the group consisting of alkyl and aryl.

The substituent group on the C— ring is represented by R⁴. When C is a 5-member ring, R⁴ is H; when C is a 6-, or 7-member ring, R⁴ may be hydrogen, or a substituted or unsubstituted group selected from the group consisting of alkyl, aryl, heterocylic ring containing one or more heteroatoms. The heterocyclic ring for R³ may be aromatic or aliphatic, and the heteroatoms on the heterocyclic ring are preferably N or O. R⁴ is preferably in an ortho-position to the variable X on the heterocyclic ring, i.e. R⁴ is adjacent to X on the ring and may be on either side of X.

Some embodiments of the invention provides methods for treating tuberculosis, bacteria infection caused by tuberculosis, or related diseases, by exploiting the inhibition or modulation of tuberculosis growth, in particular the M. tuberculosis growth. The method comprises administering to a subject in need of such treatment a therapeutically effective amount of a compound of formula (III) or (IV), or a salt thereof:

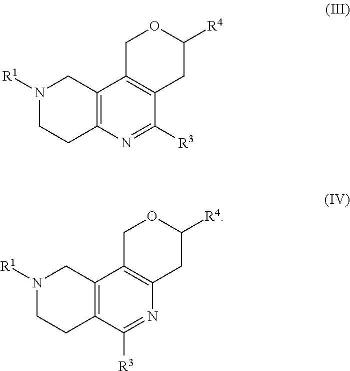

The preferred compounds of formulas (III) and (IV) in the method for treating tuberculosis of the invention include those described in the preferred embodiments defined above for formulas (III) and (IV). More preferred compounds in the method for treating tuberculosis of the invention include the following compounds of formulas (IIIa), (IIIb), and (IIIc):

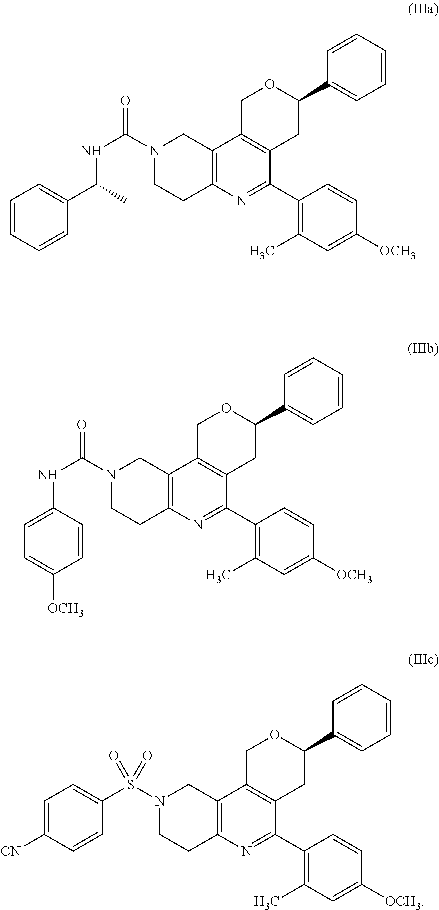

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used as subjects that represent animal models of disorders associated with autoimmune disease or inflammation.

In addition, the methods described herein can be used to treat domesticated animals and/or pets. A subject can be male or female.

The subject to be treated is generally a mammal and most often a human. The disease being treated is generally one discussed above, such as tuberculosis or bacteria infection caused by tuberculosis, in particular M. tuberculosis, or related diseases. As used herein, the term “infection” shall be understood to mean invasion and/or colonisation by a microorganism and/or multiplication of a micro-organism, in particular, a bacterium or a virus, in the respiratory tract of a subject. Such an infection may be unapparent or result in local cellular injury. The infection may be localized, subclinical and temporary or alternatively may spread by extension to become an acute or chronic clinical infection. The infection may also be a latent infection, in which the microorganism is present in a subject, however the subject does not exhibit symptoms of disease associated with the organism. Preferably, the infection is a pulmonary or extra-pulmonary infection by M. tuberculosis. By “pulmonary” infection is meant an infection of the airway of the lung, such as, for example, an infection of the lung tissue, bronchi, bronchioles, respiratory bronchioles, alveolar ducts, alveolar sacs, or alveoli. By “extrapulmonary” is meant outside the lung, encompassing, for example, kidneys, lymph, urinary tract, bone, skin, spinal fluid, intestine, peritoneal, pleural and pericardial cavities.

A subject can be one who has been previously diagnosed with or identified as suffering from tuberculosis, bacterial infection, or related diseases. The subject need not have already undergone treatment or be currently undergoing treatment.

For example, a subject can be diagnosed with tuberculosis based on the symptoms presented by the subject. The classic symptoms of tuberculosis include, a chronic cough with blood-tinged sputum, fever, night sweats, and weight loss. Infection of other organs by M. tuberculosis causes a wide range of symptoms. In some cases diagnosis relies on radiology (commonly chest X-rays), a tuberculin skin test, blood tests, as well as microscopic examination and microbiological culture of bodily fluids.

The compounds the present invention can be administered to subject, in amounts effective to provide the desired tuberculosis inhibitory activity. Since the activity of the compounds and the degree of the desired therapeutic effect vary, the dosage level of the compound employed will also vary. The actual dosage administered will also be determined by such generally recognized factors as the body weight of the patient and the individual hypersensitiveness of the particular patient. For example, oral administration may require a total daily dose of from 1 mg to 2000 mg, while an intravenous dose may only require from 0.01 mg to 100 mg. The total daily dose may be administered in single or divided doses and may, at the physician's discretion, fall outside of the typical range given herein. The unit dosage for a particular patient (man) can vary from as low as about 0.001 mg per kg of body weight, which the practitioner may titrate to the desired effect. A preferred minimum dose for titration is from about from about 0.001 mg/kg to about 500 mg/kg body weight, preferably from about 1 mg/kg to about 350 mg/kg body weight, and more preferably from about 5 mg/kg to about 200 mg/kg body weight. The total dose may be administered in single or divided doses daily or once a week, or twice a week or the like, and may, at the physician's discretion, fall outside of the typical range given herein. A preferred dose is 0.001-50 mg per kg body weight daily, or 1-100 mg once-a-week or twice-a-week.

The therapeutically effective dose can be estimated initially from cell culture assays. A dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay.

The amount of a compound described herein that can be combined with a carrier material to produce a single dosage form will generally be that amount of the compound that produces a therapeutic effect. Generally out of one hundred percent, this amount will range from about 0.01% to 99% of the compound, preferably from about 5% to about 70%, most preferably from 10% to about 30%.

Toxicity and therapeutic efficacy of the compounds described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices, are preferred.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

By “treatment”, “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. For the avoidance of doubt, references herein to “treatment” include references to curative, palliative and prophylactic treatment. In some embodiments, at least one symptom of a disease or disorder is alleviated by at least 5%, at least 10%, at least 20%, at least 30%, at least 40%, or at least 50% or more.

For treatment of tuberculosis, the effect of treatment on a subject can be measured by a reduction in at least one of the symptoms of tuberculosis, e.g., an indication selected from the group consisting of a decrease in fever, a decrease in chronic cough, a reduction in sputum production, a reduction in wheezing, a decrease in night sweats, a reduction in conversion of sputum cultures, and any combinations thereof.

A subject can be one who has been previously diagnosed with or identified as suffering from tuberculosis, bacterial infection, or related diseases and the subject is undergoing treatment with one or more antibiotics.

Effective tuberculosis treatment has been difficult, due to the unusual structure and chemical composition of the mycobacterial cell wall, which makes many antibiotics ineffective and hinders the entry of drugs. Generally, tuberculosis requires longer periods of treatment (around 6 to 24 months) to entirely eliminate mycobacteria from the body. See for example, Centers for Disease Control and Prevention (CDC), Division of Tuberculosis Elimination, Core Curriculum on Tuberculosis: What the Clinician Should Know, 4th edition (2000), content of which is herein incorporated by reference. Latent tuberculosis treatment usually uses a single antibiotic, while active tuberculosis disease is treated with combinations of several antibiotics, to reduce the risk of the bacteria developing antibiotic resistance (O'Brien, R. “Drug-resistant tuberculosis: etiology, management and prevention”. Semin Respir Infect 9 (2): 104-112 (1994), content of which is herein incorporated by reference). Primary resistance occurs in subjects who are infected with a resistant strain of tuberculosis. A subject with fully susceptible tuberculosis can develop secondary resistance (acquired resistance) during tuberculosis therapy because of inadequate treatment or not taking the prescribed regimen appropriately. Accordingly, a subject can be one who is unresponsive to treatment with one or more antibiotics.

Without wishing to be bound by a theory, the compounds of the invention are useful for treatment of multi-drug-resistant tuberculosis (MDR-TB) and/or extensively drug-resistant tuberculosis (XDR-TB) because they represent novel structural class of compounds. As used herein, the term “multi-drug-resistant tuberculosis” refers to tuberculosis that is resistant to the two most effective first-line TB drugs: rifampicin and isoniazid. As used herein, “extensively drug-resistant tuberculosis” refers to tuberculosis that is also resistant to three or more of the six classes of second-line drugs. See for example, Centers for Disease Control and Prevention (CDC), “Emergence of Mycobacterium tuberculosis with extensive resistance to second-line drugs—worldwide, 2000-2004”. MMWR Morb Mortal Wkly Rep 55 (11): 301-3055 (2006), content of which is herein incorporated by reference.

### EXAMPLES

The invention now being generally described, it will be more readily understood by reference to the following examples which are included merely for purposes of illustration of certain aspects and embodiments of the present invention, and are not intended to limit the invention.

The compounds of the inventions may be prepared by any process known to be applicable to the preparation of chemically-related compounds. Necessary starting materials may be obtained by standard procedures of organic chemistry. Alternatively necessary starting materials are obtainable by analogous procedures to those illustrated which are within the ordinary skill of a chemist. The compounds and processes of the present invention will be better understood in connection with the following representative synthetic schemes and examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

### General Experimental Methods

Melting points were determined on a capillary melting point apparatus and are uncorrected. All ¹H NMR spectra were recorded at 93.94 kG (¹H 400 MHz), and ¹³C NMR spectra were recorded at 70.5 kG (¹³C 75 MHz) or 94.04 kG (¹³C 100 MHz) at ambient temperature in CDCl₃, CD₃OD, or CD₃CN as indicated. Hydrogen chemical shifts are expressed in parts per million (ppm) relative to the residual protio solvent resonance in CDCl₃ (δ 7.24 for residual CHCl₃), CD₃OD (δ 3.31 for residual CHD₂OD) or CD₃CN (δ 1.94 for residual CHD₂CN). For ¹³C spectra, the center line (δ 77.0) of the CDCl₃ triplet, the center line (δ 49.15) of the CD₃OD septet or the center line (δ 1.39) of the CD₃CN septet was used as the internal reference. Unless otherwise noted, each carbon resonance represents a single carbon (relative intensity). Infrared spectra were recorded on NaCl plates prepared by depositing a solution of the sample on the NaCl plate in an appropriate, volatile solvent (typically CHCl₃) followed by evaporation of the solvent. Only diagnostic bands (OH, NH and CN stretching frequencies) are reported. High resolution mass spectra (HRMS) was obtained using electron impact (EIMS, 70 eV) or chemical ionization (CIMS, 140 eV) mode of ionization on a double focusing mass spectrometer, or on a quadrupolar time-of-flight (Q-TOF) mass spectrometer in either LC-electrospray (ESI-LC/MS) or atmospheric pressure chemical ionization (APCI) positive ion mode, as noted. For ESI-LC/MS, a 10-90% gradient CH₃CN (aqueous) solvent system was employed, 0.5 mL/min flow rate on a C₁₈ column (5 μm, 4.6 i.d.×50 mm) with nitrogen nebulizer gas, source temperature of 150° C., desolvation temperature of 250° C., capillary voltage of 1.6 kV, cone voltage of 38-48 V (ramp). Flash chromatography was performed on silica gel-60 (43-60 μm) (Still, et al., J. Org. Chem. 43, 2923-2925 (1978)). Optical rotation ([α]²⁵_{D}) concentrations “c” are given in g/100 mL. Library synthesis was carried out with MiniBlock and MiniBlock XT parallel synthesis systems. The following solvents were freshly distilled immediately prior to use: THF distilled from sodium/benzophenone, methanol distilled from magnesium/iodide, and CH₂Cl₂ distilled from calcium hydride. Other commercially available starting materials and anhydrous solvents (DMF, dichloroethane, chlorobenzene) were used without further purification. The CpCo(CO)₂ catalyst, epoxides, propargyl bromide, all alkynes, acyl chlorides, isocyanates and sulfonyl chlorides were commercially available.

### Example 1

### Design and Synthesis of Tetrahydronaphthyridine Scaffolds and the Compound Library

Small, unnatural heterocycles, such as tetrahydronaphthyridines, are of interest as library scaffolds. See for example, Lahue, et al., J. Org. Chem. 69, 7171-7182 (2004) and Woo, et al., Tetrahedron, 63, 5649-5655 (2006), content of both of which is herein incorporated by reference. To this end, a microwave-promoted, cobalt-catalyzed [2+2+2] cyclization (Scheme 1) has been recently reported to prepare pyrano-annulated 5,6,7,8-tetrahydro-1,6-naphthyridines (1, n=2), 6,7-dihydro-5H-pyrrolo[3,4-b]pyridines (1, n=1) and 6,7,8,9-tetrahydro-5H-pyrido[2,3-d]azepines (1, n=3). See Zhou, et al., Org. Lett. 9, 393-396 (2007), content of which is herein incorporated by reference in its entirety. With multiple sites for further functionalization (R, R′, R″, and “n”) and low molecular weight, these compounds have several features desirable in library scaffolds. In contrast to the fully aromatized naphthyridines which have shown significant bioactivities, tetrahydronaphthyridines have not received significant attention. See for example, Chan, et al., J. Med. Chem. 42, 3023-3025 (1999); Chan, et al., Bioorg. Med. Chem. Lett. 9, 2583-2586 (1999); Chan, Med. Chem. Lett. 11, 103-105 (2001); Shiozawa, et al., Chem. Pharm. Bull. 32, 995-1005 (1984); Shiozawa, et al., Chem. Pharm. Bull. 32, 2522-2529 (1984); Shiozawa, et al., Chem. Pharm. Bull. 32, 3981-3993 (1984); and Shiozawa, et al., Chem. Pharm. Bull. 33, 5332-5340 (1985), content of all of which is herein incorporated by reference.

To probe the biological effects of this class of heterocycles, four tetrahydropyranonaphthyridines (Scheme 2, 1{1-4}) with different substitutions at the C6 and C8 positions were chosen as scaffolds for the preparation of a first generation library. The secondary amine at N2 was then utilized as the functionalization point for formation of ureas, amides and sulfonamides.

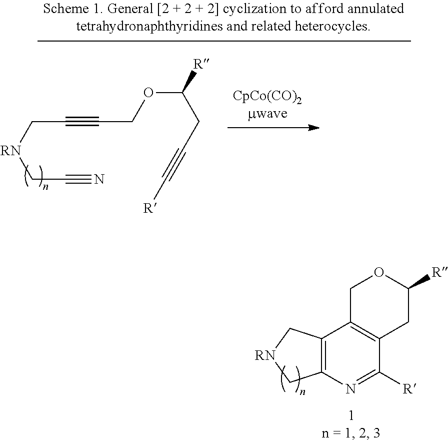

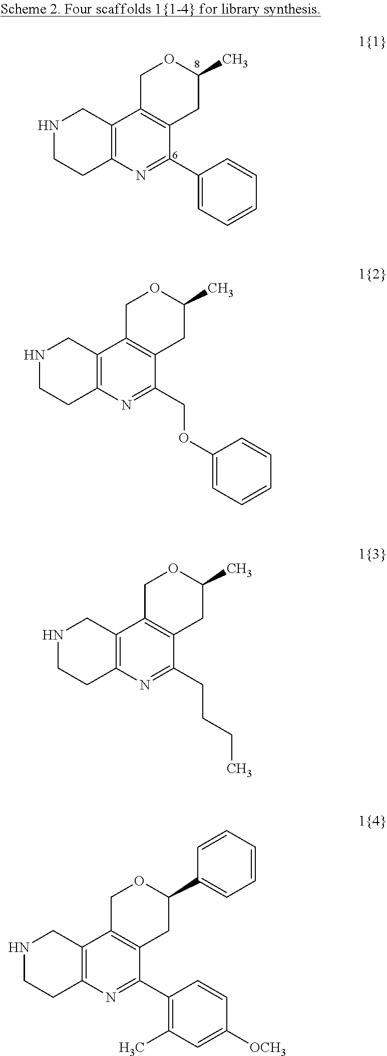

### Synthesis of Tetrahydronaphthyridine Scaffolds 1{1-4}

The synthetic protocol of scaffolds 1{1-4} is shown as in Scheme 3. See Zhou, et al. Org. Lett. 9, 393-396 (2007). The synthesis began with the preparation of four dialkynyl ethers 3a-3d (Scheme 3). (S)-propylene oxide and (S)-styrene oxide were opened with the appropriate lithium acetylides under Lewis acid activation to afford the secondary homopropargyl alcohols 2a-2d. See for example, Yamaguchi, et al., M.; Nobayashi, Y.; Hirao, I. Tetrahedron, 40, 4261-4266 (1984) and Shindo, et al., Tetrahedron Lett. 45, 9265-9268 (2004), content of both of which is herein incorporated by reference. The preparation of alcohols 2a-c utilized BF3.OEt₂ at −78° C., while 2d was synthesized at 0° C. with LiClO₄ as the catalyst. Propargylations of alcohols 2a-2d produced dialkynyl ethers 3a-3d. See for example, Brondel, et al., Tetrahedron Lett. 47, 9305-9308 (2006), content of which is herein incorporated by reference. Copper promoted Mannich reactions using p-methoxybenzyl-protected aminonitrile 4 gave dialkynyl aminonitriles 5a-5d. The p-methoxybenzyl-protected aminonitrile 4 is easily prepared by conjugate addition of p-methoxybenzylamine to acrylonitrile as described, for example, in Moloney, et al., Molecules, 6, 230-243 (2001); Hernandez-Rodriguez, et al., J. Phys. Org. Chem. 18, 792-799 (2005); and Bew, et al., J. Chem. Soc., Chem. Commun. 4338-4340 (2006), content of all of which is herein incorporated by reference. Copper promoted Mannich reactions are described, for example in, Su, et al., Tetrahedron, 60, 8645-8657 (2004) and Kabalka, et al, Tetrahedron, 62, 857-867 (2006), content of both of which is herein incorporated by reference. The [2+2+2] cyclizations of 5a-5d catalyzed by CpCo(CO)₂ proceeded readily under microwave irradiation as previously reported by Zhou et al. (2007), giving p-methoxybenzyl-protected 5,6,7,8-tetrahydro-1,6-naphthyridines 6a-6d in good yields. Deprotection of the PMB group by Pd-catalyzed hydrogenation afforded the scaffolds 1{1-4}. Deprotection of the PMB group by Pd-catalyzed hydrogenation is described in Trost, et al., J. Am. Chem. Soc. 118, 6297-6298 (1996), content of which is herein incorporated by reference.

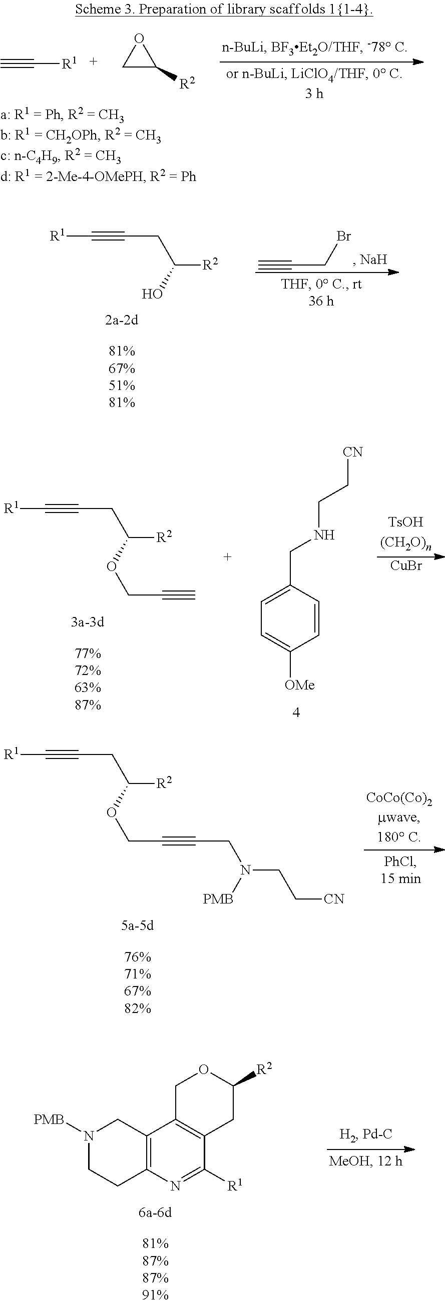

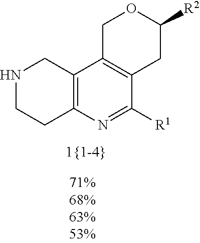

### Design of the Compound Library.

Further functionalization of the scaffolds 1 were accomplished through three main reactions (Scheme 4): (1) urea formation with isocyanates, (2) amide formation with acyl chlorides, and (3) sulfonamide formation with sulfonyl chlorides. These reactions are described, for example, in DeVries, et al., J. Med. Chem. 32, 2318-2325 (1989); Esler, et al., Bioorg. Med. Chem. Lett. 14, 1935-1938 (2004); Huang, et al., Tetrahedron Lett. 28; 547-550 (1987); Jenkins, et al., J. Org. Chem. 69, 8565-8573 (2004); Nie, et al., Bioorg. Med. Chem. Lett. 16, 5513-5516 (2006); and Becker, et al., J. Med. Chem. 49, 3116-3135 (2006), content of all of which is herein incorporated by reference. Numerous reagents (for example, reagents of isocyanates, acyl chlorides and sulfonyl chlorides) and subsequent products were submitted to diversity analysis, and the most dissimilar set of reagents were selected as library members for further functionalization of the main scaffolds. See for example, Mason, et al., J. Mol. Gr. Mod. 18, 438-451 (2000); Tounge, et al., J. Chem. Inf. Comput. Sci. 42, 879-884 (2002); Blake, J. F. Curr. Opin. Chem. Biol. 2004, 8, 407-411 (2004); and Savchuk, et al., Curr. Opin. Chem. Biol. 8, 412-417 (2004), content of all of which is herein incorporated by reference. Eight reagents were then selected for each method of functionalization and for each of the scaffolds 1{1-4}, resulting a 96-member compound library. Reductive aminations were also successful, however the resulting tertiary amines were not as stable as products prepared by other methods of functionalization for long-term storage. Five such tertiary amines were prepared. Thus, a total of 101-member compound library were synthesized using the tetrahydronaphthyridine scaffolds 1{1-4}.

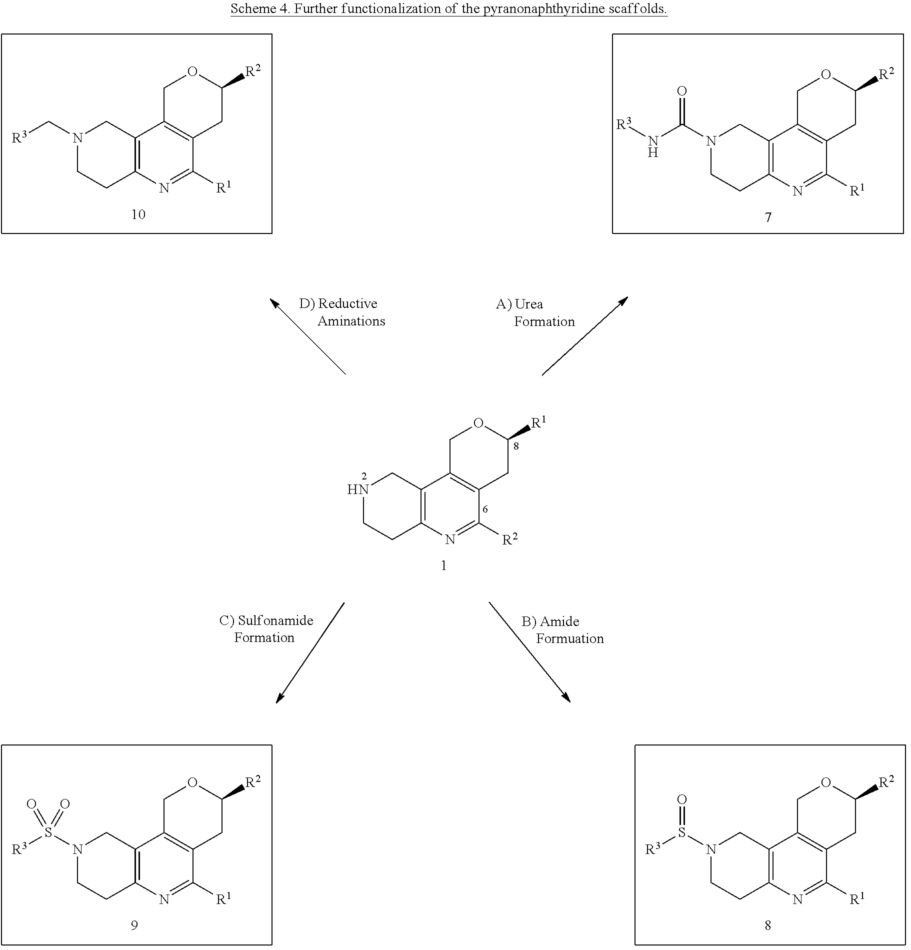

### Synthesis of the Compound Library.

a. Functionalization of the Scaffolds by Urea Formation

The reaction conditions for urea formation were optimized with the reaction between scaffold 1{1} and three isocyanates. The reactions were carried out using 1.1 equivalent of isocyanates in dichloroethane (65° C., 4 h), followed by treatment with PS-trisamine resin (1.1 equiv) at room temperature for 6 hours to scavenge excess isocyanate. The PS-trisamine resin is described in Booth, R. J. and Hodges, J. C. J. Am. Chem. Soc. 1997, 119, 4882-4886, content of which is herein incorporated by reference. In most cases, LC/MS indicated 90-95% conversions from scaffold 1{1} to urea products upon filtration as determined by ELS, and NMR spectra of the crude reaction residues showed only desired urea products were obtained. The isolated yields of ureas were 91% (7{1,1}), 75% (7{1,4}) and 83% (7{1,5}) following flash chromatography (see Table 1).

Using the optimized reaction conditions, a 32-member compound sublibrary was prepared from the four tetrahydronaphthyridine scaffolds 1{1-4} and eight commercially available isocyanates selected by the diversity analysis. The crude products were purified by mass-directed preparative HPLC to provide ureas 7{1-4, 1-8} in the yield of <10 to >98%, with >90% purity of the library members (Table 1).

b. Functionalization of the Scaffolds by Amide Formation

Synthesis of amides started with model studies using scaffold 1{1} and acyl chlorides (1.1 equiv). The reactions proceeded readily with triethylamine as base with excess acid chloride. While full conversions of scaffold 1{1} to amide products were observed by NMR, unscavenged acyl chloride was also present. PS-DMAP (1.5 equiv) was utilized both as a catalyst and an acid chloride scavenger as described by Shai, et al., J. Am. Chem. Soc. 1985, 107, 4249-4252 (1985), content of which is herein incorporated by reference). The reactions were carried out at room temperature in dichloromethane for 10 hours. The reactions proceeded to completion, and upon filtration, NMR spectra indicated full conversion to the expected products with no acid chlorides detected. Following flash chromatography, the isolated yields of amides were 93% (8{1,1}), 75% (8{1,2}), 81% (8{1,4}) and 78% (8{1,7}).

Using the optimized reaction conditions, a 32-member compound sublibrary was then prepared from the four tetrahydronaphthyridine scaffolds 1{1-4} and eight commercially available acid chlorides selected by the diversity analysis. To guarantee full conversion for each reaction, a slightly higher excess of acid chlorides (1.3 equiv) and PS-DMAP resin (1.7 equiv) were used for the library synthesis. After the reactions, the PS-DMAP resin was removed by filtration, and the solvent was evaporated to afford the crude library products. Purification by mass-directed preparative HPLC provided the library members 8{1-4, 1-8} in the yield of <10 to >98%, with >90% purity of library member (Table 2).

c. Functionalization of the Scaffolds by Sulfonamide Formation

The reaction conditions for sulfonamide formation were performed with the reaction between scaffold 1{1} and two sulfonyl chlorides (Table 3). Under optimal conditions, equal molar equivalents of 1{1} and sulfonyl chlorides were allowed to react at room temperature for 4 hours in the presence of triethylamine (TEA, 1 equiv). No starting material was observed by LC/MS, and the isolated yields of the desired sulfonamides after chromatography were 93% (9{1,1}) and 81% (9{1,3}).

Using these reaction conditions, a 32-member sulfonamide sublibrary was then prepared from the four tetrahydronaphthyridine scaffolds 1{1-4} and eight commercially available sulfonyl chlorides selected by the diversity analysis. A longer reaction time (10 h) and higher temperature (55° C.) in comparison to the model study were used to enhance the conversion rate. After evaporating the solvent, the crude reaction mixtures were purified by mass-directed preparative HPLC to afford the library members 9{1-4, 1-8} in the yield of <10 to >85%, with >90% purity of library member (Table 3).

d. Functionalization of the Scaffolds by Reductive Aminations

The reductive amination reactions were carried out between naphthyridine scaffolds 1{1-4} and five different aldehydes (Table 4). See for example, Brinner, et al., Bioorg. Med. Chem. 10, 3649-3661 (2002). In all cases, the aminations proceed to the desired tertiary amines 10a-10e with a good to excellent yields (78% to 88%). These amines were not observed to be as stable as products prepared by other methods of functionalization, and may slowly oxidize upon prolonged storage.

Using the cobalt-catalyzed [2+2+2] cyclization methodology, an efficient preparation of 5,6,7,8-tetrahydro-1,6-naphthyridine scaffolds was accomplished. Secondary amine functionalities at the N2 position, i.e., ureas, amides and sulfonamide formation, were accomplished on the four scaffolds through solution-phase parallel synthesis protocols and used to prepare compounds in the library. Easy workup and LC/MS purification provided high purity library products.

TABLE 1
Urea Sublibrary Members^{a}

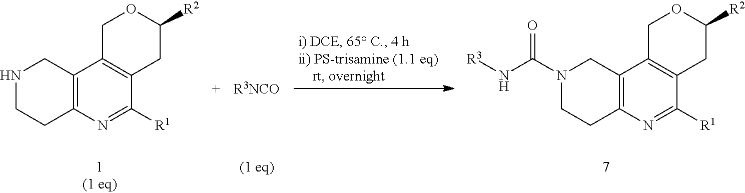

| Library Scaffolds Isocyanates | 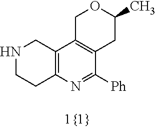 | 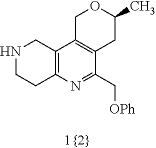 | 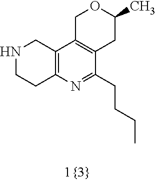 | 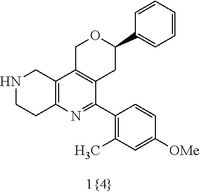 |
|---|---|---|---|---|
| 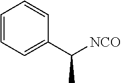 | 7{1,1} 91% (>98%) | 7{2,1} (91%) | 7{3,1} (>98%) | 7{4,1} (>98%) |
| 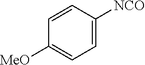 | 7{1,2} 78% | 7{2,2} (>98%) | 7{3,2} (>98%) | 7{4,2} (67%) |
| 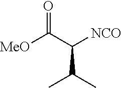 | 7{1,3} (>98%) | 7{2,3} (>98%) | 7{3,3} (>98%) | 7{4,3} (>98%) |
| 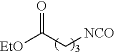 | 7{1,4} 75% (>98%) | 7{2,4} (>98%) | 7{3,4} (73%) | 7{4,4} (>98%) |
| 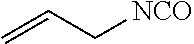 | 7{1,5} 83% (>98%) | 7{2,5} (>98%) | 7{3,5} (82%) | 7{4,5} (>98%) |
| 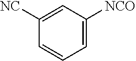 | 7{1,6} (92%) | 7{2,6} (>98%) | 7{3,6} (>98%) | 7{4,6} (>98%) |
| 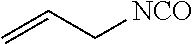 | 7{1,7} (>98%) | 7{2,7} (>98%) | 7{3,7} (>98%) | 7{4,7} (>98%) |
| 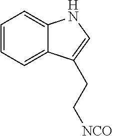 | 7{1,8} (<10%) | 7{2,8} (<10%) | 7{3,8} (<10%) | 7{4,8} (>98%) |

^{a}Yields without parentheses are isolated yields with library protocols for individual compounds. Yields within parentheses are LC-MS yields from library
preparation.

TABLE 2
Amide Sublibrary Members^{a}

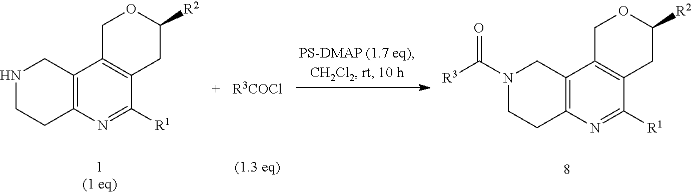

| Library Scaffolds Acid Chlorides | 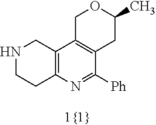 | 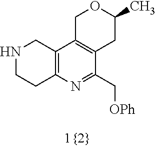 | 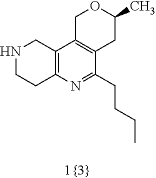 | 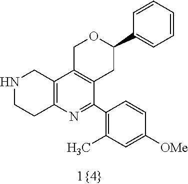 |
|---|---|---|---|---|
| 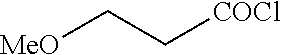 | 8{1,1} 93% (85%) | 8{2,1} (91%) | 8{3,1} (>98%) | 8{4,1} (>98%) |
| 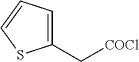 | 8{1,2} 75% (<10%) | 8{2,2} (>98%) | 8{3,2} (68%) | 8{4,2} (>98%) |
| 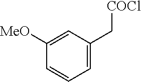 | 8{1,3} (61%) | 8{2,3} (>98%) | 8{3,3} (78%) | 8{4,3} (>98%) |
| 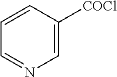 | 8{1,4} 81% (73%) | 8{2,4} (>98%) | 8{3,4} (83%) | 8{4,4} (74%) |
| 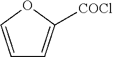 | 8{1,5} (67%) | 8{2,5} (>98%) | 8{3,5} (75%) | 8{4,5} (76%) |
| 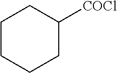 | 8{1,6} (68%) | 8{2,6} (89%) | 8{3,6} (38%) | 8{4,6} (45%) |
| 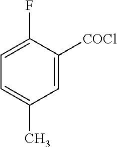 | 8{1,7} 78% (27%) | 8{2,7} (64%) | 8{3,7} (15%) | 8{4,7} (86%) |
| 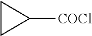 | 8{1,8} (66%) | 8{2,8} (72%) | 8{3,8} (>98%) | 8{4,8} (>98%) |

^{a}Yields without parentheses are isolated yields applying the library procedure for individual compounds. Yields within parentheses are LC-MS yields from
library preparation.

TABLE 3
Sulfonamide Sublibrary Members^{a}

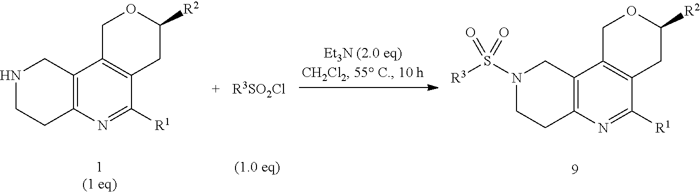

| Library Scaffolds Sulfonyl Chlorides | 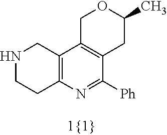 | 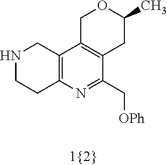 | 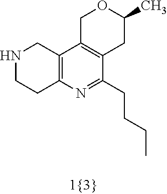 | 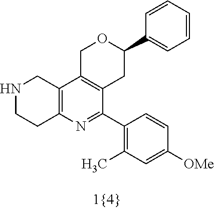 |
|---|---|---|---|---|
| 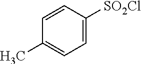 | 9{1,1} 93% (33%) | 9{2,1} (95%) | 9{3,1} (10%) | 9{4,1} (15%) |
| 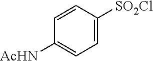 | 9{1,2} (16%) | 9{2,2} (76%) | 9{3,2} (60%) | 9{4,2} (93%) |
| 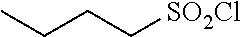 | 9{1,3} 81% (43%) | 9{2,3} (74%) | 9{3,3} (65%) | 9{4,3} (76%) |
| 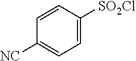 | 9{1,4} (24%) | 9{2,4} (40%) | 9{3,4} (97%) | 9{4,4} (28%) |
| 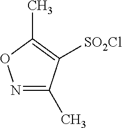 | 9{1,5} (50%) | 9{2,5} (55%) | 9{3,5} (52%) | 9{4,5} (6%) |
| 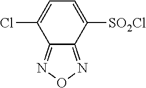 | 9{1,6} (5%) | 9{2,6} (92%) | 9{3,6} (12%) | 9{4,6} (60%) |
| 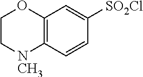 | 9{1,7} (98%) | 9{2,7} (68%) | 9{3,7} (27%) | 9{4,7} (15%) |
| 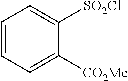 | 9{1,8} (86%) | 9{2,8} (73%) | 9{3,8} (41%) | 9{4,8} (64%) |

^{a}Yields without parentheses are isolated yields applying the library procedure for individual compounds. Yields in parentheses are LC-MS yields from library preparation.

TABLE 4
Reductive aminations with the scaffolds 1{1-4}^{a}

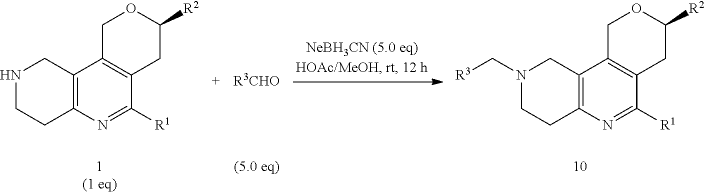

| Entry | R¹ | R² | R³ | Yield (10)^{a} |
|---|---|---|---|---|
| 1 | Ph | Me | 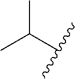 | 82% (10a) |
| 2 | Ph | Me | 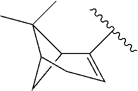 | 78% (10b) |
| 3 | CH₂OPh | Me | 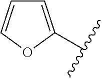 | 80% (10c) |
| 4 | n-Bu | Me | 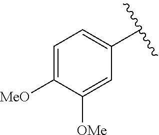 | 88% (10d) |
| 5 | 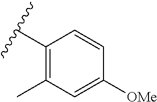 | Ph | 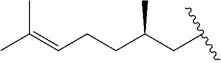 | 88% (10e) |

^{a}Isolated yields.

### Example 2

### Procedure A

### Epoxide Opening for the Preparation of 2a-2d

(S)-5-Phenylpent-4-yn-2-ol (2a): To a stirred solution of 1-ethynylbenzene (1.0 mL, 9.10 mmol) in THF (40 mL) at −78° C., n-BuLi (1.6 M in hexanes, 6.3 mL, 1.1 equiv) was added dropwise. After stirring 1 h at −78° C., BF₃.OEt₂ (1.4 mL, 1.2 equiv) was added dropwise and the stirring continued for an additional 15 min. (S)-2-Methyloxirane (0.95 mL, 13.70 mmol, 1.5 equiv) in anhydrous THF (30 mL) was then added dropwise at −78° C. Stirring was continued for 3 h at −78° C., then the reaction was quenched with saturated NH₄Cl solution (120 mL). The mixture was extracted with ether (3×150 mL), and the combined organic layers were washed with saturated brine (150 mL), dried over sodium sulfate, and the solvent removed in vacuo. The residues were purified by flash chromatography (hexanes/EtOAc, 4:1, R_{f}, 0.25) on silica gel to afford the secondary alcohol 1a as a white solid (1.179 g, 81% yield): mp 62-64° C.; [α]²⁵_{D} +13.9 (c=0.7, CHCl₃); IR (NaCl) 3342 cm⁻¹; ¹H NMR (400 MHz, CDCl₃) δ 7.42-7.44 (m, 2H), 7.29-7.32 (overlapped, 3H), 4.07 (ddq, J=6.8, 5.2, 6.0 Hz, 1H), 2.64 (dd, J_{AB}=16.5, J=5.2 Hz, 1H), 2.57 (dd, J_{AB}=16.5 Hz, J=6.8 Hz, 1H), 1.85 (br s, OH), 1.34 (d, J=6.0 Hz, 3H); ¹³C NMR (75 MHz, CDCl₃) δ 131.6 (2C), 127.8, 128.2 (2C), 123.4, 86.5, 82.7, 66.4, 29.8, 22.3; CIMS (NH₃), m/z (%) 160 ([M]⁺, 1), 131 (30), 115 (33), 83 (100), 69 (55); HRMS calcd for C₁₁H₁₃O 161.0966 (APCI) m/z 161.0963 (M+H).

(R)-4-(4-Methoxy-2-methylphenyl)-1-phenylbut-3-yn-1-ol (2d) (Shindo et al., 2004): To a stirred THF (10 mL) solution of 1-ethynyl-4-methoxy-2-methylbenzene (0.590 g, 4.04 mmol), at 0° C. was added dropwise n-BuLi (1.6 M in hexanes, 2.5 mL, 4.0 mmol). After stirring 10 min at 0° C., a solution of (S)-2-phenyloxirane (0.23 mL, 2.0 mmol) and LiClO₄ (0.430 g, 4.05 mmol) in THF (3.0 mL) was added dropwise. The reaction was allowed to warm to room temperature and the stirring continued for another 12 h, then the reaction was quenched with saturated NH₄Cl solution (15 mL). The mixture was extracted with ether (3×40 mL), then the combined organic layers were washed with saturated brine (150 mL), dried over sodium sulfate, then the solvent removed in vacuo. The residues were purified by flash chromatography (hexanes:EtOAc, 4:1, R_{f} 0.53) to afford 2d as light yellow oil (417.6 mg, 1.56 mmol, 78% yield): [α]²⁵_{D} +26.0 (c 0.92, CHCl₃); IR (NaCl) 3400, 2805, 1605, 1479, 1235, 1050, 700 cm⁻¹; ¹H NMR (400 MHz, CDCl₃) δ 7.42 (br d, J=7.0 Hz, 2H), 7.35 (br dd, J=8.4, 7.0 Hz, 2H), 7.28 (tt, J=8.4, 1.4 Hz, 1H), 7.26 (d, J=8.4 Hz, 1H), 6.68 (d, J=2.7 Hz, 1H), 6.63 (dd, J=8.4, 2.7 Hz, 1H), 4.92 (br dd, J=7.0, 5.9 Hz, 1H), 3.75 (s, 3H), 2.89 (dd, J_{AB}=16.8, J_{AX}=5.9 Hz, 1H), 2.87 (dd, J_{AB}=16.8, J_{BX}=7.0 Hz, 1H), 2.43 (br s, OH), 2.29 (s, 3H); ¹³C NMR (75 MHz, CDCl₃) δ 159.4, 142.9, 142.1, 133.4, 128.6 (2C), 128.1, 126.1 (2C), 115.5, 115.2, 111.3, 88.1, 82.2, 72.9, 55.4, 30.9, 21.2; LCMS (ESI), m/z (%) 267 ([M+1]⁺, 100); HRLCMS (ESI) m/z 267.1371 ([M+1]⁺, 100%) calcd for C₁₈H₁₉O₂ 267.1385.

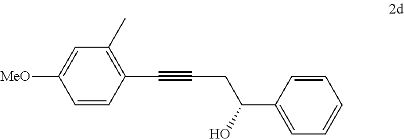

### Example 3

### Procedure B

### Propargylation of Chiral Secondary Homopropargyl Alcohols (2→3, 3a-3d)

(S)-4-(Prop-2-ynyloxy)pent-1-ynylbenzene (3a): A solution of (S)-5-phenylpent-4-yn-2-ol (2a, 0.237 g, 1.48 mmol) in THF (10 mL) was added dropwise into a suspension of sodium hydride (40.0 mg, 1.1 equiv) in THF (5 mL) at 0° C. After stirring for 1 h at 0° C., a solution of propargyl bromide (0.529 g, 4.44 mmol, 3.0 equiv) in THF (5 mL) was added dropwise. The solution was allowed to warm to room temperature and stirring continued for 36 h. The reaction was quenched with water (30 mL) and the reaction mixture was extracted with ether (3×50 mL). The combined organic layers were washed with saturated brine (80 mL), dried over sodium sulfate, and then the solvent removed in vacuo. The residues were purified by flash chromatography (hexanes/EtOAc, 4:1, R_{f}, 0.73) on silica gel to afford the propargyl ether 3a as a light yellow oil (0.226 g, 77% yield): [α]²⁵_{D} −21.3 (c 2.2, CHCl₃); IR (NaCl) 3292, 2119, 758, 692 cm⁻¹; ¹H NMR (400 MHz, CDCl₃) δ 7.37-7.40 (m, 2H), 7.24-7.28 (overlapped, 3H), 4.24 (ABX, J_{AB}=15.6, J_{AX}=J_{BX}=2.2 Hz, 2H), 3.90 (ddq, J=7.0, 4.8, 6.4 Hz, 1H), 2.70 (dd, J_{AB}=16.5, J_{BX}=4.8 Hz, 1H), 2.53 (dd, J_{AB}=16.5, J_{AX}=7.0 Hz, 1H), 2.42 (t, J=2.2 Hz, 1H), 1.33 (d, J=6.4 Hz, 3H); ¹³C NMR (75 MHz, CDCl₃) δ 131.3 (2C), 128.0 (2C), 127.6, 123.4, 86.2, 82.0, 79.7, 74.0, 73.0, 55.8, 26.6, 19.3; CIMS (NH₃), m/z (%) 199 ([M+1]⁺, 43), 198 ([M]⁺, 36), 197 (28), 183 (22), 155 (27), 154 (77), 143 (60), 128 (28), 115 (64), 105 (67), 83 (100); HRMS (CI, NH₃) m/z 198.1046 ([M]⁺, 36%), calcd for C₁₄H₁₄O 198.1045.

(R)-4-Methoxy-2-methyl-1-(4-phenyl-4-(prop-2-ynyloxy)but-1-ynyl)benzene (3d): A solution of (R)-4-(4-methoxy-2-methylphenyl)-1-phenylbut-3-yn-1-ol (2a, 0.532 g, 2.0 mmol) in THF (13 mL) was added dropwise into a suspension of sodium hydride (60% suspension in mineral oil, 54.0 mg, 2.2 mmol) in THF (6.5 mL) at 0° C. with stirring. After stirring for 1 h at 0° C., a solution of propargyl bromide (0.714 g, 6.0 mmol) in THF (6.5 mL) was added dropwise. The solution was allowed to warm to room temperature and stirring continued for 48 h. The reaction was quenched with water (30 mL) and the reaction mixture was extracted with ether (3×50 mL). The combined organic layers were washed with saturated brine (80 mL), dried over sodium sulfate, and the solvent removed in vacuo. The residues were purified by flash chromatography (hexanes/EtOAc, 4:1, R_{f} 0.91) to afford 3d as a light yellow oil (529.0 mg, 1.74 mmol, 87% yield): [α]²⁵_{D} +40.3 (c 2.68, CHCl₃); IR (NaCl) 3288, 2909, 1605, 1498, 1236, 1087, 701 cm⁻¹; ¹H NMR (400 MHz, CDCl₃) δ 7.38 (br dd, J=7.8, 7.0 Hz, 2H), 7.33 (br d, J=7.8 Hz, 2H), 7.29 (tt, J=7.0, 1.6 Hz, 1H), 7.19 (d, J=8.4 Hz, 1H), 6.65 (d, J=2.5 Hz, 1H), 6.59 (dd, J=8.4, 2.5 Hz, 1H), 4.73 (dd, J=7.0, 6.4 Hz, 1H), 4.16 (dd, J_{AB}=15.7, J_{AX}=2.2 Hz, 1H), 3.93 (dd, J_{AB}=15.7, J_{BX}=2.6 Hz, 1H), 3.74 (s, 3H), 2.98 (dd, J_{AB}=16.8, J_{AX}=6.4 Hz, 1H), 2.83 (dd, J_{AB}=16.8, J_{BX}=7.0 Hz, 1H), 2.39 (dd, J=2.6, 2.2 Hz, 1H), 2.21 (s, 3H); ¹³C NMR (75 MHz, CDCl₃) δ 159.2, 142.0, 139.9, 133.2, 128.6 (2C), 128.4, 127.3 (2C), 115.8, 115.0, 111.1, 88.3, 81.3, 79.8, 79.4, 74.6, 56.1, 55.3, 28.9, 21.0; HRLCMS (ESI) m/z 305.1536 ([M+1]⁺, 100%) calcd for C₂₁H₂₁O₂ 305.1542.

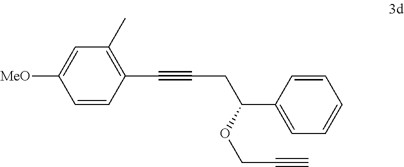

### Example 5

### Procedure C

### Alkynyl Mannich Reactions (3+4→5, 5a-5d)

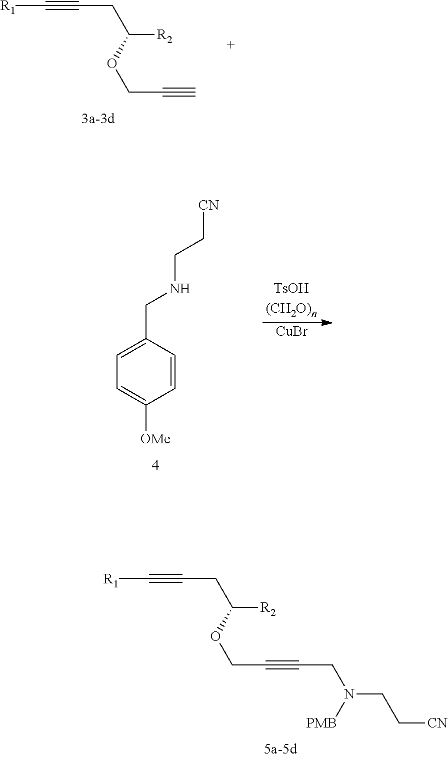

(S)-34(4-Methoxybenzyl)(4-(5-phenylpent-4-yn-2-yloxy)but-2-ynyl)amino)propanenitrile (5a): A solution of 3-(4-methoxybenzylamino)propanenitrile (4, 96.0 mg, 0.5 mmol, 1.0 equiv), paraformaldehyde (61.0 mg) and p-toluenesulfonic acid (1.0 equiv, 96.0 mg) in dichloromethane (2 mL) was sealed in a 10 mL microwave reaction vessel (CEM Corporation) and purged with nitrogen. The reaction was heated to 60° C. with stirring for 8 h, then the solvent was removed in vacuo to afford a crude residue. (S)-(4-(Prop-2-ynyloxy)pent-1-ynyl)benzene (3a, 100.0 mg, 1.0 equiv) dissolved in THF/DMF (2:1, 3 mL), was then added into the resulting crude residue, followed by the addition of CuBr (36.0 mg, 0.5 equiv). The mixture was heated with stirring to 70° C. for 48 h. The reaction was quenched with water (10 mL) and the mixture was extracted with EtOAc (3×10 mL). The combined organic layers were washed with saturated brine, dried over sodium sulfate, then the solvent was removed in vacuo. The residue was purified by flash chromatography (hexanes/EtOAc, 3:1, R_{f}0.30) on silica gel to yield dialkynyl nitrile 5a as a yellow oil (153.2 mg, 76% yield): [α]²⁵_{D} −11.3 (c 3.46, CHCl₃); IR (NaCl) 2931, 1612, 1512, 1247, 1104, 1035, 758, 693 cm⁻¹; ¹H NMR (400 MHz, CDCl₃) δ 7.36 (m; 2H), 7.25-7.21 (overlap, 5H), 6.80 (d, J=8.4 Hz, 2H), 4.28 (ABX₂, J_{AB}=15.8 Hz, J_{AX}=J_{AX′}=1.8 Hz, 1H), 4.27 (ABX₂, J_{AB}=15.8 Hz, J_{BX}=J_{BX′}=1.8 Hz, 1H), 3.88 (ddq, J=7.1, 4.9, 6.2 Hz, 1H), 3.74 (s, 3H), 3.57 (s, 2H), 3.36 (br s, 2H), 2.81 (t, J=5.8 Hz, 2H), 2.70 (dd, J_{AB}=16.6, J_{AX}=4.9 Hz, 1H), 2.53 (dd, J_{AB}=16.6, J_{BX}=7.1 Hz, 1H), 2.41 (t, J=5.8 Hz, 2H), 1.32 (d, J=6.2 Hz, 3H); ¹³C NMR (75 MHz, CDCl₃) δ 158.9, 131.5 (2C), 130.0 (2C), 129.7, 128.2 (2C), 127.7, 123.5, 118.6, 113.7 (2C), 86.5, 82.2, 81.9, 80.0, 73.0, 57.1, 56.2, 55.1, 48.4, 41.5, 26.8, 19.5, 16.6; HRLCMS (ESI) m/z 401.2235 ([M+1]⁺, 15%) calcd for C₂₆H₂₉N₂O₂ 401.2229.

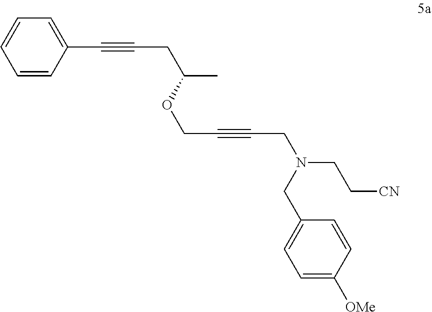

(S)-3-((4-Methoxybenzyl)(4-(6-phenoxyhex-4-yn-2-yloxy)but-2-ynyl)amino)propanenitrile (5b): Compound 5b was prepared according to Procedure C, as described above, beginning with (S)-(5-(prop-2-ynyloxy)hex-2-ynyloxy)benzene (3b, 130.0 mg, 0.57 mmol), 3-(4-methoxybenzylamino)propanenitrile 4 (108.0 mg, 0.57 mmol) and paraformaldehyde (67.4 mg). Purification by flash chromatography (hexanes/EtOAc, 4:1, R_{f} 0.17) gave 5b as a yellow oil (176.0 mg, 0.41 mmol, 71% yield): [α]²⁵_{D} −9.9 (c 2.25, CHCl₃); IR (NaCl) 2927, 2853, 2353, 2247, 1600, 1503, 1241, 1111, 1029, 828, 757 cm⁻¹; ¹H NMR (400 MHz, CDCl₃) δ 7.28-7.22 (overlap, 4H), 6.95 (overlap, 1H), 6.94 (d, J=8.2 Hz, 2H), 6.83 (d, J=8.2 Hz, 2H), 4.65 (dd, J=2.2, 2.0 Hz, 2H), 4.21 (br s, 2H), 3.79-3.76 (overlap, 1H), 3.76 (s, 3H), 3.58 (br s, 2H), 3.36 (br s, 2H), 2.81 (t, J=6.8 Hz, 2H), 2.52 (ddt, J_{AB}=16.6, J=4.4, 2.0 Hz, 1H), 2.43 (t, J=6.8 Hz, 2H), 2.37 (ddt, J_{AB}=16.6, J=6.8, 2.2 Hz, 1H), 1.24 (d, J=6.2 Hz, 3H); ¹³C NMR (100 MHz, CDCl₃) δ 159.2, 158.0, 130.3 (2C), 129.9, 129.6 (2C), 121.5, 118.8, 115.1 (2C), 114.1 (2C), 84.5, 82.0, 80.3, 77.1, 73.0, 57.5, 56.52, 56.48, 55.5, 48.8, 42.0, 26.5, 19.6, 17.1; HRLCMS (ESI) m/z 431.2325 ([M+1]⁺, 75%) calcd for C₂₇H₃₁N₂O₃ 431.2335.

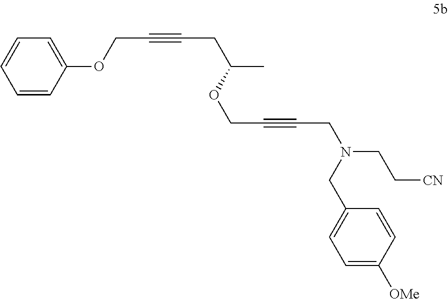

(S)-3-((4-Methoxybenzyl)(4-(non-4-yn-2-yloxy)but-2-ynyl)amino)propanenitrile (5c): Compound 5c was prepared according to Procedure C, as described above, beginning with (S)-2-(prop-2-ynyloxy)non-4-yne (3c, 100.0 mg, 0.56 mmol), 3-(4-methoxybenzylamino)propanenitrile 4 (107.0 mg, 0.56 mmol) and paraformaldehyde (67.3 mg). Purification by flash chromatography (hexanes/EtOAc, 4:1, R_{f} 0.17) gave 5c as a yellow oil (143.4 mg, 0.38 mmol, 67% yield): [α]²⁵₁₃-12.3 (c 2.21, CHCl₃); IR (NaCl) 2932, 2249, 1612, 1512, 1248, 1104, 1036, 824, 757 cm⁻; ¹H NMR (400 MHz, CDCl₃) δ 7.24 (d, J=8.6 Hz, 2H), 6.83 (d, J=8.6 Hz, 2H), 4.23 (br s, 2H), 3.77 (s, 3H), 3.74 (overlap, 1H), 3.59 (s, 2H), 3.37 (br s, 2H), 2.83 (t, J=7.0 Hz, 2H), 2.45 (t, J=7.0 Hz, 2H), 2.48-2.42 (overlap, 1H), 2.27 (dddd, J_{AB}=16.3, J=7.3, 2.4, 2.4 Hz, 1H), 2.13 (tt, J=7.0, 2.4 Hz, 2H), 1.48-1.31 (overlap, 4H), 1.25 (d, J=6.4 Hz, 3H), 0.87 (t, J=7.2 Hz, 3H); ¹³C NMR (75 MHz, CDCl₃) δ 159.1, 130.2 (2C), 129.8, 118.7, 113.9 (2C), 82.3, 82.2, 79.9, 76.3, 73.5, 57.4, 56.3, 55.3, 48.6, 41.8, 31.2, 26.2, 22.0, 19.5, 18.5, 16.9, 13.7; HRLCMS (ESI) m/z 381.2575 ([M+1]⁺, 8%) calcd for C₂₄H₃₃N₂O₂ 381.2542.

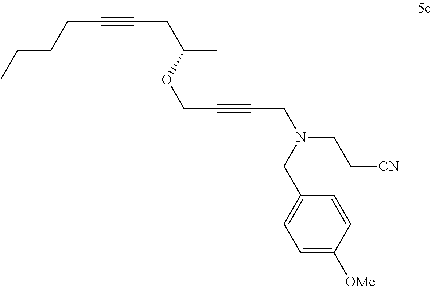

(R)-3-((4-(4-(4-Methoxy-2-methylphenyl)-1-phenylbut-3-ynyloxy)but-2-ynyl)(4-methoxybenzyl)amino)propanenitrile (5d): Compound 5d was prepared according to Procedure C, as described above, beginning with (R)-4-methoxy-2-methyl-1-(4-phenyl-4-(prop-2-ynyloxy)but-1-ynyl)benzene 3d (191.0 mg, 0.63 mmol), 3-(4-methoxybenzylamino)propanenitrile 4 (119.0 mg, 0.63 mmol) and paraformaldehyde (75.2 mg). Purification by flash chromatography (hexanes/EtOAc, 8:1, R_{f} 0.15) gave 5d as a clear oil (260.6 mg, 0.52 mmol, 82% yield): [α]²⁵_{D} +26.8 (c 2.90, CHCl₃); IR (NaCl) 2927, 2840, 2353, 1606, 1506, 1244, 1109, 1048, 823, 696 cm⁻¹; ¹H NMR (400 MHz, CDCl₃) δ 7.45-7.32 (overlap, 5H), 7.26 (d, J=8.6 Hz, 2H), 7.24 (d, J=8.4 Hz, 1H), 6.85 (d, J=8.6 Hz, 2H), 6.69 (d, J=2.6 Hz, 1H), 6.63 (dd, J=8.4, 2.6 Hz, 1H), 4.78 (dd, J=7.0, 6.6 Hz, 1H), 4.28 (ddd, J_{AB}=15.7, J=1.8, 1.7 Hz, 1H), 4.06 (ddd, J_{AB}=15.7, J=1.8, 1.6 Hz, 1H), 3.79 (s, 3H), 3.78 (s, 3H), 3.59 (s, 2H), 3.39 (br s, 2H), 3.02 (dd, J_{AB}=16.8, J_{AX}=6.6 Hz, 1H), 2.87 (dd, J_{AB}=16.8, J_{BX}=7.0 Hz, 1H), 2.81 (t, J=7.0 Hz, 2H), 2.43 (t, J=7.0 Hz, 2H), 2.27 (s, 3H); ¹³C NMR (75 MHz, CDCl₃) δ 159.2, 159.1, 141.9, 140.1, 133.1, 130.2 (2C), 129.8, 128.5 (2C), 128.3, 127.1 (2C), 118.7, 115.7, 115.0, 113.9 (2C), 111.1, 88.4, 81.7, 81.1, 80.5, 79.4, 57.3, 56.4, 55.3, 55.2, 48.6, 41.7, 29.0, 21.0, 16.9; HRLCMS (ESI) m/z 507.2675 ([M+1]⁺, 40%) calcd for C₃₃H₃₅N₂O₃ 507.2648.

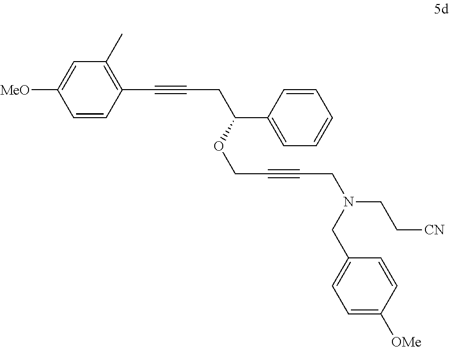

### Example 5

### Procedure D

### Microwave-Promoted Intramolecular Cobalt-Catalyzed [2+2+2] Cyclization (5→6, 6a-6d)

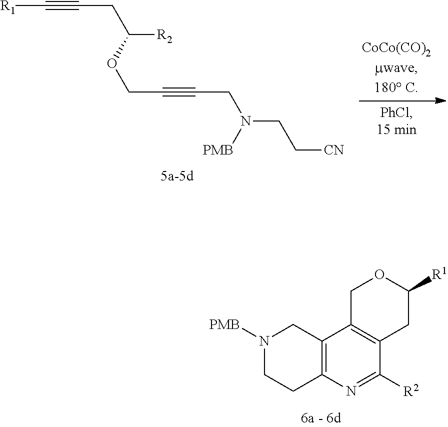

(S)-2-(4-Methoxybenzyl)-8-methyl-6-phenyl-2,3,4,7,8,10-hexahydro-1H-pyrano[4,3-c][1,6]naphthyridine (6a): A solution of dialkynylnitrile 5a (26.0 mg, 0.065 mmol) in chlorobenzene (3 mL) was added into a 10 mL microwave reaction vessel (CEM Corporation), followed by addition of catalyst CpCo(CO)₂ (2 μL, 0.012 mmol, 0.2 equiv). The reaction vessel was sealed and purged with nitrogen, then the resulting solution was subjected to microwave irradiation at 300 W, 180° C., for 15 min. The volatile components were removed in vacuo and the residue was purified by flash chromatography (hexanes:EtOAc, 1:1, R_{f} 0.25) on silica gel to yield cyclization product 6a (21.0 mg, 81% yield) as a brown solid: mp 110° C.; [α]²⁵_{D} +100.8 (c 1.40, CHCl₃); ¹H NMR (400 MHz, CDCl₃) δ 7.44-7.31 (overlap, 5H), 7.27 (d, J=8.6 Hz, 2H), 6.86 (d, J=8.6 Hz, 2H), 4.71 (d, J_{AB}=16.4 Hz, 1H), 4.57 (d, J_{AB}=16.4 Hz, 1H), 3.79 (s, 3H), 3.67 (s, 2H), 3.56 (ddq, J=10.4, 2.0, 6.0 Hz, 1H), 3.44 (br s, 2H), 3.02 (dd, J=6.0, 5.6 Hz, 2H), 2.82 (ddd, J=11.6, 5.6, 5.6 Hz, 1H), 2.76 (ddd, J=11.6, 6.0, 6.0 Hz, 1H), 2.63 (dd, J_{AB}=16.4, J_{AX}=10.4 Hz, 1H), 2.49 (dd, J_{AB}=16.4, J_{BX}=2.0 Hz, 1H), 1.25 (d, J=6.0 Hz, 3H); ¹³C NMR (75 MHz, CDCl₃) δ 159.0, 156.3, 151.6, 140.6, 140.1, 130.3 (2C), 130.1, 129.0 (2C), 128.4 (2C), 128.0, 124.3, 124.2, 113.9 (2C), 70.6, 65.4, 62.1, 55.4, 51.5, 50.0, 34.4, 32.7, 21.5; HRLCMS (ESI) m/z 401.2216 ([M+1]⁺, 48%) calcd for C₂₆H₂₉N₂O₂ 401.2229.

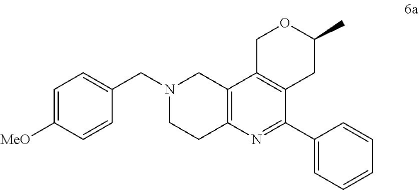

(S)-2-(4-Methoxybenzyl)-8-methyl-6-(phenoxymethyl)-2,3,4,7,8,10-hexahydro-1H-pyrano[4,3-c][1,6]naphthyridine (6b): Compound 6b was prepared according to Procedure D, as described above, beginning with dialkynylnitrile 5b (30.0 mg, 0.070 mmol) and CpCo(CO)₂ (2 μL, 0.014 mmol, 0.2 eq). Purification by flash chromatography (hexanes:EtOAc, 1:1, R_{f} 0.27) gave 6b (26.7 mg, 0,062 mmol, 87% yield) as a sticky yellow oil: [α]²⁵_{D} +51.6 (c 5.66, CHCl₃); ¹H NMR (400 MHz, CDCl₃) δ 7.27-7.22 (overlap, 4H), 6.97 (dd, J=8.8, 1.2 Hz, 2H), 6.92 (tt, J=7.4, 1.2 Hz, 1H), 6.85 (d, J=8.4 Hz, 2H), 5.09 (s, 2H), 4.64 (d, J_{AB}=16.3 Hz, 1H), 4.52 (d, J_{AB}=16.3 Hz, 1H), 3.79 (s, 3H), 3.70 (overlap, 1H), 3.65 (br s, 2H), 3.40 (br s, 2H), 3.00 (dd, J=5.9, 5.9 Hz, 2H), 2.87-2.78 (overlap, 2H), 2.76 (ddd, J_{AB}=11.8, J=5.9, 5.9 Hz, 1H), 2.64 (dd, J_{AB}=16.8, J_{AX}=10.6 Hz, 1H), 1.31 (d, J=6.4 Hz, 3H); ¹³C NMR (75 MHz, CDCl₃) δ 158.9, 158.7, 151.2, 151.1, 140.7, 130.2 (2C), 130.0, 129.4 (2C), 126.5, 125.5, 121.0, 114.8 (2C), 113.8 (2C), 70.3, 70.1, 65.0, 62.0, 55.2, 51.4, 49.7, 32.4, 31.8, 21.5; HRLCMS (ESI) m/z 431.2307 ([M+1]⁺, 33%) calcd for C₂₇H₃₁N₂O₃ 431.2335.

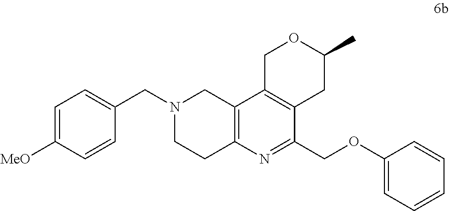

(S)-6-Butyl-2-(4-methoxybenzyl)-8-methyl-2,3,4,7,8,10-hexahydro-1H-pyrano[4,3-c][1,6]naphthyridine (6c): Compound 6c was prepared according to Procedure D, as described above, beginning with dialkynylnitrile 5c (27.0 mg, 0.070 mmol) and CpCo(CO)₂ (2 μL, 0.014 mmol, 0.2 eq). Purification by flash chromatography (hexanes:EtOAc, 1:1, R_{f} 0.17) gave 6c (23.4 mg, 87% yield) as a white solid: mp 69° C.; [α]²⁵_{D} +60.5 (c 9.76, CHCl₃); ¹H NMR (400 MHz, CDCl₃) δ 7.24 (d, J=8.4 Hz, 2H), 6.84 (d, J=8.4 Hz, 2H), 4.61 (d, J_{AB}=16.5 Hz, 1H), 4.49 (d, J_{AB}=16.5 Hz, 1H), 3.78 (s, 3H), 3.66 (ddq, J=10.6, 2.2, 6.3 Hz, 1H), 3.64 (br s, 2H), 3.36 (br s, 2H), 2.96 (br dd, J=5.9, 5.8 Hz, 2H), 2.82 (ddd, J=11.6, 5.8, 5.8 Hz, 1H), 2.71 (ddd, J=11.6, 5.9, 5.9 Hz, 1H), 2.74-2.61 (overlap, 3H), 2.49 (dd, J_{AB}=16.4, J_{BX}=10.6 Hz, 1H), 1.54 (tq, J=7.3, 7.2 Hz, 2H), 1.38 (tt, J=7.2, 7.2 Hz, 2H), 1.34 (d, J=6.3 Hz, 3H), 0.90 (t, J=7.3 Hz, 3H); ¹³C NMR (75 MHz, CDCl₃) δ 159.0, 158.0, 150.8, 139.9, 130.3 (2C), 130.2, 123.8, 122.7, 113.9 (2C), 70.5, 65.3, 62.2, 55.4, 51.5, 50.0, 34.6, 32.6, 32.5, 31.8, 23.1, 21.8, 14.2; LCMS (ESI), m/z (%) 381 ([M+1]⁺, 61); HRLCMS (ESI) m/z 381.2507 ([M+1]⁺, 60%) calcd for C₂₄H₃₃N₂O₂ 381.2542.

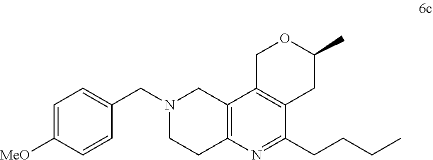

(S)-2-(4-Methoxybenzyl)-8-methyl-6-(phenoxymethyl)-2,3,4,7,8,10-hexahydro-1H-pyrano[4,3-c][1,6]naphthyridine (6d): Compound 6d was prepared according to Procedure D, as described above, beginning with dialkynylnitrile 5d (30.4 mg, 0.060 mmol) and CpCo(CO)₂ (2 μL, 0.012 mmol, 0.2 eq). Purification by flash chromatography (hexanes:EtOAc, 1:1, R_{f} 0.22) gave 6d (27.6 mg, 0.055 mmol, 91% yield) as a sticky brown oil: [α]²⁵_{D} +55.0 (c 0.95, CHCl₃); ¹H NMR (400 MHz, CDCl₃) δ 7.32-7.23 (overlap, 7H), 7.01 (br d, J=8.4 Hz, 1H), 6.87 (d, J=8.2 Hz, 2H), 6.74-6.68 (overlap, 2H), 4.87 (d, J_{AB}=16.3 Hz, 1H), 4.73 (d, J_{AB}=16.3 Hz, 1H), 4.52 (m, 1H), 3.79 (s, 3H), 3.75 (s, 3H), 3.68 (br s, 2H)^{a}, 3.47 (br s 2H)^{a}, 3.02 (br s 2H)^{a}, 2.84 (br m, 1H)^{a}, 2.76 (br m, 1H)^{a}, 2.70-2.40 (br m, 2H)^{a}, 2.04 (br s, 3H)^{a}; ¹³C NMR (75 MHz, CDCl₃) δ 159.4, 159.1, 157.0, 151.4, 141.6, 140.2, 137.3, 132.0, 130.4 (2C), 130.0, 129.8, 128.6 (2C), 127.9, 126.0 (2C), 125.1, 123.9, 115.9, 113.9 (2C), 111.3, 76.4, 65.7, 62.2, 55.4, 55.3, 51.6, 49.9, 33.6 (br)^{a}, 32.7, 20.0; HRLCMS (ESI) m/z 507.2617 ([M+1]⁺, 100%) calcd for C₃₃H₃₅N₂O₃ 507.2648 (^{a}Peaks were very broad presumably due to slow rotation about C-6 aryl C—C bond).

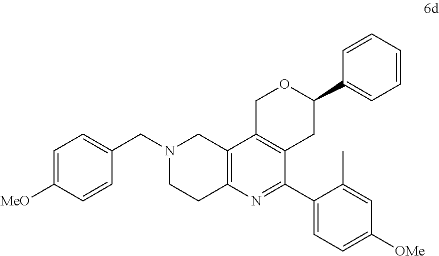

### Example 6

### Procedure E

### Deprotection of PMB Group (6→1, 1a-1d) (Trost, et al., 1996)⁵

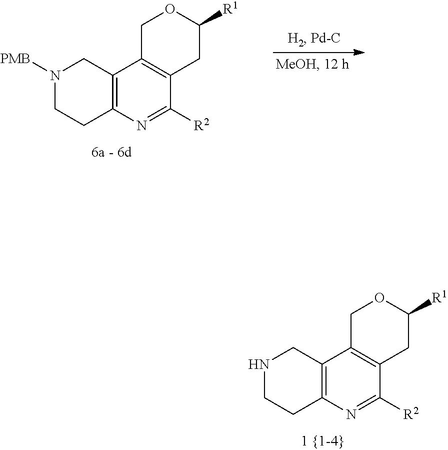

(S)-8-Methyl-6-phenyl-2,3,4,7,8,10-hexahydro-1H-pyrano[4,3-c][1,6]naphthyridine (1{1}): To a solution of the p-methoxybenzyl protected 5,6,7,8-tetrahydro-1,6-naphthyridine 6a (800 mg, 2.0 mmol) in MeOH (10 mL), an equal weight of 20% Pd—C and HOAc (6 μL, 5 mol %) were added. The solution was stirred under a hydrogen atmosphere at room temperature, monitored by TLC. After completion of the reaction (12 h), the catalyst was removed by filtration through a short silica pad, eluting with MeOH (50 mL). The solvent was removed in vacuo and the residue was purified by flash chromatography (CH₂Cl₂: MeOH, 5:1, R_{f} 0.35) on silica gel to afford 1{1} as a sticky brown oil (398.6 mg, 71% yield): [α]²⁵_{D} +102.0 (c 0.60, CHCl₃); IR (NaCl) 2931, 1569, 1426, 1127, 752 cm⁻¹; ¹H NMR (400 MHz, CDCl₃) δ 7.45-7.32 (overlap, 5H), 4.76 (d, J_{AB}=16.4 Hz, 1H), 4.62 (d, J_{AB}=16.4 Hz, 1H), 4.58 (br, NH), 3.97 (d, J_{AB}=16.4 Hz, 1H), 3.89 (d, J_{AB}=16.4 Hz, 1H), 3.59 (br dq, J=10.6, 6.0 Hz, 1H), 3.30 (m, 2H), 3.08 (dd, J=5.8, 5.6 Hz, 2H), 2.65 (dd, J_{AB}=16.2, J_{AX}=10.2 Hz, 1H), 2.51 (br d, J_{AB}=16.2 Hz, 1H), 1.27 (d, J=6.0 Hz, 3H); ¹³C NMR (75 MHz, CD₃OD, ¹³C NMR spectrum in CDCl₃ suffered from peak overlap; all peaks were resolved in CD₃OD) δ 157.1, 148.6, 142.5, 138.9, 128.6 (2C), 128.2, 128.0 (2C), 125.8, 121.0, 70.3, 64.6, 41.3, 40.9, 33.8, 28.7, 20.2; HRLCMS (ESI) m/z 281.1640 ([M+1]⁺, 100%) calcd for C₁₈H₂₁N₂O 281.1654.

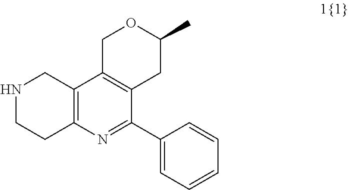

(S)-8-Methyl-6-(phenoxymethyl)-2,3,4,7,8,10-hexahydro-1H-pyrano[4,3-c][1,6]naphthyridine (1{2}): Compound 1{2} was prepared according to Procedure E, as described above, beginning with tetrahydronaphthyridine 6b (867.5 mg, 2.0 mmol). Purification by flash chromatography (CH₂Cl₂:MeOH, 5:1, R_{f} 0.71) gave 1{2} as a white solid (412.7 mg, 1.33 mmol, 68% yield): mp 71° C.; [α]²⁵_{D} +71.4 (c 3.88, CHCl₃); IR (NaCl) 3284, 2932, 1559, 1585, 1496, 1238, 1128, 754 cm⁻; ¹H NMR (400 MHz, CD₃CN, ¹H NMR spectrum in CDCl₃ showed severe broadening) δ 7.28 (dd, J=8.8, 7.3 Hz, 2H), 7.01 (dd, J=8.8, 1.1 Hz, 2H), 6.94 (tt, J=7.3, 1.1 Hz, 1H), 5.09 (d, J_{AB}=11.0 Hz, 1H), 5.05 (d, J_{AB}=11.0 Hz, 1H), 4.68 (d, J_{AB}=16.4 Hz, 1H), 4.54 (d, J_{AB}=16.4 Hz, 1H), 3.74 (br d, J_{AB}=16.6 Hz, 1H), 3.68 (ddq, J=10.8, 2.8, 5.9 Hz, 1H), 3.63 (br d, J_{AB}=16.6 Hz, 1H), 3.04 (m, 2H), 2.82 (dd, J_{AB}=16.6, J_{AX}=2.8 Hz, 1H), 2.78 (dd, J=5.9, 5.7 Hz, 2H), 2.57 (dd, J_{AB}=16.6, J_{BX}=10.8 Hz, 1H), 2.28 (br s, NH), 1.27 (d, J=5.9 Hz, 3H); ¹³C NMR (75 MHz, CDCl₃) δ 158.8, 151.3, 151.2, 140.7, 129.5 (2C), 126.7, 126.5, 121.0, 114.8 (2C), 70.3, 70.2, 65.0, 43.9, 43.6, 32.6, 31.8, 21.6; HRLCMS (ESI) m/z 311.1767 ([M+1]⁺, 12%) calcd for C₁₉H₂₃N₂O₂ 311.1760.

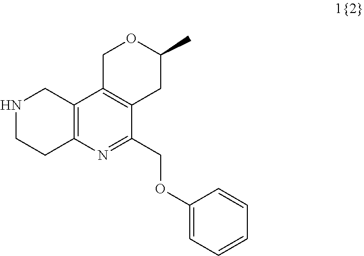

(S)-6-Butyl-8-methyl-2,3,4,7,8,10-hexahydro-1H-pyrano[4,3-c][1,6]naphthyridine (1{3}): Compound 1{3} was prepared according to Procedure E, as described above, beginning with tetrahydronaphthyridine 6c (651.7 mg, 2.1 mmol). Purification by flash chromatography (CH₂Cl₂:MeOH, 5:1, R_{f} 0.39) gave 1(3) as a sticky light yellow oil (334.5 mg, 1.29 mmol 63% yield): [α]²⁵_{D} +103.7 (c 1.93, CHCl₃); IR (NaCl) 2956, 1577, 1429, 1130, 827, 753 cm⁻¹; ¹H NMR (400 MHz, CD₃OD, ¹H NMR spectrum in CDCl₃ showed severe broadening) δ 4.72 (d, J_{AB}=16.5 Hz, 1H), 4.60 (d, J_{AB}=16.5 Hz, 1H), 3.87 (br d, J_{AB}=16.0 Hz, 1H), 3.77 (d, J_{AB}=16.0 Hz, 1H), 3.75 (ddq, J=10.8, 2.8, 5.8 Hz, 1H), 3.20 (br m, 2H), 2.92 (dd, J=6.0, 5.6 Hz, 2H), 2.75 (dd, J_{AB}=16.6, J_{AX}=2.8 Hz, 1H), 2.70 (m, 2H), 2.50 (dd, J_{AB}=16.6, J_{BX}=10.8 Hz, 1H), 1.57 (m, 2H), 1.42 (tq, J=7.3, 7.3 Hz, 2H), 1.34 (d, J=5.8 Hz, 3H), 0.94 (t, J=7.3 Hz, 3H); ¹³C NMR (75 MHz, CDCl₃, inverse gated NMR) δ 158.2, 150.4, 140.0, 124.3, 122.8, 70.5, 65.2, 43.2 (2C), 34.5, 32.4, 32.2, 31.6, 23.1, 21.7, 14.1; HRLCMS (ESI) m/z 261.1946 ([M+1]⁺, 28%) calcd for C₁₆H₂₅N₂O 261.1967.

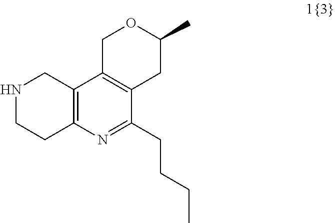

(R)-6-(4-Methoxy-2-methylphenyl)-8-phenyl-2,3,4,7,8,10-hexahydro-1H-pyrano[4,3-c][1,6]naphthyridine (1{4}): Compound 1{4} was prepared according to Procedure E, as described above, beginning with tetrahydronaphthyridine 6{4} (912.2 mg, 1.8 mmol). Purification by flash chromatography (CH₂Cl₂:MeOH, 5:1, R_{f} 0.75) gave 1(4) as a light yellow solid (368.8 mg, 0.97 mmol, 53% yield): mp 151° C.; [α]²⁵_{D} +53.6 (c 0.97, CHCl₃); IR (NaCl) 2927, 2840, 1576, 1426, 1242, 1111, 753 cm⁻¹; ¹H NMR (400 MHz, CD₃CN, ¹H NMR spectrum in CDCl₃ and CD₃OD showed severe broadening) δ 7.34-7.24 (overlap, 5H), 7.04 (d, J=8.4 Hz, 1H), 6.83 (d, J=2.4 Hz, 1H), 6.75 (dd, J=8.4, 2.4 Hz, 1H), 4.92 (d, J_{AB}=16.1 Hz, 1H), 4.79 (d, J_{AB}=16.1 Hz, 1H), 4.61 (m, 1H), 3.89 (d, J_{AB}=16.4 Hz, 1H), 3.79 (d, J_{AB}=16.4 Hz, 1H), 3.77 (s, 3H), 3.14 (m, 2H), 2.86 (dd, J=5.9, 5.6 Hz, 2H), 2.59 (br m, 1H), 2.48 (br m, 1H), 2.30 (br s, NH), 2.05 (s, 3H); ¹³C NMR (75 MHz, CD₃OD, ¹³C NMR spectrum in CDCl₃ showed severe broadening) δ 161.4, 158.6, 151.3, 143.7, 143.0, 138.7, 132.3, 131.0, 129.6 (2C), 128.9, 127.9, 127.0 (2C), 124.8, 116.9, 112.5, 77.3, 66.5, 55.8, 43.6, 35.0, 31.4, 20.0 (one sp³ carbon missing in ¹³C NMR spectrum in CD₃OD due to overlap with solvent appears at δ 43.8 in CDCl₃); HRLCMS (ESI) m/z 387.2101 ([M+1]⁺, 100%) calcd for C₂₅H₂₇N₂O₂ 387.2073.

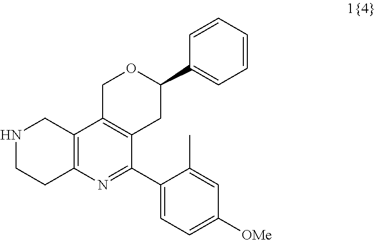

### Example 7

### Procedure F

### Preparation of Urea Sublibrary 7 (1→7)

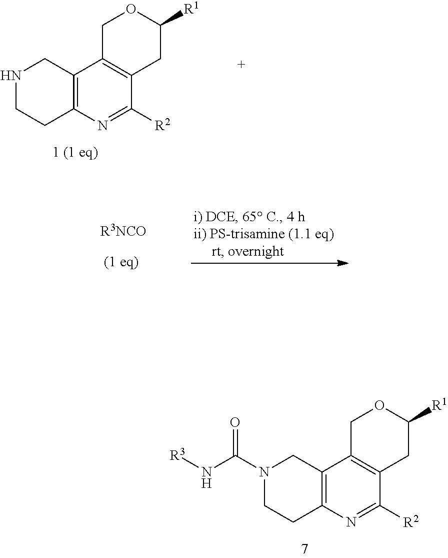

A MiniBlock XT reaction block hosting 48 reactor-tubes was used for library preparation. Stock solutions of scaffolds 1{1-4} in anhydrous dichloromethane were prepared (5 mg/mL). Each scaffold was treated with eight isocyanates (Table 11) as follows: 3.0 mL of the scaffold stock solution (15.0 mg, 1.0 equiv) and isocyanate (1.1 equiv) were placed into the reactor-tube. The 4×8 reaction vessels in the Miniblock synthesizer were then heated to 65° C. for 4 hours with stirring. PS-Trisamine (1.0 equiv, 0.13 g, Argonaut Technologies Inc., P/N 800229; Lot No. 03307; 0.446 mmol/g) was then added into each reaction tube, and the reaction mixture was stirred at 50° C. overnight (12 h). The PS-trisamine resin was removed by filtration, and the filtrate was collected and transferred into a high throughput centrifugal evaporator (Genevac) to evaporate to dryness. The crude residue of each reaction was purified by mass-directed LCMS to provide the library members 7{1-4, 1-8}. Representative examples are given as below.

(S)-Methyl 3-methyl-2-((S)-8-methyl-6-phenyl-2,3,4,7,8,10-hexahydro-1H-pyrano[4,3-c][1,6]naphthyridine-2-carboxamido)butanoate (7{1,3}): Compound 7(1,3) was prepared according to Procedure F; as described above (18.3 mg, 0.042 mmol, 78% yield). [α]²⁵_{D} +66.6 (c 0.90, CHCl₃); ¹H NMR (400 MHz, CDCl₃) δ 7.36-7.44 (overlap, 5H), 5.08 (d, J=8.4 Hz, NH), 4.84 (d, J_{AB}=16.4 Hz, 1H), 4.69 (d, J_{AB}=16.4 Hz, 1H), 4.44 (overlap, 3H), 3.72 (s, 3H), 3.70-3.64 (overlap m, 2H), 3.60 (m, 1H), 3.07 (m, 2H), 2.66 (dd, J_{AB}=16.6, J=10.4 Hz, 1H), 2.66 (dd, J_{AB}=16.6, J=2.0 Hz, 1H), 2.14 (m, 1H), 1.27 (d, J=6.0 Hz, 3H), 0.95 (d, J=6.8 Hz, 3H), 0.91 (d, J=6.8 Hz, 3H); ¹³C NMR (100 MHz, CDCl₃) δ 174.0, 157.4, 156.8, 151.0, 141.0, 139.9, 129.1 (2C), 128.6 (2C), 128.3, 125.3, 123.0, 70.7, 65.4, 58.8, 52.4, 42.1, 41.8, 34.5, 32.3, 31.6, 21.6, 19.3, 18.3; HRLCMS (ESI) m/z 438.2398 ([M+1]⁺, 100%) calcd for C₂₅H₃₂N₃O₄ 438.2393.

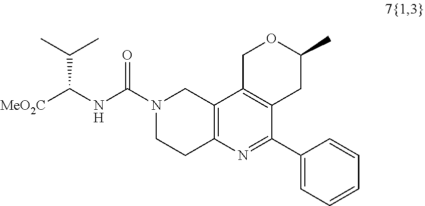

(S)-8-Methyl-6-(phenoxymethyl)-N—((S)-1-phenylethyl)-3,4,7,8-tetrahydro-1H-pyrano[4,3-c][1,6]naphthyridine-2(10H)-carboxamide (7{2,1}): Compound 7{2,1} was prepared according to Procedure F, as described above (20.1 mg, 0.044 mmol, 91% yield). [α]²⁵_{D} +76.7 (c 0.75, CHCl₃); ¹H NMR (400 MHz, CDCl₃) δ 7.24-7.10 (overlap, 7H), 6.88 (d, J=8.4 Hz, 2H), 6.85 (t, J=7.2 Hz, 1H), 4.99 (s, 2H), 4.93 (qd, J=6.4, 6.8 Hz, 1H), 4.72 (d, J=6.8 Hz, NH), 4.66 (d, J_{AB}=16.6 Hz, 1H), 4.53 (d, J_{AB}=16.6 Hz, 1H), 4.30 (d, J_{AB}=16.6 Hz, 1H), 4.23 (d, J_{AB}=16.6 Hz, 1H), 3.61 (br m, 1H), 3.55 (ddd, J_{AB}=13.2, J=6.6, 6.6 Hz, 1H), 3.48 (ddd, J_{AB}=13.2, J=6.6, 6.6 Hz, 1H), 2.92 (br s, 2H), 2.74 (br d, J_{AB}=16.0 Hz, 1H), 2.57 (dd, J_{AB}⁼16.0, J=10.8 Hz, 1H), 1.40 (d, J=6.8 Hz, 3H), 1.23 (d, J=6.4 Hz, 3H); ¹³C NMR (75 MHz, CDCl₃) δ 158.8, 156.8, 151.7, 150.6, 144.3, 141.4, 129.7 (2C), 128.9 (2C), 127.6, 127.5, 126.3 (2C), 124.5, 121.3, 114.9 (2C), 70.3, 70.1, 65.2, 50.5, 41.9, 41.7, 32.0, 31.9, 22.7, 21.7; HRLCMS (ESI) m/z 458.2463 ([M+1]⁺, 100%) calcd for C₂₈H₃₂N₃O₃ 458.2444.

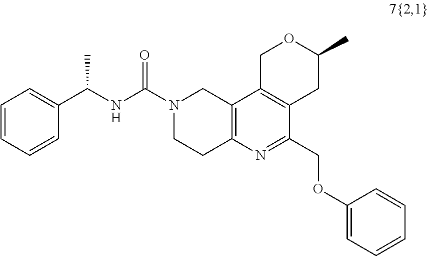

(S)-Ethyl 4-(6-Butyl-8-methyl-2,3,4,7,8,10-hexahydro-1H-pyrano[4,3-c][1,6]naphthyridine-2-carboxamido)butanoate (7{3,4}): Compound 7{3,4} was prepared according to Procedure F, as described above (17.5 mg, 0.042 mmol, 73% yield). [α]²⁵_{D} +42.5 (c 0.87, CHCl₃); IR (NaCl) 3346, 2930, 1731, 1628, 1536, 1261, 1176 cm⁻¹; ¹H NMR (400 MHz, CDCl₃) δ 5.13 (t, J=5.0 Hz, NH), 4.75 (d, J_{AB}=16.2 Hz, 1H), 4.60 (d, J_{AB}=16.2 Hz, 1H), 4.29 (s, 2H), 4.08 (q, J=7.2 Hz, 2H), 3.70 (m, 1H), 3.62 (ddd, J_{AB}=13.2, J=5.6, 5.6 Hz, 1H), 3.57 (ddd, J_{AB}=13.2, J=5.6, 5.6 Hz, 1H), 3.28 (dt, J=5.0, 6.4 Hz, 2H), 2.93 (dd, J=5.6, 5.6 Hz, 2H), 2.67-2.63 (overlap, 3H), 2.51 (dd, J_{AB}=16.2, J=10.6 Hz, 1H), 2.37 (t, J=6.6 Hz, 2H), 1.84 (tt, J=6.6, 6.4 Hz, 2H), 1.55 (tq, J=7.2, 7.2 Hz, 2H), 1.38 (overlap, 2H), 1.35 (d, J=6.0 Hz, 3H), 1.21 (t, J=7.2 Hz, 3H), 0.90 (t, J=7.2 Hz, 3H); ¹³C NMR (100 MHz, CDCl₃) δ 174.4, 158.4, 157.7, 150.4, 140.3, 124.7, 121.6, 70.6, 65.3, 60.8, 41.7, 41.6, 41.1, 34.6, 32.6, 32.4, 32.2, 31.6, 24.9, 23.2, 21.8, 14.4, 14.2; HRLCMS (ESI) m/z 440.2540 ([M+Na]⁺, 81%) calcd for C₂₃H₃₅N₃O₄Na 440.2525.

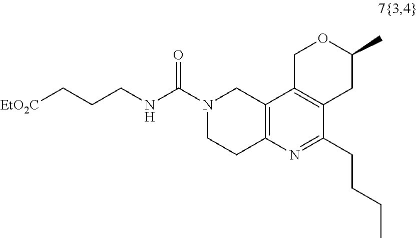

(S)-6-Butyl-N-(furan-2-ylmethyl)-8-methyl-3,4,7,8-tetrahydro-1H-pyrano[4,3-c][1,6]naphthyridine-2(10H)-carboxamide (7{3,5}): Compound 7{3,5} was prepared according to Procedure F, as described above (18.1 mg, 0.047 mmol, 82% yield). [α]²⁵_{D} +49.0 (c 0.90, CHCl₃); ¹H NMR (400 MHz, CDCl₃) δ 7.31 (dd, J=2.4, 0.8 Hz, 1H), 6.28 (dd, J=3.2, 2.4 Hz, 1H), 6.20 (br d, J=3.2 Hz, 1H), 4.96 (t, J=5.2 Hz, NH), 4.72 (d, J_{AB}=16.4 Hz, 1H), 4.59 (d, J_{AB}=16.4 Hz, 1H), 4.41 (d, J=5.2 Hz, 2H), 4.32 (s, 2H), 3.69 (m, 1H), 3.61 (ABXX′, J_{AB}=13.2, J_{Ax}=J_{AX′}=5.6 Hz, 1H), 3.59 (ABXX′, J_{AB}=13.2, J_{BX}=J_{BX′}=6.0 Hz, 1H), 2.94 (dd, J=6.0, 5.6 Hz, 2H), 2.69-2.61 (overlap, 3H), 2.51 (dd, J_{AB}=16.4, J=10.4 Hz, 1H), 1.55 (qt, J=7.2, 7.2 Hz, 2H), 1.42-1.32 (overlap, 2H), 1.35 (d, J=6.0 Hz, 3H), 0.90 (t, J=7.2 Hz, 3H); ¹³C NMR (100 MHz, CDCl₃) δ 158.4, 157.3, 152.5, 150.2, 142.3, 140.6, 125.0, 121.6, 110.6, 107.5, 70.6, 65.3, 41.8, 41.7, 38.2, 34.4, 32.5, 32.0, 31.6, 23.1, 21.8, 14.2; HRLCMS (ESI) m/z 384.2279 ([M+1]⁺, 33%) calcd for C₂₂H₃₀N₃O₃ 384.2287.

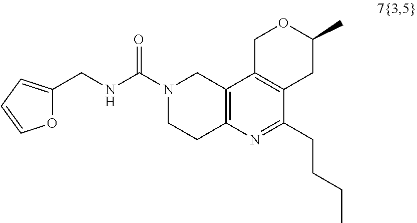

(S)-6-Butyl-N-(3-cyanophenyl)-8-methyl-3,4,7,8-tetrahydro-1H-pyrano[4,3-c][1,6]naphthyridine-2(10H)-carboxamide (7{3,6}): Compound 7{3,6} was prepared according to Procedure F, as described above (21.0 mg, 0.052 mmol, 90% yield). [α]²⁵_{D} +34.1 (c 0.59, CHCl₃); ¹H NMR (400 MHz, CDCl₃) δ 7.75 (br s, 1H), 7.64 (ddd, J=8.2, 1.2, 0.8 Hz, 1H), 7.36 (dd, J=8.2, 8.2 Hz, 1H), 7.31 (dd, J=8.2, 0.8 Hz, 1H), 6.76 (br s, NH), 4.79 (d, J_{AB}=16.4 Hz, 1H), 4.65 (d, J_{AB}=16.4 Hz, 1H), 4.45 (s, 2H), 3.82-3.72 (overlap, 3H), 3.07 (dd, J=6.0, 5.2 Hz, 2H), 2.73-2.67 (overlap, 3H), 2.56 (dd, J_{AB}=16.0, J=10.4 Hz, 1H), 1.55 (tq, J=8.0, 7.2 Hz, 2H), 1.48-1.38 (overlap, 2H), 1.40 (d, J=6.4 Hz, 3H), 0.95 (t, J=7.2 Hz, 3H); ¹³C NMR (75 MHz, CDCl₃) δ 158.9, 154.5, 149.9, 140.4, 140.1, 130.0, 126.8, 125.1, 124.5, 123.2, 121.0, 119.0, 112.9, 70.6, 65.3, 42.4, 41.9, 34.6, 32.5, 32.2, 31.6, 23.2, 21.8, 14.2; HRLCMS (ESI) m/z 405.2320 ([M+1]⁺, 100%) calcd for C₂₄H₂₉N₄O₂ 405.2291.

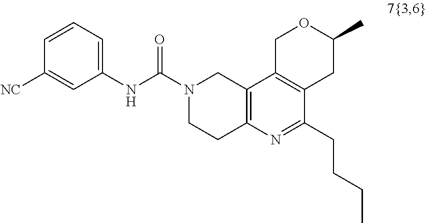

(R)-6-(4-Methoxy-2-methylphenyl)-N-(4-methoxyphenyl)-8-phenyl-3,4,7,8-tetrahydro-1H-pyrano[4,3-c][1,6]naphthyridine-2(10H)-carboxamide (7{4,2}): Compound 7{4,2} was prepared according to Procedure F, as described above (14.0 mg, 0.026 mmol, 67% yield). [α]²⁵_{D} +29.0 (c 0.60, CHCl₃); ¹H NMR (400 MHz, CDCl₃) δ 7.32-7.20 (overlap, 7H), 7.03 (d, J=8.0 Hz, 1H), 6.83 (d, J=7.2 Hz, 2H), 6.76-6.70 (overlap, 2H), 6.45 (s, NH), 5.01 (d, J_{AB}=16.4 Hz, 1H), 4.85 (d, J_{AB}=16.4 Hz, 1H), 4.59-4.50 (overlap, 3H), 3.84-3.75 (overlap, 2H), 3.76 (s, 6H), 3.11 (dd, J=6.0, 5.6 Hz, 2H), 2.66 (br m, 1H), 2.54 (br m, 1H), 2.06 (s, 3H); ¹³C NMR (75 MHz, CDCl₃) δ 159.6, 157.6, 156.4, 155.7, 150.8, 141.4, 140.7, 137.4, 131.8, 131.6, 129.8, 128.7 (2C), 128.2, 126.3, 126.1 (2C), 122.9 (2C), 122.8, 116.1, 114.4 (2C), 111.5, 76.6, 65.9, 55.7, 55.4, 42.5, 42.0, 33.8, 32.3, 20.1; HRLCMS (ESI) m/z 536.2571 ([M+1]⁺, 100%) calcd for C₃₃H₃₄N₃O₄ 536.2549.

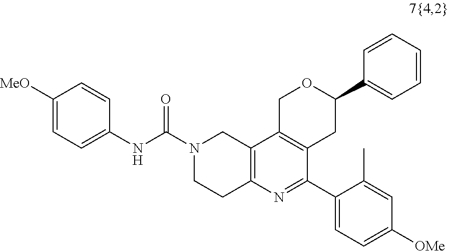

### Example 8

### Procedure G

### Preparation of Amide Sub-library 8 (1→8)

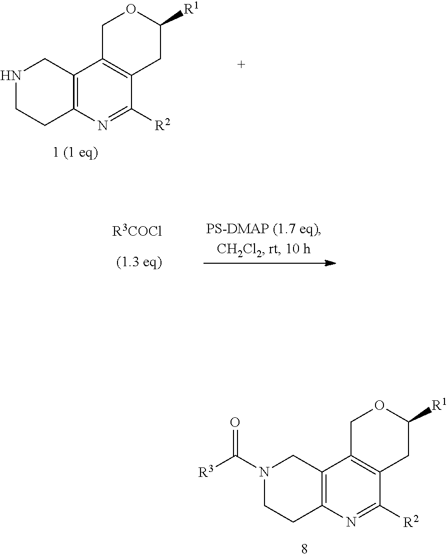

A MiniBlock synthesizer hosting 48 reactor-tubes was used for library preparation. Stock solutions of scaffolds 1{1-4} in anhydrous dichloromethane were prepared (5 mg/mL). Each scaffold was treated with eight acid chlorides (Table 2) as follows: 3.0 mL of the scaffold stock solution (15.0 mg, 1.0 equiv), acid chloride (1.3 equiv) and PS-DMAP resin (1.7 equiv, 0.23 g, Argonaut Technologies Inc., P/N 800290; Lot No. 02899; 0.35 mmol/g). The 4×8 reaction vessels in the Miniblock synthesizer were placed in a mechanical shaker for 10 hours at room temperature. The PS-DMAP resin was removed by filtration, and the filtrate was collected and transferred into a high throughput centrifugal evaporator (Genevac) to evaporate to dryness. The crude residue of each reaction was then purified by LCMS to provide the library members 8{1-4, 1-8}. Representative examples are given as below.

(S)-2-Methoxy-1-(8-methyl-6-phenyl-3,4,7,8-tetrahydro-1H-pyrano[4,3-c][1,6]naphthyridin-2(10H)-yl)ethanone (8{1,1}): Compound 8{1,1} was prepared according to Procedure G, as described above (15.8 mg, 0.045 mmol, 85% yield). [α]²⁵_{D} +52.8 (c 0.8, CHCl₃); ¹H NMR (400 MHz, CDCl₃, rotamer ratio=4:1 determined by ¹H NMR spectrum integration) δ 7.42-7.30 (overlap, 5H, major and minor rotamers), 4.82 (d, J_{AB}=16.4 Hz, 0.8H, major rotamer), 4.78 (overlap, 0.2H, minor rotamer), 4.66 (d, J_{AB}=16.4 Hz, 0.8H, major rotamer), 4.64 (d, J_{AB}=17.2 Hz, 0.2H, minor rotamer), 4.53 (d, J_{AB}=18.0 Hz, 0.8H, major rotamer), 4.50 (d, J_{AB}=18.0 Hz, 0.8H, major rotamer), 4.49 (d, J_{AB}=17.2 Hz, 0.2H, minor rotamer), 4.40 (d, J_{AB}=17.2 Hz, 0.2H, minor rotamer), 4.17 (s, 1.6H, major rotamer), 4.15 (s, 0.4H, minor rotamer), 3.89 (m, 0.4H, minor rotamer), 3.77 (ddd, J_{AB}=13.1, J=6.4, 6.0 Hz, 0.8H, major rotamer), 3.73 (ddd, J_{AB}=13.1, J=6.4, 6.0 Hz, 0.8H, major rotamer), 3.56 (m, 1H, major and minor rotamers), 3.40 (s, 2.4H, major rotamer), 3.37 (s, 0.6H, minor rotamer), 3.04 (dd, J=6.4, 6.0 Hz, 1.6H, major rotamer), 3.00 (overlap, 0.4H, minor rotamer), 2.64 (overlap, 0.2H, minor rotamer), 2.63 (dd, J_{AB}=16.2, J=10.6 Hz, 0.8H, major rotamer), 2.50 (overlap, 0.2H, minor rotamer), 2.48 (br d, J_{AB}=16.2 Hz, 0.8H, major rotamer), 1.24 (d, J=6.4, 3H, major and minor rotamers); ¹³C NMR (100 MHz, CDCl₃) δ 168.5, 156.9, 150.4, 141.5, 139.8, 129.0 (2C), 128.6 (2C), 128.4, 125.5, 122.4, 72.3, 70:7, 65.5, 59.4, 42.8, 40.6, 34.5, 33.0, 21.6; HRLCMS (ESI) m/z 353.1894 ([M+1]⁺, 40%) calcd for C₂₁H₂₅N₂O₃ 353.1865.

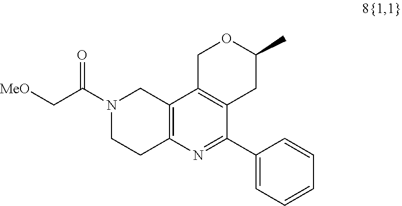

(S)-Cyclohexyl(8-methyl-6-(phenoxymethyl)-3,4,7,8-tetrahydro-1H-pyrano[4,3-c][1,6]naphthyridin-2(10H)-yl)methanone (8{2,6}): Compound 8{2,6} was prepared according to Procedure G, as described above (18.0 mg, 0.043 mmol, 89% yield). [0]²⁵_{D} +45.2 (c 0.90, CHCl₃); ¹H NMR (400 MHz, CDCl₃, rotamer ratio=4:1 determined by ¹H NMR spectrum integration) δ 7.26 (dd, J=8.4, 7.2 Hz, 2H, major and minor rotamers), 6.97 (br d, J=8.4 Hz, 2H, major and minor rotamers), 6.94 (br t, J=7.2 Hz, 1H, major and minor rotamers), 5.08 (s, 2H, major and minor rotamers), 4.78 (d, J_{AB}=16.4 Hz, 1H, major and minor rotamers), 4.64 (d, J_{AB}=16.4 Hz, 1H, major and minor rotamers), 4.55 (d, J_{AB}=17.6 Hz, 0.8H, major rotamer), 4.44 (d, J_{AB}=17.6 Hz, 1H, major and minor rotamers), 4.35 (d, J_{AB}=17.6 Hz, 0.2H, minor rotamer), 3.90 (m, 0.2H, minor rotamer), 3.82 (ddd, J=13.2, 6.0, 6.0 Hz, 0.8H, major rotamer), 3.78-3.66 (overlap, 2H, major and minor rotamers), 3.03 (br m, 1.6H, major rotamer), 2.96 (br m, 0.4H, minor rotamer), 2.83 (br d, J_{AB}=16.4 Hz, 1H, major and minor rotamers), 2.66 (d, J_{AB}=16.4 Hz, J=10.8 Hz, 1H, major and minor rotamers), 2.56 (tt, J=11.5, 2.8 Hz, 0.8H, major rotamer), 2.49 (br m, 0.2H, minor rotamer), 1.84-1.61 (overlap, 6H, major and minor rotamers), 1.60-1.46 (overlap, 2H, major and minor rotamers), 1.33 (d, J=6.0, 3H, major and minor rotamers), 1.28-1.17 (overlap, 2H, major and minor rotamers); ¹³C NMR (100 MHz, CDCl₃) δ 175.5, 158.8, 151.8, 150.0, 141.7, 129.7 (2C), 127.7, 124.5, 121.3, 115.0 (2C), 70.4, 70.2, 65.3, 42.9, 40.9, 40.6, 33.1, 32.0, 29.6, 26.05 (2C), 26.02 (2C), 21.7; HRLCMS (ESI) m/z 421.2471 ([M+1]⁺, 100%) calcd for C₂₆H₃₃N₂O₃ 421.2491.

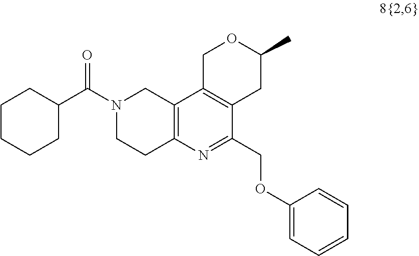

(S)-Cyclopropyl(8-methyl-6-(phenoxymethyl)-3,4,7,8-tetrahydro-1H-pyrano[4,3-c][1,6]naphthyridin-2(10H)-yl)methanone (8{2,8}): Compound 8{2,8} was prepared according to Procedure G, as described above (13.1 mg, 0.035 mmol, 72% yield). [α]²⁵_{D} +33.0 (c 0.50, CHCl₃); ¹H NMR (400 MHz, CDCl₃, rotamer ratio=3:1 determined by ¹H NMR spectrum integration) δ 7.27 (dd, J=8.8, 7.2 Hz, 2H, major and minor rotamers), 6.98 (br d, J=8.8 Hz, 2H, major and minor rotamers), 6.94 (br t, J=7.2 Hz, 1H, major and minor rotamers), 5.10 (s, 2H, major and minor rotamers), 4.78 (d, J_{AB}=16.4 Hz, 1H, major and minor rotamers), 4.64 (br d, J_{AB}=16.4 Hz, 1H, major and minor rotamers), 4.57 (d, J_{AB}=17.6 Hz, 1H, major and minor rotamers), 4.47 (d, J_{AB}=17.6 Hz, 1H, major and minor rotamers), 4.02 (ddd, J_{AB}=14.2, J=8.2, 5.2 Hz, 0.75H, major rotamer), 3.91 (ddd, J_{AB}=14.2, J=6.4, 5.2 Hz, 0.75H, major rotamer), 3.72 (m, 0.75H, major rotamer), 3.68 (overlap, 0.5H, minor rotamer), 3.49 (br m, 0.25H, minor rotamer), 3.08 (br m, 1.5H, major rotamer), 3.04 (br m, 0.5H, minor rotamer), 2.94 (overlap, 0.25H, minor rotamer), 2.83 (br d, J_{AB}=16.4 Hz, 0.75H, major rotamer), 2.67 (dd, J_{AB}=16.4 Hz, J=10.4 Hz, 0.75H, major rotamer), 2.67 (overlap, 0.25H, minor rotamer), 1.84 (m, 0.75H, major rotamer), 1.80 (m, 0.25H, minor rotamer), 1.33 (d, J=6.0, 3H, major and minor rotamers), 1.00 (dd, J=2.8, 2.8 Hz, 2H, major and minor rotamers), 0.81 (overlap, 2H, major and minor rotamers); ¹³C NMR (75 MHz, CDCl₃) δ 172.8, 158.8, 151.8, 150.3, 141.6, 129.7 (2C), 127.2, 124.5, 121.3, 114.9 (2C), 70.4, 70.2, 65.3, 43.1, 41.0, 32.8, 31.9, 21.7, 11.4, 7.8 (2C); HRLCMS (ESI) m/z 379.2021 ([M+1]⁺, 60%) calcd for C₂₃H₂₇N₂O₃ 379.2022.

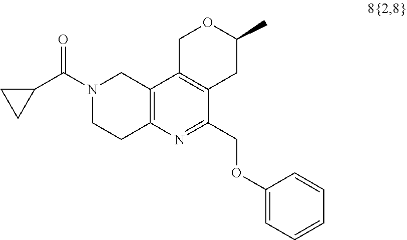

(S)-1-(6-Butyl-8-methyl-3,4,7,8-tetrahydro-1H-pyrano[4,3-c][1,6]naphthyridin-2(10H)-yl)-2-(thiophen-2-yl)ethanone (8{3,2}): Compound 8{3,2} was prepared according to Procedure G, as described above (15.0 mg, 0.039 mmol, 68% yield). [α]²⁵_{D} +53.3 (c 0.75, CHCl₃); ¹H NMR (400 MHz, CDCl₃, rotamer ratio=3:1 determined by ¹H NMR spectrum integration) δ 7.17 (dd, J=5.0, 1.4 Hz, 0.75H, major rotamer), 7.14 (dd, J=4.8, 0.8 Hz, 0.25H, minor rotamer), 6.93-6.88 (overlap, 1.5H, major rotamer), 6.87 (dd, J=4.8, 3.6 Hz, 0.25H, minor rotamer), 6.83 (br d, J=3.6 Hz, 0.25H, minor rotamer), 4.73 (d, J_{AB}=16.4 Hz, 0.75H, major rotamer), 4.59 (d, J_{AB}=16.4 Hz, 1H, major and minor rotamers), 4.50 (overlap, 0.25H, minor rotamer), 4.54 (d, J_{AB}=17.6 Hz, 0.75H, major rotamer), 4.44 (d, J_{AB}=17.6 Hz, 0.75H, major rotamer), 4.37 (d, J_{AB}=16.4 Hz, 0.25H, minor rotamer), 4.29 (d, J_{AB}=16.4 Hz, 0.25H, minor rotamer), 3.99 (s, 1.5H, major rotamer), 3.96 (s, 0.5H, minor rotamer), 3.92 (overlap, 0.25H, minor rotamer), 3.80 (ddd, J_{AB}=13.6, J=6.0, 6.0 Hz, 0.75H, major), 3.75-3.65 (overlap, 2H, major and minor rotamers), 2.93 (br tt, J=6.0 Hz, 0.5H, minor rotamer), 2.88-2.82 (overlap, 1.5H, major rotamer), 2.67-2.63 (overlap, 3H, major and minor rotamers), 2.51 (dd, J_{AB}=16.4, J=10.4 Hz, 1H, major and minor rotamers), 1.55 (overlap, 2H, major and minor rotamers), 1.38 (overlap, 2H, major and minor rotamers), 1.35 (d, J=6.0 Hz, 3H, major and minor rotamers), 0.90 (t, J=7.2 Hz, 3H, major and minor rotamers); ¹³C NMR (100 MHz, CDCl₃) δ 169.2, 158.6, 149.5, 140.7, 136.3, 127.1, 126.4, 125.10, 125.06, 121.1, 70.5, 65.4, 43.9, 40.7, 35.5, 34.6, 32.6, 32.5, 31.5, 23.1, 21.8, 14.2; HRLCMS (ESI) m/z 385.1971 ([M+1]⁺, 100%) calcd for C₂₂H₂₉N₂O₂S 385.1950.

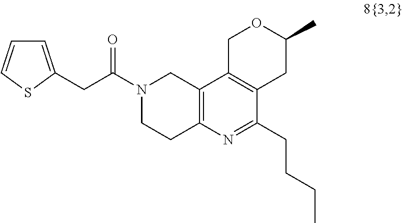

(S)-1-(6-Butyl-8-methyl-3,4,7,8-tetrahydro-1H-pyrano[4,3-c][1,6]naphthyridin-2(10H)-yl)-2-(3-methoxyphenyl)ethanone (8{3,3}): Compound 8{3,3} was prepared according to Procedure G, as described above (18.3 mg, 0.045 mmol, 78% yield). [α]²⁵_{D} +44.9 (c 0.90, CHCl₃); ¹H NMR (400 MHz, CDCl₃, rotamer ratio=4:1 determined by ¹H NMR spectrum integration) δ 7.20 (dd, J=7.8, 7.8 Hz, 0.8H, major rotamer), 7.16 (dd, J=7.6, 7.6 Hz, 0.2H, minor rotamer), 6.85-6.68 (overlap, 3H), 4.74 (d, J_{AB}=16.4 Hz, 0.8H, major rotamer), 4.59 (d, J_{AB}=16.4 Hz, 0.8H, major rotamer), 4.53 (d, J_{AB}=17.4 Hz, 0.8H, major rotamer), 4.46 (overlap, 0.2H, minor rotamer), 4.43 (d, J_{AB}=17.4 Hz, 0.8H, major rotamer), 4.35 (d, J_{AB}=15.6 Hz, 0.2H, minor rotamer), 4.27 (d, J_{AB}=16.6 Hz, 0.2H, minor rotamer), 4.20 (d, J_{AB}=16.6 Hz, 0.2H, minor rotamer), 3.81-3.61 (overlap, 5.6H, major and minor rotamers), 3.75 (s, 2.4H, major rotamer), 2.92 (t, J=6.0 Hz, 0.4H, minor rotamer), 2.80-2.70 (overlap, 1.6H, major and minor rotamers), 2.66-2.58 (overlap 3H, major and minor rotamers), 2.49 (dd, J_{AB}=16.0, J=10.8 Hz, 0.8H, major rotamer), 2.48 (overlap, 0.2H, minor rotamer), 1.54 (overlap, 2H, major and minor rotamers), 1.37 (overlap, 2H, major and minor rotamers), 1.36 (d, J=6.0 Hz, 3H, major and minor rotamers), 0.91 (t, J=7.2 Hz, 0.6H, minor rotamer), 0.90 (t, J=7.2 Hz, 2.4H, major rotamer); ¹³C NMR (100 MHz, CDCl₃) δ 170.2, 160.2, 158.5, 149.6, 140.6, 136.4, 130.0, 125.0, 121.2, 121.1, 114.5, 112.6, 70.5, 65.4, 55.4, 43.8, 41.6, 40.6, 34.6, 32.6, 32.5, 31.5, 23.2, 21.8, 14.2; HRLCMS (ESI) m/z 409.2503 ([M+1]⁺, 75%) calcd for C₂₅H₃₃N₂O₃ 409.2491.

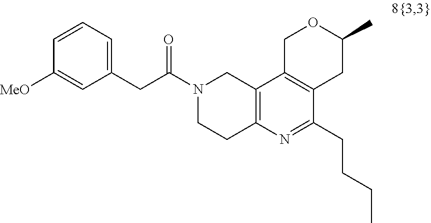

### Example 9

### Procedure H

### Preparation of Sulfonamide Sub-library 9 (1→9)

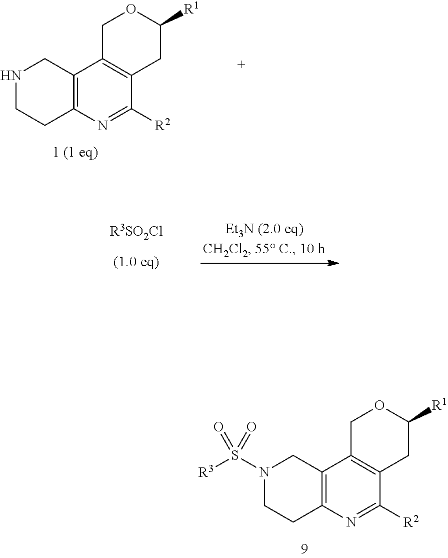

A Miniblock XT miniblock hosting 48 reactor-tubes was used for library preparation. Stock solutions of scaffolds 1{1-4} in anhydrous dichloromethane were prepared (5 mg/mL). Each scaffold was treated with eight sulfonyl chlorides (Table 3) as follows: 3.0 mL of the scaffold stock solution (15.0 mg, 1.0 equiv), sulfonyl chloride (1.0 equiv) and triethylamine (3.0 equiv, 22 μL) were placed into the reactor-tube. The 4×8 reaction vessels in the Miniblock synthesizer were then heated to 55° C. for 10 hours with stirring. The reactor-tubes were then placed into a high throughput centrifugal evaporator (Genevac) to evaporate to dryness. The crude residue of each reaction was then purified by LCMS to provide the library members 9{1-4, 1-8}. Representative examples are given as below.

(S)-2-(3,5-Dimethylisoxazol-4-ylsulfonyl)-8-methyl-6-phenyl-2,3,4,7,8,10-hexahydro-1H-pyrano[4,3-c][1,6]naphthyridine (9{1,5}): Compound 9{1,5} was prepared according to Procedure H, as described above (15.5 mg, 0.035 mmol, 66% yield). [α]²⁵_{D} +33.2 (c 0.75, CHCl₃); ¹H NMR (400 MHz, CDCl₃) 7.46-7.38 (overlap, 5H), 4.76 (d, J_{AB}=16.4 Hz, 1H), 4.66 (d, J_{AB}=16.4 Hz, 1H), 4.19 (AA′, 2H), 3.62 (ddd, J=12.0, 6.0, 6.0 Hz, 1H), 3.65-3.59 (overlap, 1H), 3.54 (ddd, J=12.0, 6.0, 6.0 Hz, 1H), 3.14 (dd, J=6.0, 6.0 Hz, 2H), 2.71 (s, 3H), 2.70 (dd, J_{AB}=16.6, J=10.0 Hz, 1H), 2.56 (dd, J_{AB}=16.6, J=1.8 Hz, 1H), 2.44 (s, 3H), 1.30 (d, J=6.0 Hz, 3H); ¹³C NMR (100 MHz, CDCl₃) δ 174.2, 158.1, 157.5, 149.7, 140.9, 139.5, 129.0 (2C), 128.63 (2C), 128.56, 125.6, 120.8, 114.2, 70.8, 65.1, 43.3, 43.0, 34.4, 32.3, 21.6, 13.2, 11.5; HRLCMS (ESI) m/z 440.1660 ([M+1]⁺, 100%) calcd for C₂₃H₂₆N₃O₄S 440.1664.

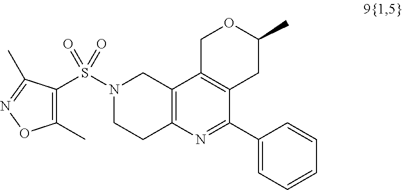

(S)-8-Methyl-2-(4-methyl-3,4-dihydro-2H-benzo[b][1,4]oxazin-7-ylsulfonyl)-6-phenyl-2,3,4,7,8,10-hexahydro-1H-pyrano[4,3-c][1,6]naphthyridine (9{1,7}): Compound 9{1,7} was prepared according to Procedure H, as described above (16.3 mg, 0.033 mmol, 62% yield). [α]²⁵_{D} −124.9 (c 0.7, CHCl₃); ¹H NMR (400 MHz, CDCl₃) 7.42-7.33 (overlap, 5H), 7.10 (d, J=8.4 Hz, 1H), 7.01 (s, 1H), 6.82 (d, J=8.4 Hz, 1H), 4.74 (d, J_{AB}=16.4 Hz, 1H), 4.62 (d, J_{AB}⁼16.4 Hz, 1H), 4.31 (dd, J=4.0, 3.6 Hz, 2H), 4.07 (d, J_{AB}=15.6 Hz, 1H), 4.02 (d, J_{AB}=15.6 Hz, 1H), 3.57 (br m, 1H), 3.50 (ddd, J=12.2, 6.0, 6.0 Hz, 1H), 3.33 (ddd, J=12.2, 6.0, 6.0 Hz, 1H), 3.28 (dd, J=4.0, 3.6 Hz, 2H), 3.09 (br dd, J=6.0, 6.0 Hz, 2H), 2.91 (s, 3H), 2.64 (dd, J_{AB}=16.4, J=10.4 Hz, 1H), 2.50 (br d, J_{AB}=16.4 Hz, 1H), 1.26 (d, J=6.0 Hz, 3H); ¹³C NMR (100 MHz, CDCl₃) δ 157.0, 150.4, 148.2, 140.9, 139.7, 137.0, 129.0 (2C), 128.5 (2C), 128.4, 128.3, 125.2, 121.5, 118.3, 116.2 111.1, 70.7, 65.3, 65.2, 48.5, 43.85, 43.82, 38.9, 34.4, 32.4, 21.6; HRLCMS (ESI) m/z 492.1941 ([M+1]⁺, 100%) calcd for C₂₇H₃₀N₃O₄S 492.1957.

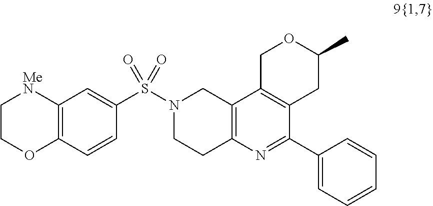

(S)-8-Methyl-2-(4-methyl-3,4-dihydro-2H-benzo[b][1,4]oxazin-7-ylsulfonyl)-6-(phenoxymethyl)-2,3,4,7,8,10-hexahydro-1H-pyrano[4,3-c][1,6]naphthyridine (9{2,7}): Compound 9{2,7} was prepared according to Procedure H, as described above (17.1 mg, 0.033 mmol, 68% yield). [α]²⁵_{D} −106.5 (c 0.85, CHCl₃); ¹H NMR (400 MHz, CDCl₃) 7.27 (t, J=7.6 Hz, 2H), 7.11 (dd, J=8.0, 1.0 Hz, 1H), 7.03 (br s, 1H), 7.00-6.94 (overlap, 3H), 6.84 (dd, J=8.0, 1.0 Hz, 1H), 5.09 (s, 2H), 4.69 (d, J_{AB}=16.4 Hz, 1H), 4.60 (d, J_{AB}=16.4 Hz, 1H), 4.33 (dd, J=4.4, 3.6 Hz, 2H), 4.07 (d, J_{AB}=15.6 Hz, 1H), 3.99 (d, J_{AB}=15.6 Hz, 1H), 3.72 (ddq, J=10.8, 0.8, 6.0 Hz, 1H), 3.54 (ddd, J=12.0, 6.0, 6.0 Hz, 1H), 3.35-3.26 (overlap, 3H), 3.08 (ABX₂, 2H), 2.93 (s, 3H), 2.84 (dd, J_{AB}=16.4, J=0.8 Hz, 1H), 2.66 (dd, J_{AB}=16.4, J=10.8 Hz, 1H), 1.35 (d, J=6.0 Hz, 3H); ¹³C NMR (100 MHz, CDCl₃) δ 158.8, 152.2, 149.8, 148.2, 141.1, 137.0, 129.7 (2C), 128.3, 127.4, 122.9, 121.3, 118.3, 116.2, 114.9 (2C), 111.1, 70.4, 70.3, 65.2, 65.1, 48.5, 43.8, 43.7, 38.9, 32.2, 31.9, 21.7; HRLCMS (ESI) m/z 522.2057 ([M+1]⁺, 100%) calcd for C₂₈H₃₂N₃O₅S 522.2063.

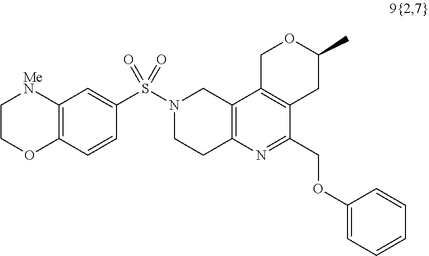

(S)-6-Butyl-8-methyl-2-(4-methylbenzenesulfonyl)-2,3,4,7,8,10-hexahydro-1H-pyrano[4,3-c][1,6]naphthyridine (9{3,1}): Compound 9{3,1} was prepared according to Procedure H, as described above (17.1 mg, 0.041 mmol, 72% yield). [α]²⁵_{D} +30.5 (c 0.85, —CHCl₃); ¹H NMR (400 MHz, CDCl₃) δ 7.69 (d, J=7.8 Hz, 2H), 7.30 (d, J=7.8 Hz, 2H), 4.62 (d, J_{AB}=16.2 Hz, 1H), 4.53 (d, J_{AB}=16.2 Hz, 1H), 3.99 (d, J_{AB}=15.2 Hz, 1H), 3.92 (d, J_{AB}⁼15.2 Hz, 1H), 3.66 (m, 1H), 3.48 (ddd, J=11.2, 5.6, 5.6 Hz, 1H), 3.27 (dddd, J=11.2, 5.6, 5.6 Hz, 1H), 2.99 (m, 2H), 2.68-2.60 (overlap, 3H), 2.48 (dd, J_{AB}=16.4, J=10.4 Hz, 1H), 2.40 (s, 3H), 1.53 (tt, J=7.8, 7.2, 2H), 1.36 (qt, J=7.2, 7.2 Hz, 2H), 1.35 (d, J=6.0 Hz, 3H), 0.89 (t, J=7.4 Hz, 3H); ¹³C NMR (100 MHz, CDCl₃) δ 158.9, 149.3, 144.1, 140.2, 133.4, 130.0 (2C), 127.9 (2C), 124.8, 119.9, 70.6, 65.1, 43.8, 43.7, 34.6, 32.5, 32.2, 31.5, 23.1, 21.77, 21.75, 14.2; HRLCMS (ESI) m/z 415.2046 ([M+1]⁺, 100%) calcd for C₂₃H₃₁N₂O₃S 415.2055.

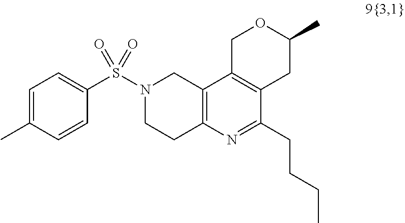

(S)—N-(4-(6-Butyl-8-methyl-3,4,7,8-tetrahydro-1H-pyrano[4,3-c][1,6]-naphthyridin-2(10H)-ylsulfonyl)phenyl)acetamide (9{3,2}): Compound 9{3,2} was prepared according to Procedure H, as described above (26.0 mg, 0.057 mmol, 99+% yield). [α]²⁵_{D} +20.8 (c 0.6, CHCl₃); ¹H NMR (400 MHz, CDCl₃) δ 7.75 (d, J_{AB}=8.8 Hz, 2H), 7.66 (d, J_{AB}=8.8 Hz, 2H), 7.54 (br s, NH), 4.62 (d, J_{AB}=16.0 Hz, 1H), 4.52 (d, J_{AB}=16.0 Hz, 1H), 3.98 (d, J_{AB}=15.2 Hz, 1H), 3.93 (d, J_{AB}=15.2 Hz, 1H), 3.67 (m, 1H), 3.46 (ddd, J=12.0, 5.9, 5.7 Hz, 1H), 3.28 (ddd, J=12.0, 5.9, 5.7 Hz, 1H), 2.99 (dd, J=5.9, 5.7 Hz, 2H), 2.63 (t, J=7.8 Hz, 2H), 2.65-2.61 (overlap, 1H), 2.49 (dd, J_{AB}=16.4, J=10.4 Hz, 1H), 2.18 (s, 3H), 1.54 (tt, J=7.8, 7.2 Hz, 2H), 1.40-1.32 (qt, J=6.0, 7.2 Hz, 2H), 1.35 (d, J=6.0 Hz, 3H), 0.89 (t, J=7.2 Hz, 3H); ¹³C NMR (100 MHz, CDCl₃) δ 168.8, 159.0, 149.2, 142.6, 140.2, 131.1, 129.1 (2C), 124.9, 119.8, 119.6 (2C), 70.6, 65.1, 43.8, 43.6, 34.6, 32.4, 32.1, 31.5, 24.9, 23.1, 21.8, 14.2; HRLCMS (ESI) m/z 458.2123 ([M+1]⁺, 100%) calcd for C₂₄H₃₂N₃O₄S 458.2114.

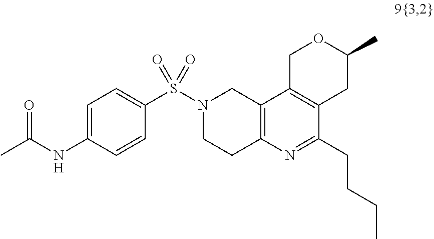

(R)-6-(4-Methoxy-2-methylphenyl)-8-phenyl-2-(4-methylbenzenesulfonyl)-2,3,4,7,8,10-hexahydro-1H-pyrano[4,3-c][1,6]naphthyridine (9{4,1}): Compound 9{4,1} was prepared according to Procedure H, as described above (13.2 mg, 0.024 mmol, 63% yield). [α]²⁵_{D} +26.2 (c 0.65, CHCl₃); ¹H NMR (400 MHz, CDCl₃) δ 7.75 (br m, 2H), 7.34-722 (overlap, 7H), 6.98 (br m, 1H), 6.75-6.60 (overlap, 2H), 4.90 (br d, J_{AB}=16.4 Hz, 1H), 4.78 (d, J_{AB}=16.4 Hz, 1H), 4.53 (br m, 1H)^{a}, 4.13 (br d, J_{AB}=15.6 Hz, 1H), 4.06 (d, J_{AB}=15.6 Hz, 1H), 3.75 (s, 3H), 3.55 (br m, 1H), 3.35 (br m, 1H), 3.08 (br m, 2H)^{a}, 2.62 (br m, 1H)^{a}, 2.52 (br m, 1H)^{a}, 2.41 (s, 3H), 2.00 (br s, 3H); ¹³C NMR (100 MHz, CDCl₃) δ 159.6, 157.9, 150.0, 144.2, 141.4, 140.6, 137.3, 133.4, 131.6, 130.1 (2C), 129.7, 128.7 (2C), 128.2, 128.0 (2C), 127.3, 126.1 (2C), 121.1, 116.1, 111.6, 76.5, 65.6, 55.4, 43.8, 42.2, 33.7, 32.3, 21.8, 20.0; HRLCMS (ESI) m/z 541.2190 ([M+1]⁺, 100%) calcd for C₃₂H₃₃N₂O₄S 541.2161. (Note: ^{a}peaks are very broad presumably due to slow rotation about C-6 aryl C—C bond).

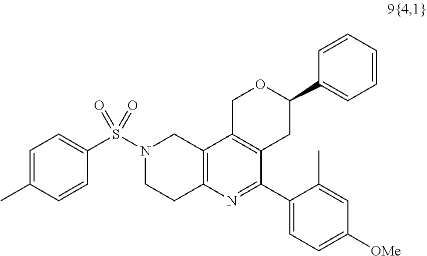

(R)—N-(4-(6-(4-Methoxy-2-methylphenyl)-8-phenyl-3,4,7,8-tetrahydro-1H-pyrano[4,3-c][1,6]naphthyridin-2(10H)-ylsulfonyl)phenyl)acetamide (9{4,2}): Compound 9{4,2} was prepared according to Procedure H, as described above (22.6 mg, 0.039 mmol, 99+% yield). [α]²⁵_{D} −75.3 (c 1.20, CHCl₃); ¹H NMR (400 MHz, CDCl₃) 7.78 (d, J_{AB}=8.4 Hz, 2H), 7.68 (d, J_{AB}=8.6 Hz, 2H), δ 7.63 (br s, NH), 7.33-7.25 (overlap, 5H), 6.99 (d, J=8.6 Hz, 1H), 6.72-6.69 (overlap, 2H), 4.89 (d, J_{AB}=16.4 Hz, 1H), 4.78 (d, J_{AB}=16.4 Hz, 1H), 4.54 (m, 1H)^{a}, 4.11 (d, J_{AB}=15.2 Hz, 1H), 4.09 (d, J_{AB}=15.2 Hz, 1H), 3.74 (s, 3H), 3.54 (ddd, J=12.0, 5.6, 5.6 Hz, 1H), 3.37 (ddd, J=12.0, 5.6, 5.6 Hz, 1H), 3.08 (br t, 2H)^{a}, 2.63 (br m, 1H)^{a}, 2.52 (br m, 1H)^{a}, 2.18 (s, 3H), 2.00 (s, 3H); ¹³C NMR (100 MHz, CDCl₃) δ 168.8, 159.6, 158.0, 149.9, 142.6, 141.3, 140.7, 137.3, 131.5, 131.2, 129.7, 129.2 (2C), 128.7 (2C), 128.2, 126.3, 126.1 (2C), 121.1, 119.6 (2C), 116.1, 111.6, 76.5, 65.6, 55.4, 43.8, 33.7, 32.2, 29.9, 24.9, 20.0; HRLCMS (ESI) m/z 584.2274 ([M+1]⁺, 100%) calcd for C₃₃H₃₄N₃O₅S 584.2219. (Note: ^{a}peaks are very broad presumably due to slow rotation about C-6 aryl C—C bond),

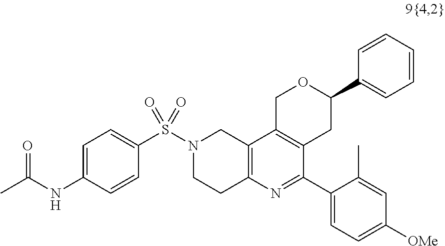

### Example 10

### Procedure I

### Preparation of Tertiary Amines

### Reductive Amination (1→10)

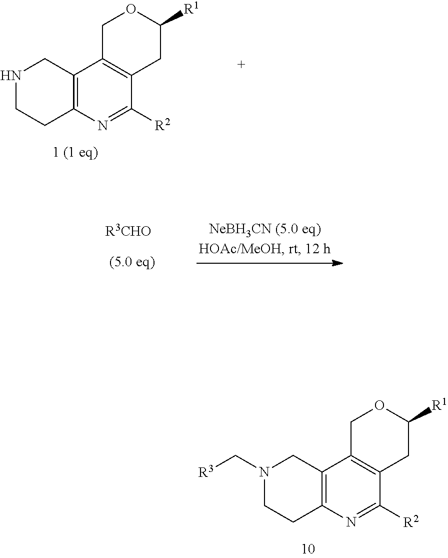

A solution of scaffold 1{1-4} (13.0-14.0 mg, 1.0 equiv) in MeOH (1.5 mL) was placed in a reaction vial. To this solution was added aldehyde (5.0 equiv) in MeOH (0.5 mL), followed by the addition of NaBH₃CN (5.0 equiv). The pH of the reaction was adjusted to 6 by adding HOAc. The reaction mixture was stirred at room temperature overnight (12 h), then quenched with saturated NaHCO₃ solution (5.0 mL), and extracted with EtOAc (3×10 mL). The organic layers were combined and washed with brine, then dried over Na₂SO₄. The solvent was removed in vacuo and the crude residue was purified by flash chromatography on silica gel to give the desired product 10a-10e (Table 4). Representative examples are given as below.

(8S)-2-((6,6-Dimethylbicyclo[3.1.1]hept-2-en-3-yl)methyl)-8-methyl-6-phenyl-2,3,4,7,8,10-hexahydro-1H-pyrano[4,3-c][1,6]naphthyridine (10b): Compound 10b was prepared according to Procedure I, as described above, beginning with 1{1} (14.0 mg, 0.050 mmol), (1R)-(−)-myrtenal (38 μl, 0.250 mmol, 5.0 equiv) and NaBH₃CN (16.0 mg, 0.250 mmol, 5.0 equiv). Purification by flash chromatography on silica gel gave 10b as a sticky yellow oil (hexanes:EtOAc, 1:1, R_{f} 0.67; 16.1 mg, 78% yield): [α]²⁵_{D} +68.0 (c 0.44, CHCl₃); ¹H NMR (400 MHz, CDCl₃) δ 7.45-7.32 (overlap, 5H), 5.45 (br s, 1H), 4.75 (d, J_{AB}=16.5 Hz, 1H), 4.61 (d, J_{AB}=16.5 Hz, 1H), 3.57 (br m, 1H), 3.39 (d, J_{AB}=15.8 Hz, 1H), 3.33 (d, J_{AB}=15.8 Hz, 1H), 3.12-2.97 (overlap, 4H), 2.83 (ddd, J=11.3, 5.6, 5.6 Hz, 1H), 2.71 (ddd, J=11.3, 5.8, 5.8 Hz, 1H), 2.63 (dd, J_{AB}=15.8, J_{AX}=10.6 Hz, 1H), 2.49 (br d, J_{AB}=15.8 Hz, 1H), 2.38 (ddd, J=8.4, 5.5, 5.5 Hz, 1H), 2.34-2.20 (overlap, 3H), 2.09 (br s, 1H), 1.26 (d, J=6.0 Hz, 3H), 1.25 (s, 3H), 1.12 (d, J=8.4 Hz, 1H), 0.84 (s, 3H); ¹³C NMR (75 MHz, CDCl₃) δ 156.3, 151.8, 145.6, 140.6, 140.2, 129.1 (2C), 128.5 (2C), 128.1, 124.6, 124.4, 120.7, 70.7, 65.5, 64.2, 51.8, 50.5, 44.5, 41.2, 38.2, 34.5, 32.6, 32.2, 31.6, 26.4, 21.6, 21.3; HRLCMS (ESI) m/z 415.2780 ([M+1]⁺, 100%) calcd for C₂₈H₃₅N₂O 415.2749.

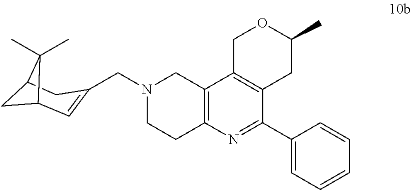

(S)-6-Butyl-2-(3,4-dimethoxybenzyl)-8-methyl-2,3,4,7,8,10-hexahydro-1H-pyrano[4,3-c][1,6]naphthyridine (10d): Compound 10d was prepared according to Procedure I, as described above, beginning with 6c (13.0 mg, 0.050 mmol), 3,4-dimethoxybenzaldehyde (42.0 mg, 0.250 mmol, 5.0 eq) and NaBH₃CN (16.0 mg, 0.250 mmol, 5.0 eq). Purification by flash chromatography (hexanes:EtOAc, 1:1, R_{f} 0.08) gave 10d as a sticky light yellow oil (18.0 mg, 88% yield): [α]²⁵_{D} +70.4 (c 0.23, CHCl₃); ¹H NMR (400 MHz, CDCl₃) δ 6.91 (d, J=1.6 Hz, 1H), 6.85 (dd, J=8.1, 1.6 Hz, 1H), 6.79 (d, J=8.1 Hz, 1H), 4.63 (d, J_{AB}=16.1 Hz, 1H), 4.51 (d, J_{AB}=16.1 Hz, 1H), 3.85 (s, 3H), 3.84 (s, 3H), 3.67 (ddq, J=10.8, 2.2, 5.9 Hz, 1H), 3.62 (s, 2H), 3.36 (br s, 2H), 2.95 (dd, J=5.9, 5.5 Hz, 2H), 2.80 (ddd, J=11.2, 5.5, 5.5 Hz, 1H), 2.72-2.64 (overlap, 3H), 2.64 (overlap dd, J_{AB}=16.0, J_{AX}=2.2 Hz, 1H), 2.50 (dd, J_{AB}=16.0, J_{BX}=10.8 Hz, 1H), 1.57 (tq, J=7.0, 7.3 Hz, 2H), 1.39 (tt, J=7.3, 7.3 Hz, 2H), 1.35 (d, J=5.9 Hz, 3H), 0.91 (d, J=7.3 Hz, 3H); ¹³C NMR (75 MHz, CDCl₃) δ 157.8, 150.3, 149.3, 148.6, 141.3, 130.4, 124.6, 123.2, 121.4, 112.2, 111.1, 70.5, 65.3, 62.4, 56.1 (2C), 51.4, 49.7, 34.2, 32.4, 31.9, 31.7, 23.1, 21.8, 14.2; HRLCMS (ESI) m/z 411.2677 ([M+1]⁺, 100%) calcd for C₂₅H₃₅N₂O₃ 411.2648.

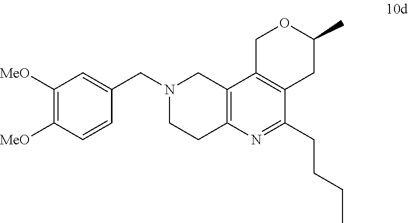

### Example 11

### Biological Activity of Library Compounds Against Mycobacterium tuberculosis

The library compounds of ureas 7, amides 8, and sulfonamides 9 were screened for activity against Mycobacterium tuberculosis using the microplate Alamar Blue assay (MABA), described in Falzari, et al., Antimicrob. Agents Chemother. 2005, 49, 1447-1454 (2005), content of which is herein incorporated by reference. The MIC values were established after initial positive hits were identified (>80% inhibition at 10 μg/mL). Cytotoxicity assays were performed on Vero cells with IC₅₀ values greater than 50 μg/mL under the assay conditions. Activities were subsequently validated in duplicate assays with resynthesized compound.

Library compounds 7{4,1} 7{4,2} and 9{4,4}, all with the 6-(4-methoxy-4-methyl)phenyl and 8-phenyl substituents of scaffold 1{4} and N2 derivatized as an aryl-bearing urea or sulfonamide, showed significant activities. The MIC and IC₅₀ values of these compounds are shown in Scheme 5.

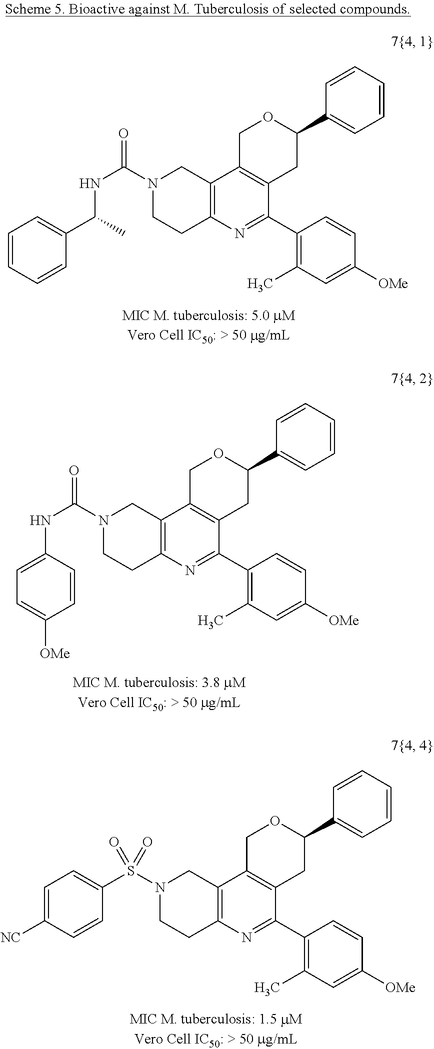

As shown in this example, the compounds of the invention can serve as new drugs candidates for a new treatment of tuberculosis. These compounds of the invention are structurally unique to anything in use today, and as such, should not have drug resistance issues with the current M. tuberculosis strains. Furthermore, cytotoxicty studies with Vero cells have shown these compounds to be completely non-cytotoxic at 50 μg/mL, with >80% inhibition of M. tuberculosis growth at only 10 μg/mL. Thus, a good therapeutic window should exist for the compounds to be used as anti-tuberculosis drug candidates. In addition, given their unique structures, the compounds of the invention could well have other significant biological activities against other diseases and disorders of medical value.

The present invention can be defined by any of the following numbered paragraphs:
1. A compound of formula III or IV:

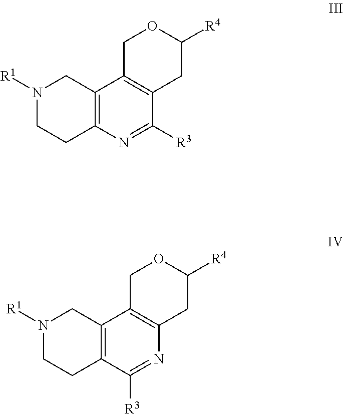

wherein:
R¹ is —(CH₂)ₘR⁶, —C(O)N(H)R⁶, —C(O)R⁶, or—S(O)₂R⁶;
m is 0-6;
R³ and R⁴ are each independently a substituted or unsubstituted alkyl, substituted or unsubstituted aryl, or substituted or unsubstituted heterocylic ring containing one or more heteroatoms; and
R⁶ represents independently for each occurrence a substituted or unsubstituted alkyl, substituted or unsubstituted cycloalkyl, substituted or unsubstituted alkenyl, substituted or unsubstituted cycloalkenyl, substituted or unsubstituted aryl, substituted or unsubstituted alkoxy, substituted, or unsubstituted aryloxy, substituted or unsubstituted heterocylic ring containing one or more heteroatoms, or substituted or unsubstituted fused ring formed between two or more cyclic rings or heteroatom-containing cyclic rings.
2. The compound of paragraph 1, wherein the compound is represented by formula III.
3. The compound of paragraph 1, wherein R¹ is —(CH₂)ₘR⁶, m is 0-6, and R⁶ represents independently for each occurrence a substituted C₂-C₁₂ alkenyl, substituted C₅-C₁₀ cycloalkenyl with an alkylene bridge connecting two carbons on the ring, substituted aryl, or unsubstituted 5-member heterocylic ring containing one or more heteroatoms, wherein the heteroatoms are O, N or S.
4. The compound of paragraph 3, wherein the substituted alkenyl or cycloalkenyl is an alkenyl or cycloalkenyl group substituted with one or more alkyl groups; and the substituted aryl is a phenyl group substituted with one or more alkoxy or aryloxy groups.
5. The compound of paragraph 3, wherein m is 1; and R⁶ is in each occurrence independently selected from the group consisting of

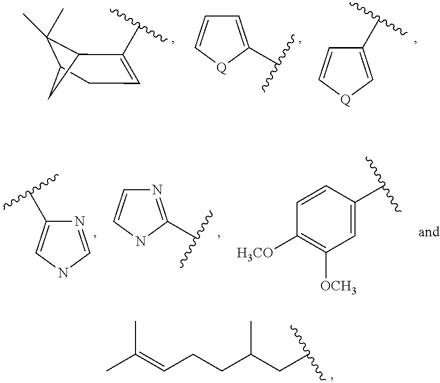

wherein Q is O, S, or N.
6. The compound of paragraph 1, wherein R¹ is —C(O)N(H)R⁶, and R⁶ represents independently for each occurrence a substituted C₁-C₆ alkyl, unsubstituted C₂-C₆ alkenyl, substituted aryl, or unsubstituted 5-member heterocylic ring containing one or more heteroatoms, wherein the heteroatoms are O, N or S.
7. The compound of paragraph 6, wherein the substituted alkyl is an alkyl group substituted with one or more moieties selected from the group consisting of alkyl, phenyl, indolyl, furanyl, thiofuranyl, pyrrolyl, imidazolyl, and —C(O)OR⁸, wherein R⁸ is hydrogen, or substituted or unsubstituted C₁-C₆ alkyl; and
   the substituted aryl is a phenyl group substituted with one or more moieties selected from the group consisting of alkoxy, aryloxy and cyano.
8. The compound of paragraph 6, wherein R⁶ is in each occurrence independently selected from the group consisting of

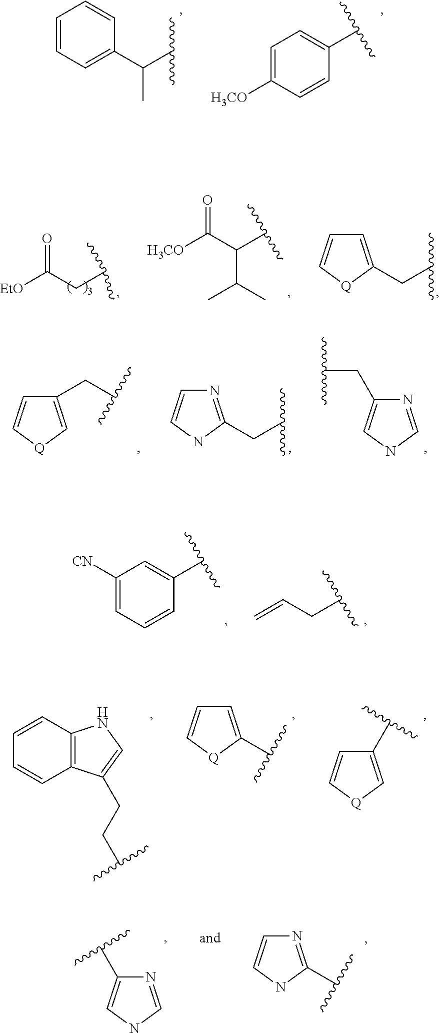

wherein Q is O, S, or N.
9. The compound of paragraph 1, wherein R¹ is —C(O)R⁶, and R⁶ represents independently for each occurrence a substituted C₁-C₆ alkyl, unsubstituted C₃-C₈ cycloalkyl, substituted aryl, or unsubstituted 5- or 6-member heterocylic ring containing one or more heteroatoms, wherein the heteroatoms are O, N or S.
10. The compound of paragraph 9, wherein the substituted alkyl is an alkyl group substituted with one or more moieties selected from the group consisting of alkoxy, aryloxy, phenyl, furanyl, thiofuranyl, pyrrolyl, and imidazolyl, which is optionally substituted with one or more alkoxy or aryloxy groups; and the substituted aryl is a phenyl group substituted with one or more alkyl or halo groups.
11. The compound of paragraph 9, wherein R⁶ is in each occurrence independently selected from the group consisting of methoxyethyl,

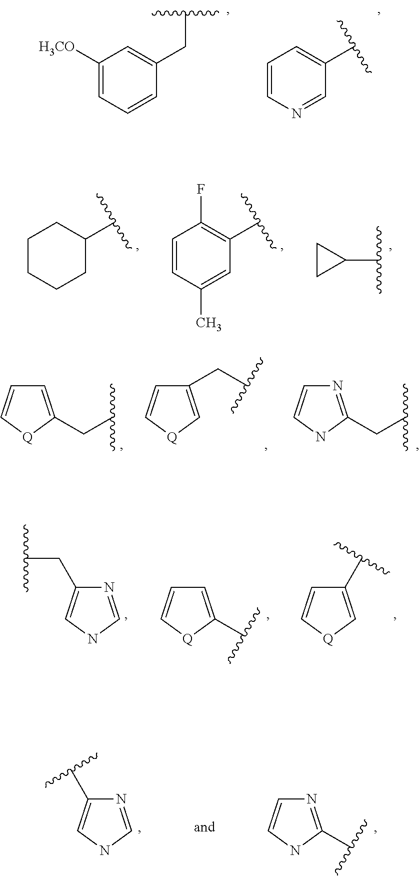

wherein Q is O, S, or N.
12. The compound of paragraph 1, wherein R¹ is —S(O)₂R⁶, and R⁶ represents independently for each occurrence an unsubstituted C₁-C₆ alkyl, substituted aryl, substituted or unsubstituted 5-member heterocylic ring containing one or more heteroatoms, or substituted fused ring formed between a cyclic ring and a heterocyclic ring containing one or more heteroatoms, wherein the heteroatoms are O, N or S.
13. The compound of paragraph 12, wherein the substituted aryl is a phenyl group substituted with one or more moieties selected from the group consisting of alkyl, cyano and

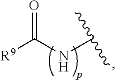

wherein R⁹ is alkyl or alkoxy, and p is 0 or 1; the substituted 5-member heterocylic ring is

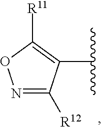

wherein R¹¹ and R¹² are each independently alkyl or alkoxy; and the substituted fused ring is

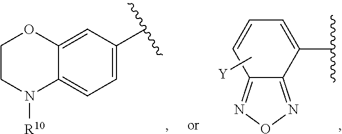

wherein R¹¹ is alkyl or alkoxy, and Y is a halide.
14. The compound of paragraph 12, wherein R⁶ is in each occurrence independently selected from the group of n-butyl,

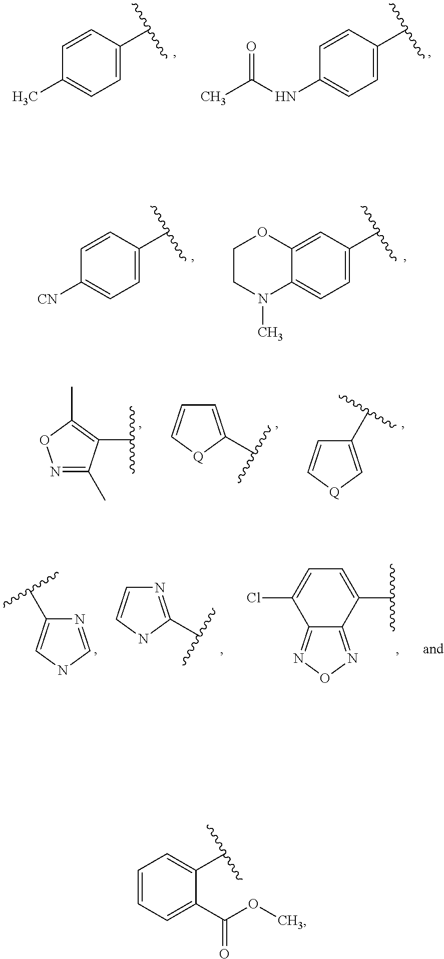

wherein Q is O, S, or N.
15. The compound of paragraph 1, wherein R³ and R⁴ are each independently a substituted or unsubstituted C₁-C₆ alkyl, or substituted or unsubstituted aryl.
16. The compound of paragraph 15, wherein R³ is an unsubstituted C₁-C₆ alkyl, unsubstituted phenyl, substituted C₁-C₆ alkyl, wherein the substituted alkyl is an alkyl group substituted with one or more moieties selected from the group consisting of alkyl, alkoxy and aryloxy, or substituted phenyl, wherein the substituted phenyl is a phenyl group substituted with one or more moieties selected from the group consisting of alkyl, alkoxy and aryloxy.
17. The compound of paragraph 16, wherein R³ is selected from the group consisting of n-butyl, phenyl,

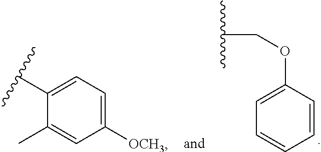

18. The compound of paragraph 15, wherein R⁴ is an unsubstituted C₁-C₆ alkyl, unsubstituted phenyl, or substituted C₁-C₆ alkyl, wherein the substituted alkyl is an alkyl group substituted with one or more moieties selected from the group consisting of alkyl, alkoxy and aryloxy, which is further optionally substituted with alkoxy or aryloxy.
19. The compound of paragraph 18, wherein R⁴ is selected from the group consisting of methyl, phenyl or

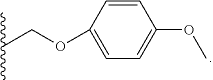

20. The compound of paragraph 1, represented by formula (IIIa):

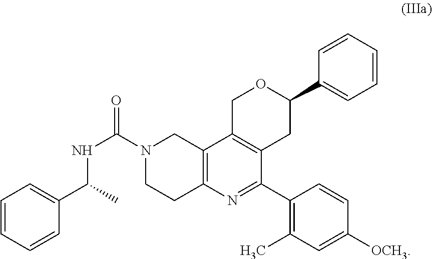

21. The compound of paragraph 1, represented by formula (IIIb):

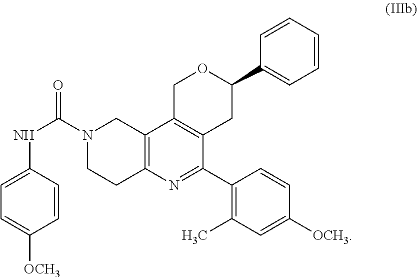

22. The compound of paragraph 1, represented by formula (IIIc):

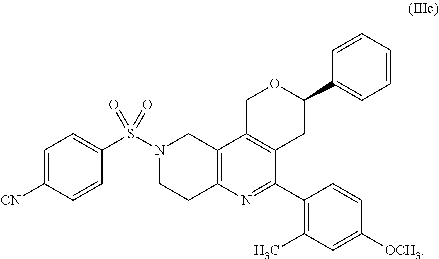

23. A pharmaceutical composition, comprising a therapeutically effective amount of a compound of any of paragraphs 1-23 and a pharmaceutically acceptable carrier or excipient.
24. A method for treating tuberculosis, bacteria infection caused by tuberculosis, or related diseases, which comprises administering a therapeutically effective amount of a compound of formula I or II, or a salt thereof to a subject in need of such treatment:

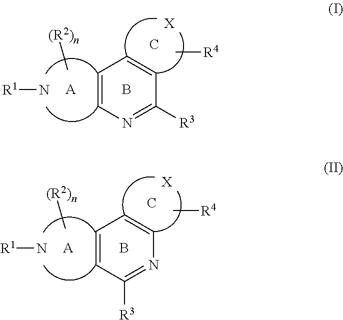

wherein
A and C are each independently a 5-7 member heterocyclic ring, wherein the N and X variables are present at any position in the ring;
R¹ is H, substituted or unsubstituted alkyl, substituted or unsubstituted aryl, —(CH₂)ₘR⁶, —C(O)N(H)R⁶, —C(O)R⁶, or —S(O)₂R⁶;
m is 0-6;
each R² is independently H, alkyl, or aryl;
n is 0-4;
R³ is hydrogen, substituted or unsubstituted alkyl, substituted or unsubstituted aryl, or substituted or unsubstituted heterocylic ring containing one or more heteroatoms;
R⁴ is hydrogen, substituted or unsubstituted alkyl, substituted or unsubstituted aryl, or substituted or unsubstituted heterocylic ring containing one or more heteroatoms, wherein R⁴ is in an ortho-position to X on the heterocyclic ring and with the proviso that R⁴ is H when C is a 5-member ring;
X is O, S, S(O), S(O)₂, or NR⁵, wherein R⁵ is substituted or unsubstituted alkyl or aryl; and
R⁶ represents independently for each occurrence a substituted or unsubstituted alkyl, substituted or unsubstituted cycloalkyl, substituted or unsubstituted alkenyl, substituted or unsubstituted cycloalkenyl, substituted or unsubstituted aryl, substituted or unsubstituted alkoxy, substituted or unsubstituted aryloxy, substituted or unsubstituted heterocylic ring containing one or more heteroatoms, or substituted or unsubstituted fused ring formed between two or more cyclic rings or heteroatom-containing cyclic rings.
25. A method for treating tuberculosis, bacterial infection, or related diseases, which comprises administering a therapeutically effective amount of a compound of any of paragraphs 1-22, or a salt thereof to a subject in need thereof.
26. The method of any of paragraphs 24-25, wherein the subject is a mammal.
27. The method of any of paragraphs 24-26, wherein the subject is a human.
28. The method of any of paragraphs 24-27, wherein the bacterial infection is an infection caused by mycobacterium tuberculosis.
29. The method of any of paragraphs 24-28, wherein the therapeutically effective amount is about 0.001-50 mg per kg body weight daily.
30. The method of any of paragraph 24-29, wherein the therapeutically effective amount is about 1 to about 100 mg once-a-week or twice-a-week.
31. The method of any of paragraphs 24-302, wherein compound of formula (I) is of formula (III) or compound of formula (II) is of formula (IV):

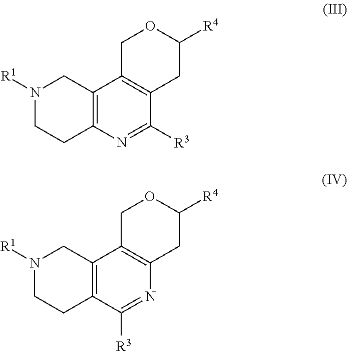

wherein:
R¹ is —(CH₂)ₘR⁶, —C(O)N(H)R⁶, —C(O)R⁶, or —S(O)₂R⁶;
m is 0-6;
R³ and R⁴ are each independently a substituted or unsubstituted alkyl, substituted or unsubstituted aryl, or substituted or unsubstituted heterocylic ring containing one or more heteroatoms; and
R⁶ represents independently for each occurrence a substituted or unsubstituted alkyl, substituted or unsubstituted cycloalkyl, substituted or unsubstituted alkenyl, substituted or unsubstituted cycloalkenyl, substituted or unsubstituted aryl, substituted or unsubstituted alkoxy, substituted or unsubstituted aryloxy, substituted or unsubstituted heterocylic ring containing one or more heteroatoms, or substituted or unsubstituted fused ring formed between two or more cyclic rings or heteroatom-containing cyclic rings.
32. The method of any of paragraphs 24-31, wherein the compound of formula (I) is represented by formula (IIIa), (IIIb), or (IIIc):

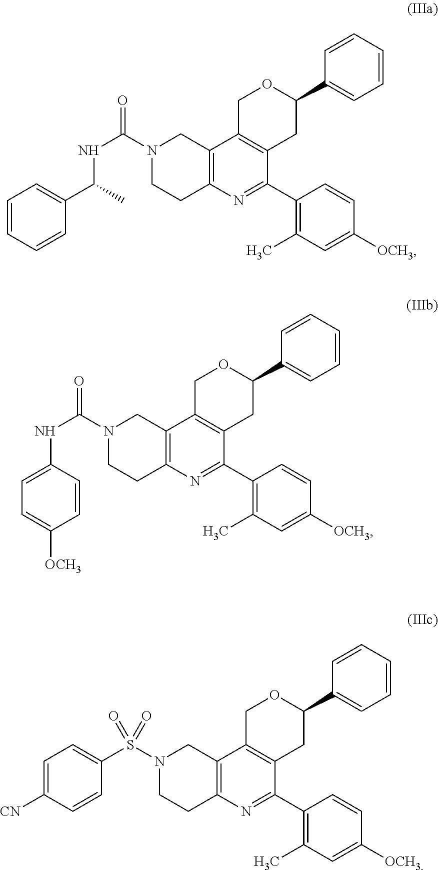

33. The method of any of paragraphs 24-32, wherein the subject is a mammal.
34. The method of any of paragraphs 24-33, wherein the subject is a human.
35. The method of any of paragraphs 24-34, wherein the bacteria infection is an infection by mycobacterium tuberculosis.
36. The method of any of paragraphs 24-35, wherein the therapeutically effective amount is from about 0.001 mg/kg to about 100 mg/kg body weight daily.
37. The method of any of paragraphs 24-36, wherein the therapeutically effective amount is from about 1 mg/kg to about 100 mg/kg body weight at least once-a-week, at least twice-a week, at least thrice-a-week, at least four-times a week, at least five-times a week, or at least six-times a week.
38. The method of any of paragraphs 24-37, wherein said administrating is in the form of an aerosol.
39. The method of any of paragraphs 24-38, wherein the tuberculosis improves as measured by an indication selected from the group consisting of a decrease in fever, a reduction in sputum production, a reduction in wheezing and a reduction in conversion of sputum cultures.
40. The method of any of paragraphs 24-39, wherein the subject in need of such treatment is unresponsive to treatment with one or more antibiotics.
41. The method of any of paragraphs 24-40, wherein the tuberculosis is multiple drug resistant (MDR-TB).
42. The method of any of paragraphs 24-41, wherein said administering results in improvement in pulmonary function tests.
43. The method of any of paragraphs 24-42, further comprising administering a therapeutically effective amount of an antibiotic agent.
44. The method of any of paragraphs 24-43, wherein the antibiotic agent is selected from the group consisting of penicillins, cephalosporins, vancomycins, bacitracins, macrolides, erythromycins, lincosamides, clindomycin, chloramphenicols, tetracyclines, aminoglycosides, gentamicins, amphotericins, cefazolins, clindamycins, mupirocins, sulfonamides, trimethoprim, rifampicins, metronidazoles, quinolones, novobiocins, polymixins, gramicidins, immunocycline, chlortetracycline, oxytetracycline, demeclocycline, methacycline, deoxycycline, minocycline, isoniazid, rifampin, and any salts or variants thereof.

Content of all patents and other publications identified and listed in the specification is explicitly incorporated herein by reference in its entirety for all purposes.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

We claim:

1. A compound of formula (III) or (IV): 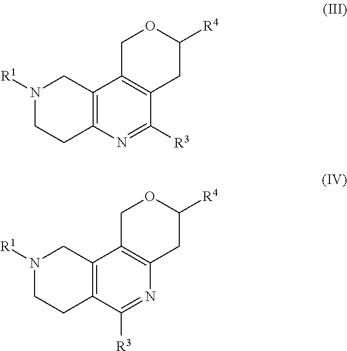
wherein:
R¹ is —(CH₂)ₘR⁶, —C(O)N(H)R⁶, —C(O)R⁶, or —S(O)₂R⁶;
m is 0-6;
R³ and R⁴ are each independently a substituted or unsubstituted alkyl, substituted or unsubstituted aryl, or substituted or unsubstituted heterocylic ring containing one or more heteroatoms; and
R⁶ represents independently for each occurrence a substituted or unsubstituted alkyl, substituted or unsubstituted cycloalkyl, substituted or unsubstituted alkenyl, substituted or unsubstituted cycloalkenyl, substituted or unsubstituted aryl, substituted or unsubstituted alkoxy, substituted or unsubstituted aryloxy, substituted or unsubstituted heterocylic ring containing one or more heteroatoms, or substituted or unsubstituted fused ring formed between two or more cyclic rings or heteroatom-containing cyclic rings.

2. The compound of claim 1, wherein the compound is represented by formula (III).

3. The compound of claim 1, wherein R¹ is —C(O)N(H)R⁶, and R⁶ represents independently for each occurrence a substituted C₁-C₆ alkyl, unsubstituted C₂-C₆ alkenyl, substituted aryl, or unsubstituted 5-member heterocylic ring containing one or more heteroatoms, wherein the heteroatoms are O, N or S.

4. The compound of claim 3, wherein the substituted alkyl is an alkyl group substituted with one or more moieties selected from the group consisting of alkyl, phenyl, indolyl, furanyl, thiofuranyl, pyrrolyl, imidazolyl, and —C(O)OR⁸, wherein R⁸ is hydrogen, or substituted or unsubstituted C₁-C₆ alkyl; and
the substituted aryl is a phenyl group substituted with one or more moieties selected from the group consisting of alkoxy, aryloxy and cyano.

5. The compound of claim 3, wherein R⁶ is in each occurrence independently selected from the group consisting of 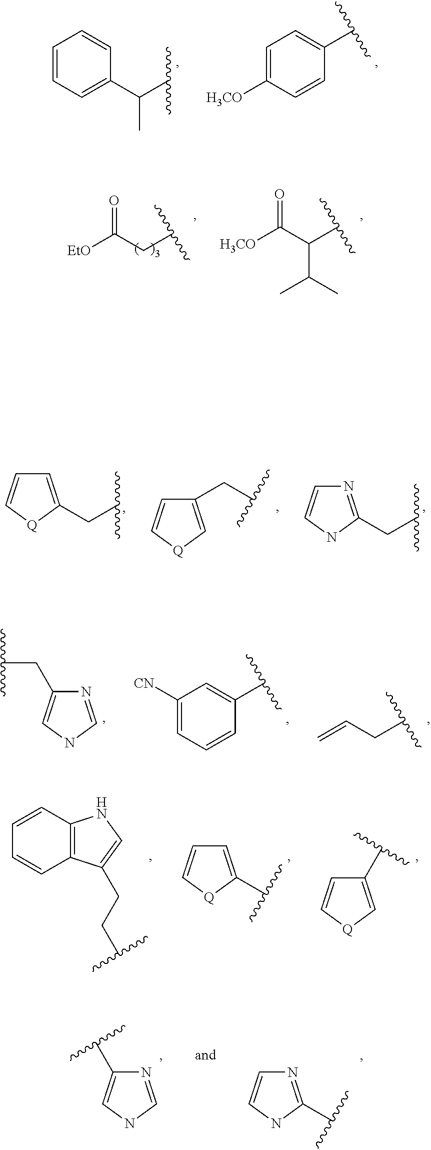
wherein Q is O, S, or N.

6. The compound of claim 1, wherein R³ and R⁴ are each independently a substituted or unsubstituted C₁-C₆ alkyl, or substituted or unsubstituted aryl.

7. The compound of claim 6, wherein R³ is selected from the group consisting of n-butyl, phenyl,

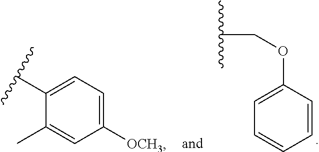

8. The compound of claim 6, wherein R⁴ is selected from the group consisting of methyl, phenyl or

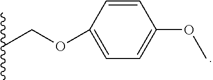

9. The compound of claim 1, represented by formula (IIIa), (IIIb), or (IIIc):

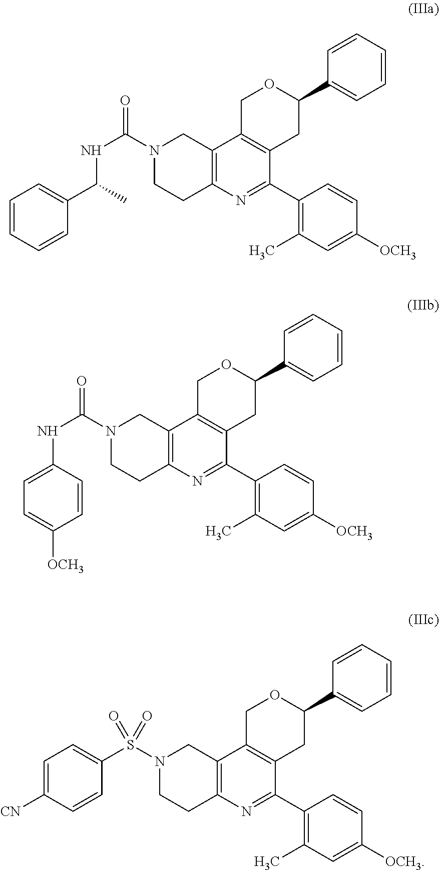

10. A pharmaceutical composition, comprising a therapeutically effective amount of a compound of claim 1 and a pharmaceutically acceptable carrier or excipient.

11. A method for treating tuberculosis, bacterial infection, or related diseases, the method comprising administering a therapeutically effective amount of a compound of formula (I) or (II), or a pharmaceutically acceptable salt thereof to a subject in need of such treatment: 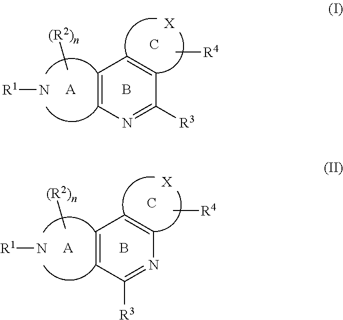
wherein
A and C are each independently a 5-7 member heterocyclic ring, wherein the N and X variables are present at any position in the ring;
R¹ is H, substituted or unsubstituted alkyl, substituted or unsubstituted aryl, —(CH₂)ₘR⁶, —C(O)N(H)R⁶, —C(O)R⁶, or —S(O)₂R⁶;
m is 0-6;
each R² is independently H, alkyl, or aryl;
n is 0-4;
R³ is hydrogen, substituted or unsubstituted alkyl, substituted or unsubstituted aryl, or substituted or unsubstituted heterocylic ring containing one or more heteroatoms;
R⁴ is hydrogen, substituted or unsubstituted alkyl, substituted or unsubstituted aryl, or substituted or unsubstituted heterocylic ring containing one or more heteroatoms, wherein R⁴ is in an ortho-position to X on the heterocyclic ring and with the proviso that R⁴ is H when C is a 5-member ring;
X is O, S, S(O), S(O)₂, or NR⁵, wherein R⁵ is substituted or unsubstituted alkyl or aryl;
and R⁶ represents independently for each occurrence a substituted or unsubstituted alkyl, substituted or unsubstituted cycloalkyl, substituted or unsubstituted alkenyl, substituted or unsubstituted cycloalkenyl, substituted or unsubstituted aryl, substituted or unsubstituted alkoxy, substituted or unsubstituted aryloxy, substituted or unsubstituted heterocylic ring containing one or more heteroatoms, or substituted or unsubstituted fused ring formed between two or more cyclic rings or heteroatom-containing cyclic rings.

12. The method of claim 11, wherein the compound of formula (I) is represented by formula (III):

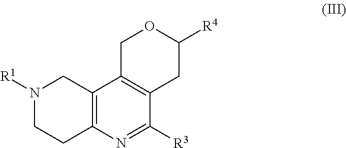

13. The method of claim 12, wherein the compound of formula (III) is represented by formula (IIIa), (IIIb), or (IIIc):

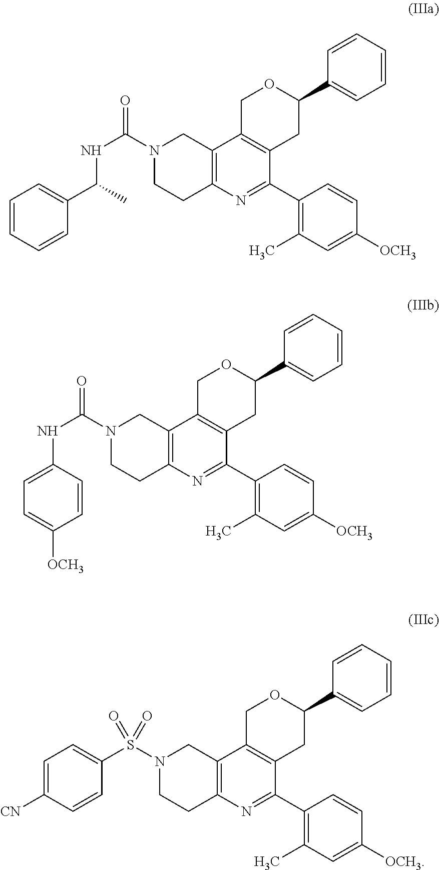

14. The method of claim 11, wherein the bacterial infection is an infection by Mycobacterium tuberculosis.

15. The method of claim 11, wherein the therapeutically effective amount is about 0.001 mg/kg to about 100 mg/kg body weight daily.

16. The method of claim 11, wherein the subject in need of such treatment is unresponsive to treatment with one or more antibiotics.

17. The method of claim 11, further comprising administering a therapeutically effective amount of an antibiotic agent.

18. The method of claim 17, wherein the antibiotic agent is selected from the group consisting of penicillins, cephalosporins, vancomycins, bacitracins, macrolides, erythromycins, lincosamides, clindomycin, chloramphenicols, tetracyclines, aminoglycosides, gentamicins, amphotericins, cefazolins, clindamycins, mupirocins, sulfonamides, trimethoprim, rifampicins, metronidazoles, quinolones, novobiocins, polymixins, gramicidins, immunocycline, chlortetracycline, oxytetracycline, demeclocycline, methacycline, deoxycycline, minocycline, isoniazid, rifampin, and any salts or variants thereof.
